(19) United States
(12) Patent Application Publication
(10) Pub. No.: US 2026/0093034 A1
(43) Pub. Date: Apr. 2, 2026

(54) SYNTHETIC LENSES FOR ULTRASOUND IMAGING SYSTEMS

(71) Applicant: Exo Imaging, Inc., Santa Clara, CA (US)

(72) Inventors: Yusuf HAQUE, Woodside, CA (US); Sandeep Akkaraju, Wellesley, MA (US); Janusz BRYZEK, Santa Clara, CA (US); Andalib CHOWDHURY, San Jose, CA (US); Drake GUENTHER, Hillsborough, CA (US)

(21) Appl. No.: 19/230,035

(22) Filed: Jun. 5, 2025

Related U.S. Application Data
(63) Continuation of application No. 17/341,931, filed on Jun. 8, 2021, now abandoned.
Application No. 17/341,931 is a continuation of application No. PCT/US2020/013530, filed on Jan. 14, 2020.
(60) Provisional application No. 62/792,821, filed on Jan. 15, 2019.

(51) Int. Cl. G01S 15/89; A61B 8/00; B06B 1/06; G01S 7/52

(52) U.S. Cl. CPC G01S 15/8925; A61B 8/4494; A61B 8/54; B06B 1/0622; G01S 7/52079; G01S 15/8927; G01S 15/8993

(57) ABSTRACT

Disclosed herein are ultrasonic transducer systems comprising: an ultrasonic imager comprising a plurality of pMUT transducer elements; and one or more circuitries connected electronically to the plurality of transducer element, the one or more circuitries configured to enable: pulse transmission and reception of reflected signal for the ultrasonic transducer; and control of the ultrasonic transducer, the control of the ultrasonic transducer comprising focusing ultrasonic beam in an elevation direction.

### CROSS-REFERENCE TO RELATED APPLICATIONS

This application is a continuation of U.S. patent application Ser. No. 17/341,931, filed Jun. 8, 2021, which is a continuation of International Application No. PCT/US2020/013530, filed Jan. 14, 2020, which claims the benefit of U.S. Ser. No. 62/792,821, filed Jan. 15, 2019, each of which is hereby incorporated by reference in its entirety.

### BACKGROUND

For ultrasound imaging, transducers are used to transmit an ultrasonic beam towards the target to be imaged and the reflected waveform is received by the transducer and the received waveform is converted to an electrical signal and with further signal processing an ultrasound image is created. Conventionally, for two-dimensional (2D) imaging, the ultrasonic transducer includes a one-dimensional (1D) transceiver array for emitting an ultrasonic beam. A mechanical lens located on top of the array focuses the ultrasound waveform in the elevation plane. Once built, the structural properties, and thus the corresponding functional properties of the array and the mechanical lens, cannot be changed.

### SUMMARY

A piezoelectric sensor has been used for medical imaging for more than two decades. These are typically built using bulk piezoelectric films. These films form piezoelectric elements which are arranged along columns in the azimuth direction. Each column can be driven by transmit drivers. By using different time delays on successive columns, it may be possible to focus transmitted beams in the azimuth direction.

The elevation disposition of the array of piezoelectric elements can permit the beam of the array to be electronically focused into a narrow beam in the elevation plane. The single row of piezoelectric elements of the transceiver array does not enable electronic focusing in the elevation or thickness dimension of the 2D ultrasound image. Traditional 2D ultrasound image is in the azimuth plane with some thickness in the elevation direction (i.e., the conventional technique for restricting the beam to a thin image slice is to mechanically focus the beam in this transverse or elevational dimension, either by contouring the piezoelectric elements in this dimension or lensing each element.) More recently it has been shown that elevational focusing can be achieved by controlling the piezoelectric properties of the elements in this dimension. In this technique, known as shaded polarization, intense, gradated electric fields are uniformly applied to each element to taper the polarization of the piezoelectric elements so that they are most strongly polarized in the center and polarized to a lesser degree toward each end of the element in the elevational direction. The technique may shape the acoustic transmissivity of each piezoelectric element to be greater along the longitudinal center line of the array and lesser toward each elevational side. A significant disadvantage of this technique is the difficulty of precisely controlling the magnitude and gradient of the polarization shading. Other existing techniques where a smaller voltage drive for a part of the array may be used to achieve elevation focus, but with disadvantages. For example, US Patent 2005/0075572 A1 uses a mechanical lens to assist with elevation focus.

Other methods may have the transducer organized in multiple rows. For example, 1.5-dimensional (1.5D), 1.75-dimensional (1.75D) transducers may allow some control on elevation focus using multiple transmissions and receptions and performing receive beam forming using, for example, dual stage beamformers. However, these methods may only allow a limited degree of elevation focus and reduced frame rates of the image due to the need for multiple transmissions and receptions. Further, additional computations may be required, thus increasing power and costs which are not desired in low-cost portable devices that are generally battery powered.

In one aspect, disclosed herein are an ultrasonic imaging systems comprising: a) an ultrasonic transducer comprising a plurality of pMUT transducer elements, each of the plurality of pMUT transducer elements having two or more terminals; and b) one or more circuitries connected to the plurality of pMUT transducer elements, the one or more circuitries electronically configured to enable: i) ultrasonic pulse transmission from the ultrasonic transducer; ii) receiving the reflected ultrasonic signal at the ultrasonic transducer; and iii) electronic control configured to focus the ultrasonic pulse or the reflected ultrasonic signal in an elevation direction. In some embodiments, the plurality of transducer elements comprises an array of transducer elements. In some embodiments, the array is two dimensional. In some embodiments, the array comprises a shape selected from: a rectangular, a square, an annular, an elliptical, a parabolic, a spiral, or an arbitrary shape. In some embodiments, the plurality of transducer elements is arranged in one or more rows and one or more columns. In some embodiments, each transducer element on a column is driven by a multilevel pulse generated by the one or more circuitries. In some embodiments, each transducer element on a column is driven by a sequence of multilevel pulses generated by the one or more circuitries. In some embodiments, pulse magnitude, width, shape, pulse frequency, or their combinations of the multilevel pulse are electrically programmable. In some embodiments, a delay of pulse onset is electrically programmable. In some embodiments, one or more of pulses in the pulse sequence are electrically programmable. In some embodiments, a shape of the multilevel pulse is sinusoidal, digital square, or arbitrary. In some embodiments, a first terminal of the one or more of the plurality of pMUT transducer elements is connected to the one or more circuitries and a second and optionally additional terminal is connected to a bias voltage. In some embodiments, the one or more of the plurality of pMUT transducer elements are poled in two directions on different portions thereof, wherein strength of polarization varies depending on location of the one or more elements of the plurality of pMUT transducer elements on a row, and wherein each of the one or more of the plurality of pMUT transducer elements comprises at least three terminals. In some embodiments, the one or more of the plurality of pMUT transducer elements are poled in only one direction and wherein each of the one or more of the plurality of pMUT transducer elements comprises only two terminals. In some embodiments, poling strength is stronger for center rows and weaker for outer rows, thereby creating apodization in the elevation direction. In some embodiments, the one or more circuitries comprise one or more of: a transmit driver circuit, a receive amplifier circuit, and a control circuit. In some embodiments, the transmit driver circuit is configured to drive the one or more pMUT transducer elements on a column and is driven by signals from a transmit channel, wherein the signals of the transmit channel are delayed electronically relative to delay applied to other transmit channels driving other pMUT transducer elements on different columns. In some embodiments, the one or more pMUT transducer elements on the column operate with a substantially identical delay or different delays. In some embodiments, the control is in real time. In some embodiments, each of the plurality of transducer elements comprises a first lead and a second lead, the first lead electronically connected to the one or more circuitry and the second lead connected to corresponding leads of other transducer elements of the plurality of transducer elements. In some embodiments, the ultrasonic imaging system further comprises an external lens positioned on top of the plurality of transducer elements, the external lens configured to provide additional focus in the elevation direction. In some embodiments, the control circuit is configured to electrically control relative delays between drive pulses for transducer elements located on a same column. In some embodiments, the transmit channel and additional transmit channels are configured to electrically control relative delays between adjacent columns, and wherein the control circuit is configured to set relative delays for a first number of transducer elements on the column such that the first number of transducer elements in a same row share a substantially similar relative delay to a second number of transducer elements of a starting row. In some embodiments, the transmit channel and additional transmit channels are configured to electronically control relative delays between adjacent columns and wherein the control circuit is configured to set relative delays for transducer elements on the column such that a first number of transducer elements in a same row have independent delays compared to a second number of transducer elements on the same row for other columns. In some embodiments, the control circuit is configured to electrically control relative delays of a column to be symmetrical with respect to a transducer element at a center row of the column. In some embodiments, the control circuit is configured to electrically control relative delays to be linearly increasing in a column thereby steering the ultrasonic beam in the elevation direction. In some embodiments, the control circuit is configured to electrically control relative delays thereby controlling slice thickness in the elevation direction. In some embodiments, the plurality of transducer elements comprises a top section, a central section, and a bottom section, each of which comprise a number of rows and a number of columns for the pulse transmission and reception of the reflected ultrasonic signal, wherein the pulse transmission and reception of the reflected ultrasonic signal from the sections are used for focusing the reflected ultrasonic signal in an azimuth direction using a first beamformer, and wherein elevation focus is achieved using a second beamformer. In some embodiments, scan lines from the sections are synchronized to minimize movement errors in target being imaged by completing scanning of an entire column before proceeding with scans of succeeding columns. In some embodiments, a focal distance in the elevation direction is electronically programmed. In some embodiments, the pulse transmission and reception of the reflected signal of the top section and the bottom section are performed simultaneously. In some embodiments, the movement errors in the target being imaged are minimized by performing parallel beamforming to develop the scan lines. In some embodiments, the elevation focus and elevation apodization is performed electronically to minimize movement errors. In some embodiments, the multilevel pulse is used to implement apodization electronically by using lower amplitude drives for outer rows and higher amplitude drives for central rows. In some embodiments, the top section, the central section, or the bottom section comprises more than one subsections, each of which comprise a number of rows and columns for pulse transmission and reception of reflected signal. In some embodiments, the plurality of transducer elements comprises 5 sections, wherein two outer sections transmitting and receiving azimuthally focused beams is followed by two inner sections transmitting and receiving the azimuthically focused beams and the central section transmitting and receiving the azimuthically focused beams, forming scan lines using a first level beamformer, and achieving elevation focus using a second level beamformer. In some embodiments, apodization is implemented in the elevation direction electronically. In some embodiments, the ultrasonic transducer exhibits a bandwidth that is not materially limited by signal losses caused by losses in a mechanical lens. In some embodiments, two of the plurality of pMUT transducer elements are addressed together, the two elements being adjacent on a same row of the one or more rows and wherein the plurality of transducer elements comprises a top section, a central section, and a bottom section, each of which comprise a first number of rows and a second number of columns for the ultrasonic pulse transmission and reception of the reflected ultrasonic signal, wherein the ultrasonic pulse transmission and reception of the reflected ultrasonic signal from the sections are used for focusing the reflected ultrasonic signal in an azimuth direction using a first beamformer, and wherein elevation focus is achieved using a second beamformer, and wherein, for imaging using a B mode, a receive channel is assigned to two transducer elements combined effectively on a same row, where the 2 elements now act as 1 effective element, and a portion of the rows from the top and bottom containing this combined elements are connected together, and another channel is assigned to two transducer elements of the central section, consisting of a few rows. In some embodiments, 2N receive channels are used to address N columns. In some embodiments, all of the plurality of transducer elements are operated on to generate pressure with elevation focus in a transmit operation, and wherein in a receive operation, all of the plurality of transducer elements are used to reconstruct an image with focusing in the azimuth direction and an elevation plane. In some embodiments, transmit apodization is used in the elevation plane. In some embodiments, the elevation focus is dynamic and is steered in the elevation plane. In some embodiments, no mechanical lens is used. In some embodiments, one or more of the pMUT transducer elements comprise multiple subelements configurable for simultaneous transmit and receive operations. In some embodiments, one or more of the pMUT transducer elements comprise multiple subelements and wherein the multiple subelements have different resonant frequency responses. In some embodiments, each of the plurality of pMUT transducer elements has at least two terminals. In some embodiments, the control circuit is configured for determining relative delays for transducer elements on a column, and wherein the control circuit comprises a coarse delay circuit configured to set a coarse delay and a fine delay circuit configured to set a fine delay. In some embodiments, beam steering is achieved using the coarse delay circuit and elevation focus is achieved using the fine delay circuit. In some embodiments, the fine delay for a column is independent of fine delays on other columns. In some embodiments, the control circuit is configured to electrically control relative delays to be piecewise linearly increasing or decreasing in a column, and wherein a number of piecewise linear delay segments is an integer that is no less than 2. In some embodiments, the control circuit is implemented on an ASIC. In some embodiments, the control circuit is configured to electrically control relative delays along a column to be a summation of a linear delay and an arbitrary, fine delay. In some embodiments, the linear delay and arbitrary fine delays of the column are independent from other linear delay and arbitrary fine delays of other columns of the ultrasonic transducer, thereby allowing for arbitrary steering and focusing in three dimensions. In some embodiments, each of the plurality of pMUT transducer element exhibits a plurality of modes of vibration, wherein one or only one mode of vibration is triggered when an input stimulus is bandlimited to be less than frequencies of others of the plurality of modes of vibration adjacent to said one or only mode of vibration. In some embodiments, each of the plurality of pMUT transducer element exhibits a plurality of modes of vibration, where frequencies generated from a first of the plurality of modes of vibration overlaps with the frequencies from the second plurality of modes of vibration. In some embodiments, each of the plurality of pMUT transducer element exhibits a plurality of modes of vibration simultaneously when driven by a wide band frequency input that includes center frequencies of the plurality of modes of vibrations. In some embodiments, the one or more circuitries is electronically configured to enable electronic control of apodization in the elevation direction. In some embodiments, each of the plurality of pMUT transducer element is fabricated on a same semiconductor wafer substrate and connected to sensing, drive and control circuitry in close proximity thereto.

In some embodiments, one or more circuitries are electronically configured to develop B mode imaging in the azimuth plane in one operation, wherein delays from a transmit beamformer are applied in an azimuth direction to selected elements, and further configured to develop B mode imaging in an orthogonal plane and further configured to develop B mode imaging in an orthogonal plane, by using the transmit beamformer to adjust delays in the elevation direction in a subsequent operation to display biplane images formed on 2 orthogonal axes using a synthetic aperture combination technique. In some embodiments, when imaging in the azimuth plane, elevation focus is achieved by adding additional delays on elements on a column and when forming images on the elevation plane, adding additional delays on the azimuth axis on elements on rows to enable additional focus in the azimuth plane.

In another aspect, disclosed herein are methods of performing 3D imaging using the ultrasonic imaging system herein, that comprises a) transmitting an ultrasonic pulse by the plurality of pMUT transducer elements, comprising: applying a first plurality of delays in an azimuth direction for a set of transmissions with a particular steering angle in the elevation direction controlled by a second plurality of delays applied to more than one of the plurality of pMUT transducer elements on a same column; and repeating a) for a predetermined number of times with an additional steering angle in the elevation direction for each repetition of a); receiving a reflected ultrasonic signal by the plurality of pMUT transducer elements; and reconstructing an image using the received reflected ultrasonic signal from the plurality of pMUT transducer elements. In some embodiments, the delays within the first plurality of delays are equal in magnitude and the delays within the second plurality of delays are equal in magnitude. In some embodiments, applying a first plurality of delays further comprises: a) focusing on an azimuth plane by varying a magnitude of one or more delays within the first plurality of delays along the azimuth; and focusing or steering a beam in the elevation direction by varying a magnitude of one or more delays within the second plurality of delays for more than one of the plurality of pMUT transducer elements along a particular column. In some embodiments, the set of transmissions has a particular focus. In some embodiments, the image is three-dimensional and represents a volume. In some embodiments, the delays within the first plurality of delays are not all equal in magnitude and the delays within the second plurality of delays are not all equal in magnitude. In some embodiments, the predetermined number is fewer than 100. In some embodiments, the predetermined number is greater than 1000.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

A better understanding of the features and advantages of the present subject matter will be obtained by reference to the following detailed description that sets forth illustrative embodiments and the accompanying drawings.

FIG. 1 shows an exemplary schematic diagram of an ultrasonic system herein including a transducer with a pMUT array used to transmit and receive ultrasonic beams, electronics to control the pMUT array, other computational, control, and communication electronics, a display unit and a recording unit, with the pMUT array directed at a target to be imaged.

FIG. 2 shows an exemplary schematic diagram of an ultrasonic transducer here.

FIG. 3A shows an exemplary schematic diagram of a piezoelectric micro machined transducer (pMUT) element with 2 conductors.

FIG. 3B shows an exemplary schematic diagram of a pMUT element comprising two sub elements, each sub elements with 2 or more electrodes.

FIG. 3C shows an exemplary schematic of a pMUT element with 2 sub elements, each sub element with 2 electrodes, wherein the first electrode of first sub element is connected to one of the electrodes of the second element and the second electrode of the first element connected to the remaining electrode of the second sub element.

FIG. 4 shows an exemplary diagram of the pMUT array of an ultrasonic transducer system herein.

FIG. 5A shows an exemplary cross-section of a piezoelectric element of the pMUT array herein.

FIG. 5B shows an exemplary symbolic representation of the piezoelectric element of FIG. 5A.

FIG. 6 shows dipole orientation in piezoelectric element herein on unpoled state and during poling and after poling.

FIG. 7 shows exemplary connection of a piezoelectric element herein to a low noise amplifier (LNA) during receive mode with symbolic connection arrangement.

FIG. 8A shows an exemplary embodiment of a 2D array of pMUTs with one common ground or biasing electrode for electrically adjustable line transducers in which lines can be in vertical or horizontal direction and size of lines (e.g., number of pMUT element in a line) can be electrically programmable.

FIG. 8B shows an exemplary embodiment of a 2D array of pMUTs with connections shown to bias voltage and/or actively driven terminals.

FIG. 9 shows an exemplary embodiment of a line transducer that shows multiple ground and biasing electrodes to enable differential poling directions per pMUT element.

FIG. 10A shows an exemplary pMUT array with multiple membranes in each piezoelectric element with capability of using different poling directions for piezo material controlling membranes and different poling strength per row.

FIG. 10B shows an exemplary implementation of FIG. 10A, wherein biasing connections after poling operation is shown.

FIG. 11A shows an exemplary schematic diagram of interconnection of 2 pMUT elements to an ASIC containing transmit and receive drivers and other functions.

FIG. 11B shows an exemplary schematic diagram of the ASIC of FIG. 11A in which one column of electronics interfaces directly to one column of pMUTs, to constitute a composite larger transducer element.

FIGS. 12A and 12B show exemplary schematic diagrams of ultrasonic transducers that focus in the elevation direction disclosed herein.

FIG. 13A shows an exemplary schematic diagram of an ultrasonic transducer with transducer elements that are organized on M rows and N columns, the transducer comprises of three strips that comprises of rows and/or columns, each of the strips can be selected to be driven separately and where columns in each strip share the same drive by transmit driver(s).

FIG. 13B shows an exemplary schematic of an ultrasonic transducer with transducer elements that are organized in rows and columns; two elements on a row are effectively combined together for transmit and receive purposes, and the transducer comprises three portions of transducer elements in rows and/or columns, the top and bottom portions of the transducer can be driven by one channel for transmit and/or receive operations, while the center portion can be driven by a different channel for transmit and/or receive operations.

FIG. 14 shows an exemplary schematic diagram of a number of scan lines that make up an ultrasonic image frame.

FIG. 15 shows an exemplary schematic diagram of obtaining a scan line of FIG. 14.

FIG. 16 shows an exemplary schematic diagram of obtaining elevation focus using delays applied to different strips.

FIG. 17A shows an exemplary schematic diagram of a delay circuit herein with multiple flip flops, providing fine delay(s) to elements on a column

FIG. 17B shows an exemplary schematic diagram of a delay circuit herein, providing coarse delay(s) to elements on a column.

FIG. 17C shows an exemplary schematic diagram of a delay circuit that provides coarse and/or fine delay(s) to elements on a column.

FIG. 17D shows details of additional circuitry of FIG. 17C.

FIG. 18A shows a diagram with beam steering or beam focusing in the azimuth direction using delay in the azimuth direction from transmit channels.

FIG. 18B shows an exemplary schematic diagram of transducer elements and their delays, the delays can be programmed electronically and can be substantially similar for more than one column of the transducer elements.

FIG. 19 shows an exemplary schematic diagram of transmit drive pulses with delays for a column of transducer elements with delay symmetry around a central element

FIG. 20 shows an exemplary schematic diagram of transmit drive pulses with delays for transducer elements of different columns.

FIG. 21 shows an exemplary schematic diagram of generating different delays using internal counter signals.

FIG. 22 shows an exemplary schematic diagram of a pulsar with two digital inputs that generates an output as transmit drive pulse(s).

FIG. 23A shows exemplary elevation beamplots of a simulated 24×128 matrix array of transducer elements with 0° lateral steering (left panel) and 45° lateral steering (right panel), which indicate the differences of multiple methods of providing focusing in the elevation direction in comparison to no focusing in the elevation direction.

FIG. 23B shows an exemplary sparse transmit scheme allowing for transmit elevation focusing with a 24×128 2D array of transducer elements, in which the shaded circles can be the active transducer elements per column and elevation symmetry can be used (assuming focusing along the elevation plane of symmetry). This transmit scheme can output approximately ⅓ less pressure than when using all 24×128 active elements.

FIG. 24A shows a schematic diagram of an imaging assembly according to embodiments of the present disclosure.

FIG. 24B shows an exemplary embodiment of the transducer disposed on a substrate and ASIC on another substrate and means of interconnection.

FIG. 25 illustrates a schematic diagram of an array of piezoelectric elements capable of performing two- and three-dimensional imaging according to embodiments of the present disclosure.

FIG. 26 illustrates a schematic diagram of an array of piezoelectric elements according to embodiments of the present disclosure.

FIG. 27 illustrates a schematic diagram of an array of piezoelectric elements according to embodiments of the present disclosure.

FIG. 28 illustrates a schematic diagram of an array of piezoelectric elements according to embodiments of the present disclosure.

FIG. 29 illustrates a schematic diagram of an array of piezoelectric elements according to embodiments of the present disclosure.

FIG. 30 illustrates a schematic diagram of an array of piezoelectric elements according to embodiments of the present disclosure.

FIG. 31 illustrates a schematic diagram of an array of piezoelectric elements according to embodiments of the present disclosure.

FIG. 32 illustrates a schematic diagram of an array of piezoelectric elements according to embodiments of the present disclosure.

FIG. 33A illustrates a schematic diagram of an imaging system according to embodiments of the present disclosure with hardwired connections for piezoelectric elements on a column.

FIG. 33B illustrates a schematic diagram of an imaging system according to embodiments of the present disclosure with programmable transmit and receive capability for piezoelectric elements on a column.

FIG. 34A illustrates a schematic diagram of an imaging system according to embodiments of the present disclosure with hardwired piezoelectric elements on a column.

FIG. 34B illustrates a schematic diagram of an imaging system according to embodiments of the present disclosure with programmable transmit and receive capability of piezoelectric elements on a column.

FIG. 35A shows an embodiment of a piezoelectric element coupled to a circuit element according to embodiments of the present disclosure.

FIG. 35B shows an exemplary embodiment of a piezoelectric element coupled to a circuit element according to embodiments of the present disclosure, wherein the piezoelectric element has programmable transmit and receive capability.

FIG. 36 shows a circuit for controlling multiple piezoelectric elements according to embodiments of the present disclosure.

FIG. 37 shows a circuit for controlling multiple piezoelectric elements according to embodiments of the present disclosure.

FIG. 38 shows a transmit drive signal waveform according to embodiments of the present disclosure.

FIG. 39A shows a transmit drive signal waveform according to embodiments of the present disclosure.

FIG. 39B shows a transmit drive signal waveform according to embodiments of the present disclosure, wherein TxB CLK is a high-speed clock that can be used to generate TxA and TxB waveform which are generated for pulse output for transmit channels.

FIG. 40 shows a transmit drive signal waveform according to embodiments of the present disclosure.

FIG. 41 shows input/output signals of various circuits in an imaging assembly according to embodiments of the present disclosure.

FIG. 42A shows a plot of the amplitude of a transmit pressure wave as a function of angle according to embodiments of the present disclosure.

FIG. 42B shows windows for apodization process according to embodiments of the present disclosure.

FIG. 43 shows a schematic diagram of an imaging assembly according to embodiments of the present disclosure.

FIG. 44 shows particular steering angles of the transducers according to embodiments of the present disclosure.

### DETAILED DESCRIPTION

Traditionally, a 2D ultrasound image can be created by employing a variety of algorithms, such as those described by Fredrik Lingvall. Lingvall, F., 2004. Time-domain Reconstruction Methods for Ultrasonic Array Imaging: A Statistical Approach [see http://www.signal.uu.se/Publications/pdf/fredrik_thesis.pdf]. One example of this is using relative delay for driving signals along the columns of piezoelectric elements in the azimuth direction. Beams can be focused in the azimuth direction electronically by altering electronically programmable delay applied to a signal for different columns in the azimuth direction. However, focus in a direction orthogonal to the azimuth direction (e.g., the elevation direction) typically is achieved by using a mechanical lens. A mechanical lens may allow only one focus at a time, thus different elevation focuses may require different designs of the lens. Further, a fixed mechanical lens does not provide the focus required for 3D ultrasound imaging.

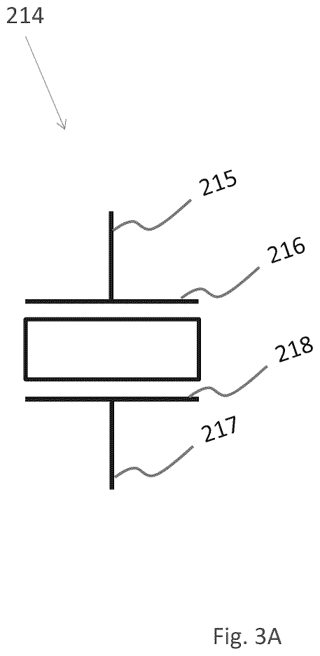

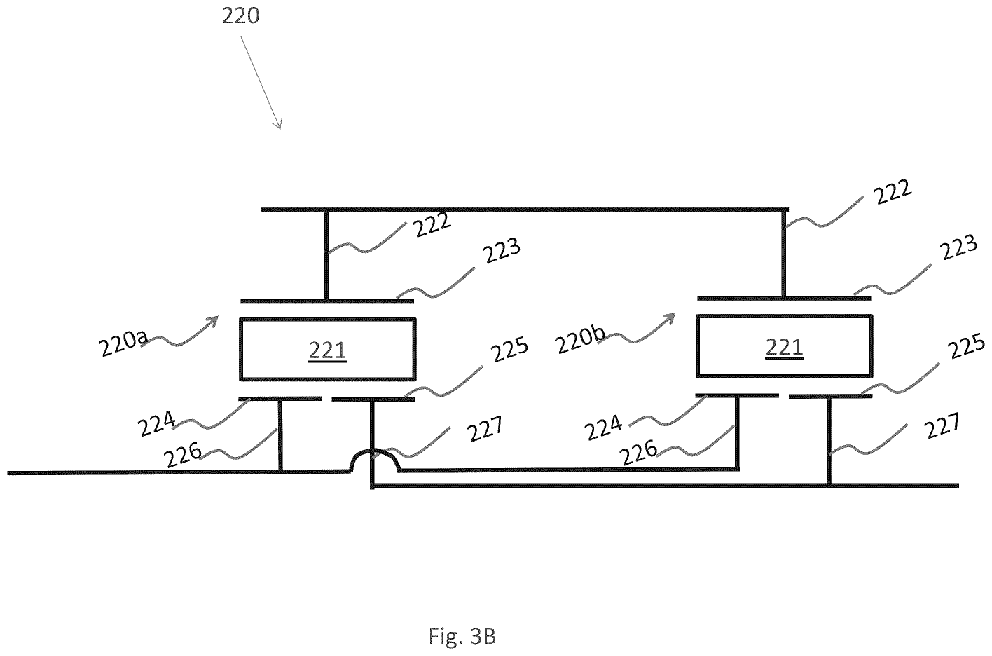

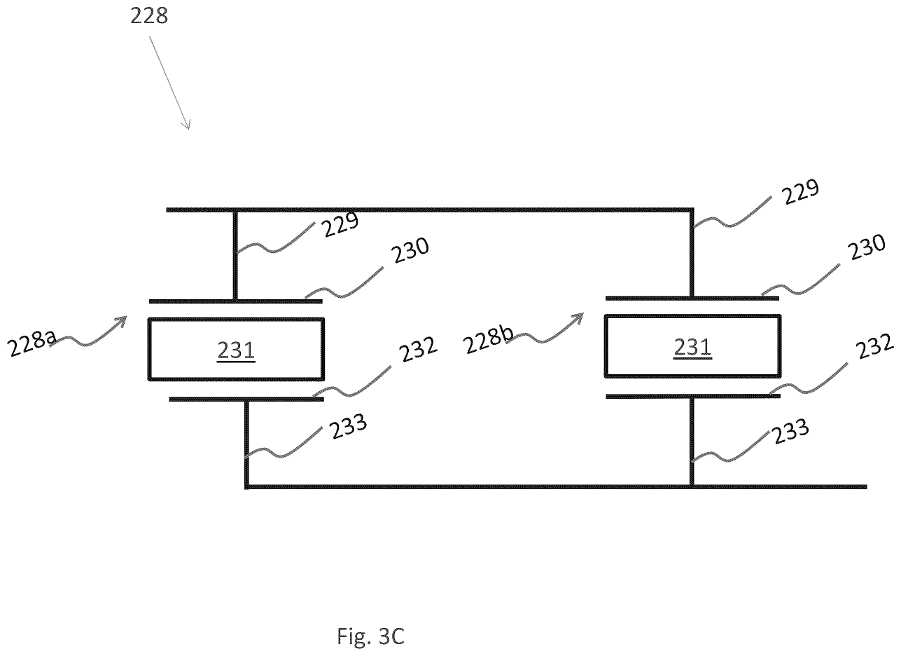

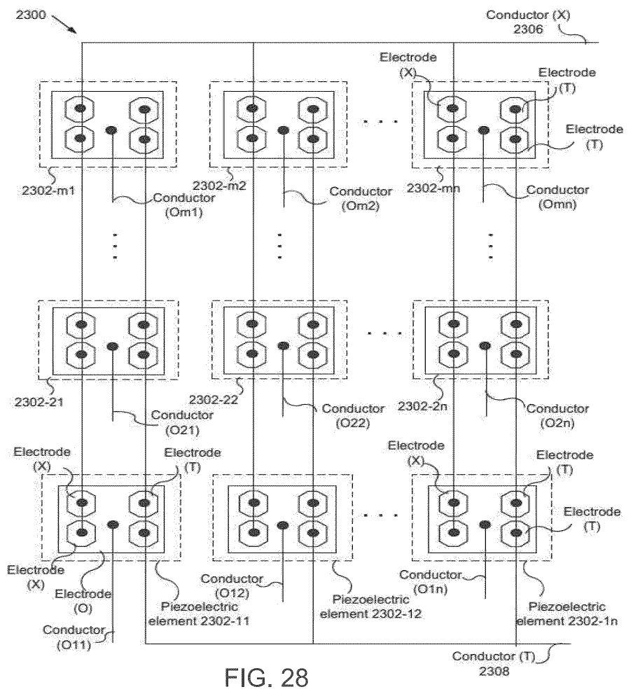

3D ultrasonic imaging has been too complicated, expensive, and power-hungry for implementation in existing portable ultrasonic imaging systems. Disclosed herein in some embodiments are systems and methods configured for enabling low cost, low power, portable high resolution ultrasonic transducers and ultrasound imaging systems configured for both 2D and 3D ultrasonic imaging. Enabling these low-cost high-performance systems can be dependent on using pMUTs that can be manufactured on a semiconductor wafer in high volume and low cost similar to high volume semiconductor processes. In exemplary embodiments, such pMUTs are arranged in a 2D array where each element in the array is connected to an electronic circuit, where the pMUT array and the circuit array are aligned together on different wafers and integrated together to form a tile, where each piezo element is connected to a controlling circuit element, where each piezo element may have 2 or more terminals as shown in FIGS. 3B and 3C. These pMUTs also may exhibit high bandwidth, making these transducers suitable for wideband imaging unlike prior art piezo bulk transducers. Legacy transducers may have limited bandwidth requiring different transducers to be used for different frequency ranges. Therefore, having one transducer covering a wide range of frequencies, such as 1 MHz to 12 MHz or larger can provide for greater user convenience when examining patients, where a user may not have to switch to a different transducer when examining different organs that require widely different frequencies. This can result in cost savings. Wide band behavior can be achieved in pMUTs in the present disclosure in at least 2 different ways. In some embodiments, a transducer element may comprise 2 or more subelements, wherein each subelement resonates at a different center frequency. Together as a composite, the composite element can cover a larger band (see FIG. 28 as an example). In other embodiments, a membrane can be designed such that the membrane can support multiple modes of resonance in one membrane. The resonance can have a primary mode where resonance occurs at a certain frequency. Other resonances may also exist on the membrane, for example, a second and a third resonance. These resonances may or may not be harmonically related. The bandwidth around these resonances can overlap with bandwidth around other resonances, thereby enabling an overall wide bandwidth. If the input signal to the pMUT is bandlimited, e.g., to one resonance, then the other resonances may not occur. The transducer element can exhibit multiple modes of vibration simultaneously when driven by a wide band frequency input that includes center frequencies of the various resonances.

Additionally, existing transducers utilizing a mechanical lens for elevation focusing can also suffer attenuation losses in the lens, thereby reducing image quality. With the exemplary synthetic lenses herein, no mechanical lens is required. Sometimes, a slightly curved deep focus weak lens may be used or instead, a flat thin impedance matching layer can be used on top of the transducer. This may vastly improve attenuation losses.

Instead of using fixed mechanical lenses, the imaging systems disclosed herein use electronic lenses that advantageously eliminate the need to build a mechanical lens with a fixed focal length. Further, the electronic lenses disclosed herein allow great flexibility of being able to alter the focal length in the elevation plane and allow dynamic focus as a function of depth. Further, with apodization, side lobes in the elevation direction can be suppressed, allowing better control of the elevation slice thickness. Electronic real-time control of apodization in the elevation control may advantageously allow side lobe suppression in the elevation direction electronically.

Disclosed herein, in some embodiments, are ultrasonic imaging systems configurable to focus in the elevation direction. Disclosed herein, in some embodiments, are ultrasonic imaging systems configurable to allow electronic elevation control with programmable delay along columns and/or rows. In some embodiments, the electronic control occurs when programmable delays are inserted in the transmit drive circuit driving individual elements on columns.

Disclosed herein, in some embodiments, transducer elements, (e.g., pMUT elements), thus piezoelectric elements of the transducer element, are organized into multiple rows (each row is along the azimuth direction) and columns (each column is along the elevation direction) in two dimensions. In some embodiments, a section including one or multiple rows around a central section of rows can be focused along the azimuth direction. In one single transmission and reception, the data generated from this section can be focused in the azimuth direction, generating intermediate data. In an additional transmission and reception, data from multiple sections can be focused in the elevation direction. This process may improve slice thickness in the elevation direction. In some embodiments, such process can be aided by applying apodization of ultrasonic pulse(s).

### Certain Definitions

Unless otherwise defined, all technical terms used herein have the same meaning as commonly understood by one of ordinary skill in the art to which the described subject matter belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by about 10%, 5%, or 1%, including increments therein.

In some embodiments, the imagers (interchangeably here as “transducers”) herein can be used to perform but is not limited to perform: 1D imaging, also known as A-Scan, 2D imaging, also known as B scan, 1.5D imaging, 1.75D imaging, 3D and Doppler imaging. Also, imager herein can be switched to various imaging modes which are pre-programmed. Also, biplane imaging mode may be implemented using transducers herein.

In some embodiments, transducer elements herein (e.g., pMUT elements) are interchangeable with transceiver element, piezoelectric element, and piezo element. In some embodiments, the transducer element herein includes one or more of: a substrate, a membrane suspending from the substrate; a bottom electrode disposed on the membrane; a piezoelectric layer disposed on the bottom electrode; and one or more top electrodes disposed on the piezoelectric layer.

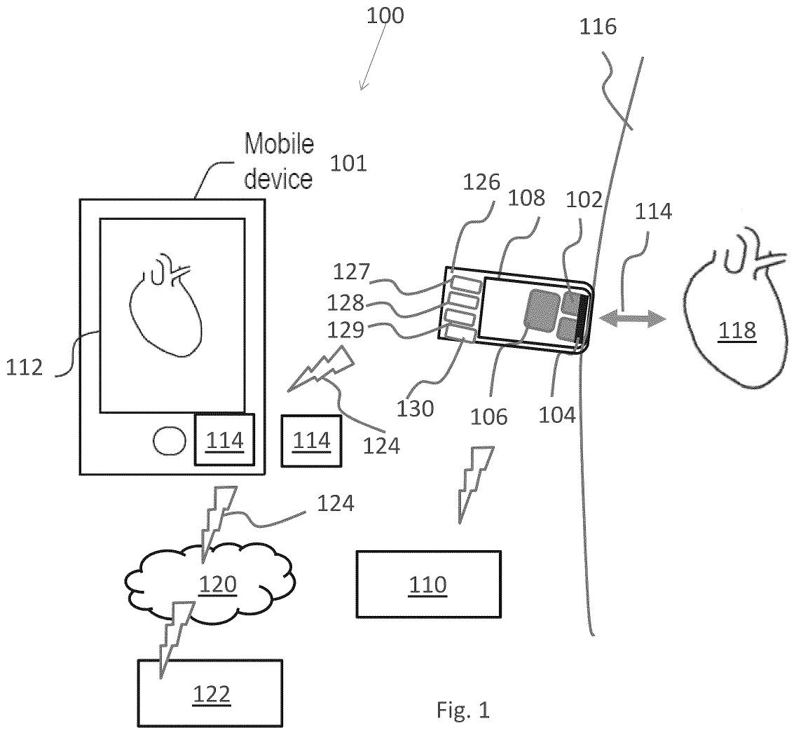

FIG. 1 shows an exemplary embodiment of the ultrasonic imaging system 100 disclosed herein. In this embodiment, the image system includes a portable device 101, the device 101 having a display unit 112, a data recording unit 114 with connection enabled by communication interface to a network 120 and external databases 122, such as electronic health records. Such connection to external data sources may facilitate medical billing, data exchange, inquiries or other medical related information communication. In this embodiment, the system 100 includes an ultrasonic imager (interchangeable as “probe” herein) probe 126 which includes ultrasonic imager assembly (interchangeably as “tile assembly” herein) 108, where the ultrasonic tile has arrays of pMUTs 102 fabricated on a substrate. The array(s) of pMUTs 102 are configured to emit and receive ultrasonic waveforms under an electronic control unit, e.g., an application specific integrated circuit (ASIC) 106 located on the imager and another control unit 110.

In this particular embodiment, the display unit 112 and/or at least part of the electronic communication control unit 110 may be located on the assembly 108. In some embodiments, the display or part of the control unit 110 could be external to the imager but connected to the ultrasonic imager assembly 108 and its elements therewithin with a wired communication interface and/or a wireless communication interface 124. In some embodiments, the display 112 may have an input device, e.g., touch screen, a user-friendly interface, e.g., graphical user interface (GUI), to simplify user interaction.

In the same embodiment, the pMUT array 102 is coupled to an application specific integrated circuit (ASIC) 106 located on another substrate and in close proximity to the pMUT array 102. The array may also be coupled to different impedance material and/or impedance matching material 104 which can be placed on top of the pMUT array. In some embodiments, the imager 126 includes a rechargeable power source 127 and/or a connection interface 128 to an external power source, e.g., using a USB Power Delivery interface that is compatible with signaling protocols in other USB standards such as USB2 or USB 3. In some embodiments, the recharging method is wireless. In some embodiments, the imager 126 includes an input interface 129 for an ECG signal for synchronizing scans to ECG pulses. In some embodiments, the imager 126 has an inertial sensor 130 to assist with user guidance.

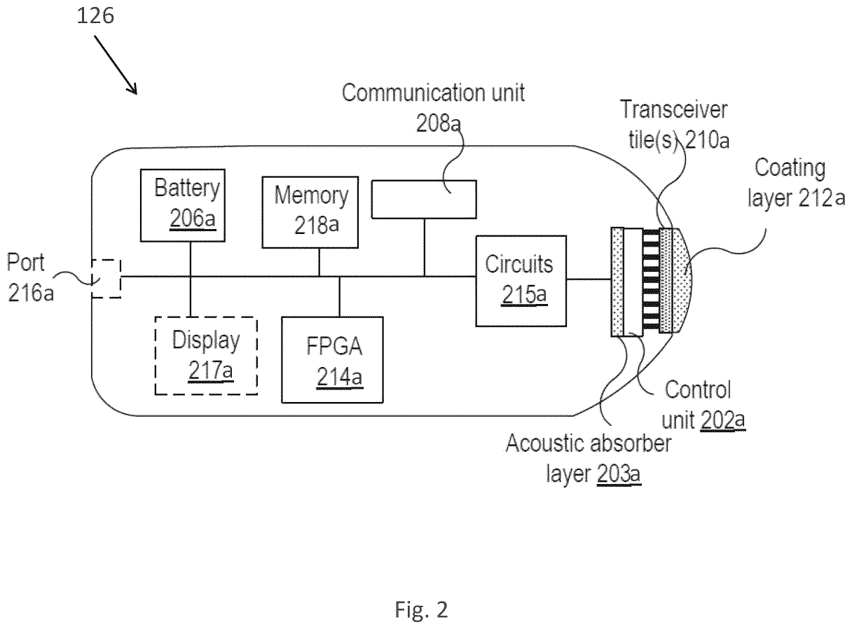

The arrow 114 shows ultrasonic transmit beams from the imager assembly 108 targeting a body part 116 and an imaging a volume element 118. The transmit beams are reflected by the target being imaged and enters the imager assembly 108 as indicated by arrow 114. In addition to ASIC 106, the imaging system 100 may include other electronic control, communication and computational circuitry 110. It is understood that the ultrasonic imager 108 can be one self-contained unit as shown in FIG. 1, or it may include physically separate, but electrically or wirelessly connected elements, such as part of the electronic control unit 110. An example of this is shown in FIG. 2.

FIG. 2 shows a schematic diagram of the imager 126 according to embodiments of the present disclosure. As depicted in FIG. 2, the imager 126 may include: a transceiver tile(s) 210a for transmitting and receiving pressure waves; a coating layer 212a that operates as a lens for steering the propagation direction of and/or focusing the pressure waves and also functions as an impedance interface between the transceiver array and the human body; the lens 212 may also cause attenuation the signal exiting the transducer and also entering the transducer and therefore it is also desirable to keep this to a minimum; when elevation control is electronic, this lens may not be needed and can be replaced by a thin protective impedance matching layer only, where losses are only minimal; a control unit 202a, such as ASIC chip (or, shortly ASIC), for controlling the transceiver tile(s) 210a and coupled to the transceiver tile(s) 210a by bumps. The combination of the transceiver array with the ASIC connected to it is called a tile. Field Programmable Gate Arrays (FPGAs) 214a for controlling the components of the imager 126, a circuit(s) 215a, such as Analog Front End (AFE), for processing/conditioning signals; an acoustic absorber layer 203 for absorbing waves that are generated by the transceiver tile(s) 210a and propagate toward the circuit 215a; in certain embodiments, the acoustic absorber layer is located in between the transducer and the ASIC; in certain embodiments, these acoustic absorber layers are not needed; a communication unit 208a for communicating data with an external device, such as the device 101, through one or more ports 216a; a memory 218a for storing data; a battery 206a for providing electrical power to the components of the imager; and optionally a display 217a for displaying images of the target organs.

During operation, the user may cause the pMUTs 102 surface, covered by interface material 104 to make contact with a body part area upon which ultrasonic waves are transmitted towards the target 118 being imaged. The imager receives reflected ultrasonic beams from the imaging target and processes them or transmits them to an external processor for image processing and/or reconstruction, and then to a portable device 101 for displaying an image. Other data may also be collected, calculated, derived, and displayed on the display to a user.

FIG. 1 shows an exemplary embodiment of the portable ultrasonic imaging system 100 herein including an image probe (interchangeably herein as the transducer) 126. The probe may contain pMUT imager assembly 108 connected to electronic units e.g., control unit 202a in FIG. 2. The probe 102 communicates with external display unit 204 using communications interface and means 124.

This communications means can be a cable or wireless connections. For wired connections, many protocols for data interchange such as USB2, Lightning and others can be used. Similarly, for wireless communications, commonly used protocol such 802.11 or other protocols can be used. Similarly, the data recording unit 114 can also be external to the probe and can also communicate with probe 126 using wireless or wired communication means.

When using the imager, for example to image human or animal body parts, the transmitted ultrasonic waveform is directed towards the target. Contact with the body is achieved by holding the imager in close proximity of the body, usually after a gel is applied on the body and the imager placed on the gel, to allow superior interface of ultrasonic waves being emitted to enter the body and also for ultrasonic waveforms reflected from the target to reenter the imager, where the reflected signal is used to create an image of the body part and results displayed on a screen, including graphs, plots, statistics shown with or without the images of the body part in a variety of formats.

It should be noted that the probe 126 may be developed with certain parts of being physically separate and connected through a cable or wirelessly. As an example, in this particular embodiment, the pMUT assembly and the ASIC and some control and communications related electronics could reside in a unit often called a probe. The part of the device or probe that makes contact with the body part contains the pMUT assembly.

FIG. 3A shows a cross section of a schematic diagram of a conventional piezoelectric element 214. In this embodiment, the piezoelectric element has 2 electrodes, the first electrode 216 is connected to a signal conductor 215 and the second electrode 218 is connected to a second conductor 217 and can be commonly connected to ground or other DC potential.

Piezoelectric elements have been used for decades for ultrasonic medical imaging. However, the piezoelectric element can be thick, for example, approaching around 100 μm and typically may require +100V to −100V alternative current (AC) drive across it to create an ultrasonic pressure wave of sufficient strength to enable medical imaging. The frequency of this AC drive signal may be around the resonating frequency of the piezoelectric structures, and can be above 1 MHz for medical imaging applications.

In some embodiments, the power dissipated in driving the piezoelectric element is proportional to C*V², where C is capacitance of the piezo element and V is the maximum voltage across the piezoelectric layer. When transmitting, a multiplicity of piezoelectric elements can be driven together with somewhat different delays to focus a beam or to steer a beam. The simultaneous drive of many elements can cause temperature to rise on the surface of the elements. It is highly desirable or required not to exceed a threshold temperature so as not to injure the subject being imaged. Thus, this threshold temperature limits the number of elements that can be driven and the time period for which they can be driven.

Disclosed herein, in some embodiments, the piezoelectric elements are much thinner, approximately typically 5 μm or less thick, compared to 100 μm thickness of conventional bulk piezo elements. Such large decrease in thickness may enable use of lower voltage drive signals for the piezoelectric elements to maintain similar electric field strength as conventional elements. For example, the piezoelectric elements disclosed herein may drive voltages ranging from around 5V to 40V peak to peak.

The capacitance of the piezo element may also be increased by the reduction in thickness for certain piezoelectric materials. Thus, as an example, when drive voltage reduces from 100V to 10V when driving ×10 times thinner film, capacitance may increase by ×10 for the thinner piezoelectric materials, and power dissipation may be reduced by a factor of 10. This reduction in power dissipation also can reduce heat generation and temperature rise in the imaging probe. Thus, using lower drive voltages, the temperature of the pMUT surface can be lowered.

In some embodiments, for a given temperature, when using low voltage pMUTs, more pMUT elements can be driven to illuminate a larger area. This may allow faster scanning of the target, especially if multiple emissions are needed to scan the entire target to form an image. Often, the target area may be scanned with multiple emissions using different steering angles and image data combined to obtain a higher quality image.

It may also be desirable to image at a high frame rate. A frame rate measures how many times a target is imaged per minute. It is desirable to image at a high frame rate when tissue motion is involved to observe targets moving without image blurring. In some embodiments, the ability of driving more piezoelectric elements can allow more coverage of the transducer aperture per emission, minimizing the number of emissions needed to cover the entire aperture, thus increasing frame rates.

In some embodiments, image quality can be improved by compounding several frames of images into one resultant lower noise frame. However, this can reduce frame rate. When using low power pMUT with higher frame rate compared with that of the conventional piezo films, for a given rise in pMUT temperature, this averaging technique can be used due to low voltage pMUTs having lower power thus enabling inherently higher starting frame rate. In some embodiments, synthetic aperture method of ultrasound imaging can be used to allow compounding of images.

In some embodiments, ability to drive more piezoelectric elements at a time improves signal to noise ratio (SNR) and enables better quality of the reconstructed image.

Further, as noted in FIG. 1, an ASIC 106 is coupled to the pMUT 102. The ASIC can contain low noise amplifiers (LNA). The pMUTs are connected to the LNA in receive mode through switches. The LNA converts the electrical charge in the pMUT generated by a reflected ultrasonic beam exerting pressure on the pMUT, to an amplified voltage signal with low noise. The signal to noise ratio of the received signal can be among the key factors that determine the quality of the image being reconstructed. It is thus desirable to reduce inherent noise in the LNA itself. This can be achieved by increasing the transconductance of the input stage of the LNA. This can be achieved for example by using more current in the input stage. More current may cause power dissipation and heat to increase. However, in cases where low voltage pMUTs are used, with ASIC in close proximity, the power saved by the low voltage pMUTs can be utilized to lower noise in the LNA for a given total temperature rise acceptable when compared to transducers operated with high voltage.

FIG. 3B shows a schematic diagram of a pMUT element 220 disclosed herein. In this embodiment, the pMUT element 220 includes 2 sub elements 220a, 220b. In some embodiments, each pMUT element includes one or more sub elements. Each sub element, in this embodiment, has a piezoelectric layer 221, with a first electrode 223, connected to first conductor 222 and a second electrode 225 connected to a second conductor 227, and a third electrode 224, connected to a third conductor 226, where the first conductor of all sub elements are connected together and second conductors of all sub elements are connectors tied together and all third conductors of all sub elements are connected together.

In some embodiment, the pMUT element 220 includes 2 subelements 220a, 220b, wherein each pMUT element has 2 terminals. For example, 220a has a first electrode 223 connected to first conductor 222 and a 2^{nd} electrode 225 connected to 2^{nd} terminal 227 and 220b has a first electrode 223 connected to first conductor 222 and a 2^{nd} electrode 225 connected to 2^{nd} terminal 227.

In some embodiment, the pMUT element 220 includes 1 subelement 220a where each the pMUT element has 2 terminals. For example, 220a has a first electrode 223 connected to first conductor 222 and a 2^{nd} electrode 225 connected to 2^{nd} terminal 227.

In some embodiments, a sub element 220a can have a multiplicity of sub elements, with each sub element having 2 electrodes, with all first electrodes connected to a first conductor and all second electrodes connected to a second conductor.

FIG. 3C is a schematic diagram of a pMUT element 228 with 2 sub elements 228a, 228b. In some embodiments, each pMUT element includes one or more sub elements. Each sub element, in this embodiment, has a piezoelectric layer 231, with a first electrode 230, connected to first conductor 229 and a second electrode 232 connected to a second conductor 233, where the first conductors of all sub elements are connected together and second conductors of all sub elements are connectors tied together.

In some embodiments, a sub element 228a, 228b, can have a multiplicity of sub elements, with each sub element having 2 electrodes, with first electrode of first sub element connected to another electrode of a second sub element with a conductor and the second electrode of the first sub elements connected to the remaining electrode of the second sub element, for the case where there are 2 sub elements in an element.

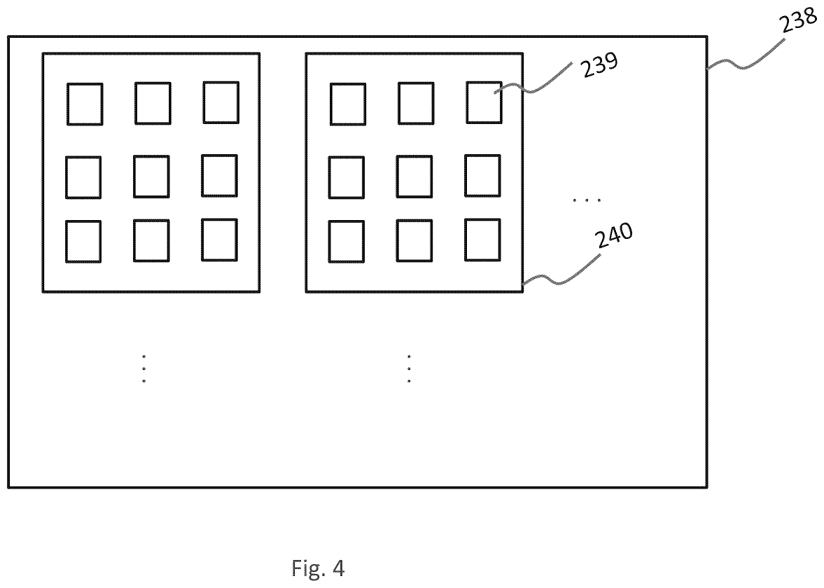

FIG. 4 shows a substrate 238, on which a plurality of piezoelectric micro machined ultrasound transducer (pMUT) array elements 239 are arranged. In this embodiment, one or more array element forms a transceiver array 240, and more than one transceiver array are included on the substrate 238.

Conventional transducer arrays use piezoelectric material, e.g., lead zirconate titanate (PZT) formed by dicing a block of bulk PZT to form individual piezo elements. These tend to be expensive. In contrast, pMUT arrays disclosed herein are disposed on a substrate (e.g., wafer). The wafer can be in various shapes and/or sizes. As an example, the wafer herein can be of sizes and shapes of wafers in semiconductors processes used for building integrated circuits. Such wafers can be produced in high volume and with low cost. Exemplary wafer sizes are: 6, 8 and 12 inches in diameter.

In some embodiments, many pMUT arrays can be batch manufactured at low cost. Further, integrated circuits can also be designed to have dimensions such that connections needed to communicate with pMUTs are aligned with each other and pMUT array (102 of FIG. 1) can be connected to a matching integrated circuit (106) in close proximity, typically vertically below or proximal to the array by a distance, e.g., around 25 μm to 100 μm. In some embodiments, the combination of 102, 104, and 106 is referred to as an imaging assembly 108 or a tile, as shown in FIG. 1. For example, one exemplary embodiment of the assembly 108 can have 1024 pMUT elements, connected to a matching ASIC that has the appropriate number of transmit and receive functionalities for 1024 piezoelectric elements. In some embodiments, the array size is not limited to 1024. It can be smaller or larger. Larger sizes of pMUT elements can also be achieved by using multiple pMUT arrays 102, along with multiple matching ASICs 106, and assembling them adjacent to each other and covering them with appropriate amounts of impedance matching material 104. Alternately, a single array can have large number of pMUT elements arranged in rectangular arrays or other shapes with a number of pMUT elements ranging from less than 1000 to 10,000. The pMUT array and the plurality of pMUT elements can be connected to matching ASICs.

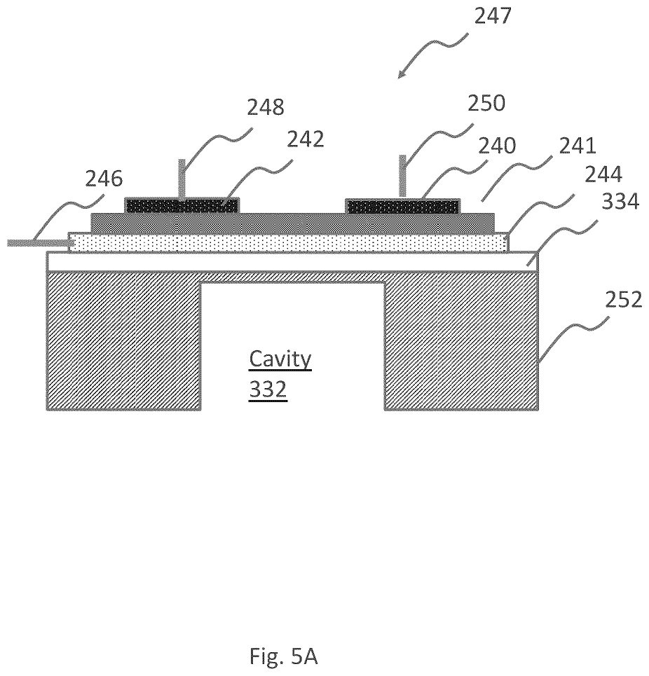

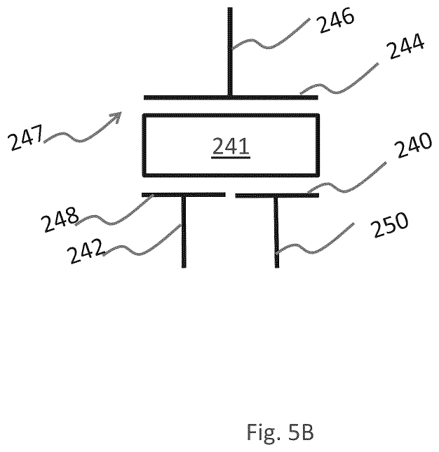

FIG. 5A shows a cross section of an exemplary embodiment of a piezoelectric element 247. In this embodiment, the element 247 has a thin piezoelectric film 241, disposed on a substrate 252. The piezo electric film has a first electrode 244 that is connected to a signal conductor 246. This electrode is typically deposited on the substrate on which SiO2 is grown. A layer of TiO2 followed by platinum is deposited on which PZT is sputtered or a PZT sol gel is applied to develop a thin layer of PZT as the piezoelectric film 241. This and the first metal electrode are patterned by etches to the shape desired. The signal conductor 246 is connected to the first electrode. A second electrode 240 is grown above the thin film 241 and connected to a second conductor 250. A third electrode 242 is also grown adjacent to the second electrode but electrically isolated from it. A third conductor 248 is connected to the third electrode. The actual layout of the electrodes shown can vary from square to rectangular, elliptical and so forth adjacent electrodes or annular electrodes, with an electrode surrounding another. The piezoelectric film can have different shapes and can be present in certain portions over the substrate and cavity.

FIG. 5B is a symbolic representation of the piezo element of FIG. 5A. In some embodiments, the first conductor 246 is electrically connected to the first electrode 244. Such connection may be using metals, vias, interlayer dielectrics (ILD) and these are not shown for simplicity. The first electrode is in contact with the piezoelectric layer 241. The second conductor is deposited or grown on the other side of the piezoelectric layer, with respect to first electrode. The second electrode 248 is connected to a second conductor 242. A third electrode 240 is located adjacent to electrode 248 and a third conductor 250 connects to it. The first electrode 244 is also referred to as an “O” electrode. The second electrode is referred to as the “X” electrode and the third electrode is referred to as the “T” electrode. It is understood that in the interest of simplicity, connection means to connect the conductors to the electrodes such as use of vias, interlayer dielectric (ILD), and other metal layers are not shown in all Figures or discussed in detail. These details are well known to those versed in the state of the art. Also, other details such as showing underlying membranes are not shown.

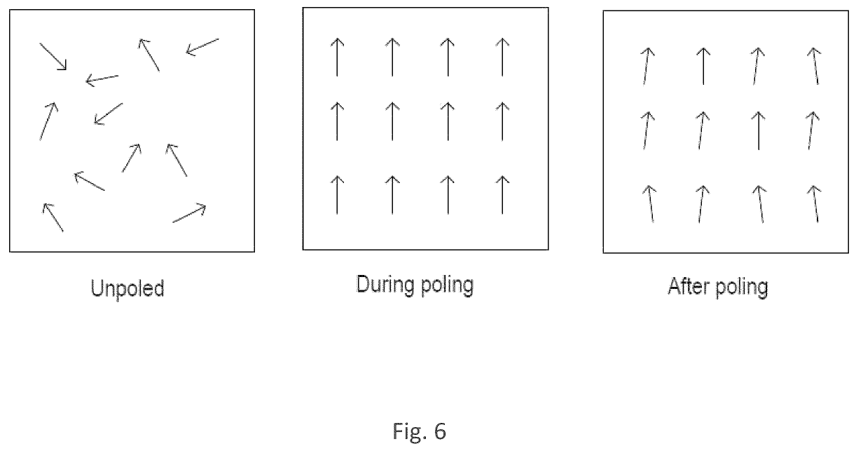

Due to non-symmetry in the crystalline structure of PZT, an electrical polarity develops, creating electric dipoles. In a macroscopic crystalline structure, the dipoles by default can be found to be randomly oriented as shown in FIG. 6 on the left. When the material is subjected to a mechanical stress, each dipole can rotate from its original orientation toward a direction that minimizes the overall electrical and mechanical energy stored in the dipole. If all the dipoles are initially randomly oriented (i.e., a net polarization of zero), their rotation may not significantly change the macroscopic net polarization of the material, hence the piezoelectric effect exhibited may be negligible. Therefore, it is important to create an initial state in the material such that most dipoles may be more-or-less oriented in the same direction. Such an initial state can be imparted to the material by poling it. The direction along which the dipoles align is known as the poling direction. Orientation of dipoles during and after poling is shown in FIG. 6 (middle and right panels).

Piezoelectric thin films therefore may need to be poled initially before being used. This can be done by applying a high voltage across the film, typically at high temperature (e.g., 175° C.) for some time (e.g., 1-2 minute or more). In the piezo element of FIG. 3, a pMUT can be built with 2 terminals and a high voltage can be applied for example across 216 and 218. This high voltage can be around 15V for a 1 μm thick piezo film. Such a voltage is sufficient for poling.

Prior art pMUTs or other piezo elements from bulk PZT typically have two electrodes. As disclosed herein, the piezoelectric element may have 2 (in FIG. 3) or more electrodes, as shown in FIGS. 5A and 5B. In FIGS. 5A and 5B, the first conductor during poling can be tied to ground potential, while the second conductor is tied to a negative potential, say-15V for a 1 μm thick PZT film, and the third electrode is tied at +15V for some time at high temperature. This can create 2 poling directions across the PZT film that are opposite for film between first and second conductors vs piezo film between first and third conductor. After poling is complete, during transmission or receiving operation, the second and third conductors can be connected to ground or a bias voltage, while the first conductor is connected to an ASIC to be driven by a transmit driver during transmit operation or is connected to an LNA through switches during receive operation. The second and third conductors can also be tied to a non-zero DC bias, where the bias values can be different.

The piezoelectric element in an exemplary embodiment utilizes transverse strain, leveraging PZT transverse strain constant d31, the piezoelectric coefficient, to create movement of a membrane or convert movement of a membrane into charge. The PZT element of FIGS. 5A and 5B with orthogonal poling directions to the film in transmit operation amplifies movement of the membrane for a given drive compared to a structure shown in FIGS. 3A and 3C with only one poling direction for the film. Thus, transmit sensitivity can be improved, allowing larger movement of the membrane per volt of applied transmit drive.

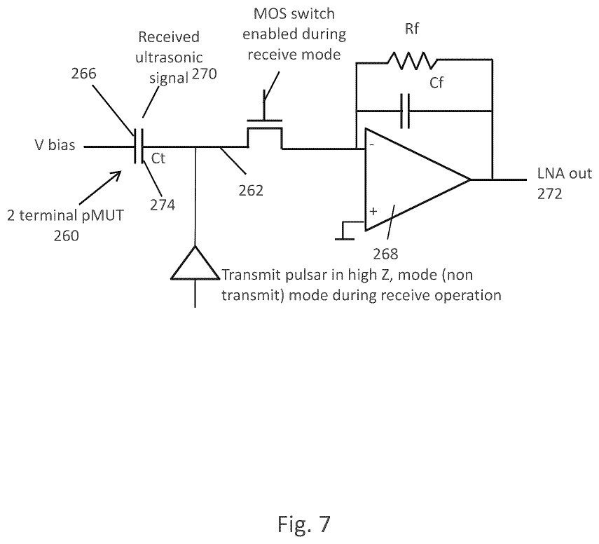

In receive mode, the orthogonal poling direction, may create more charge to be sensed by a LNA. The LNA connections are shown symbolically in FIG. 7. Not all elements in the path of connecting the piezo element to the LNA are shown for simplicity. Piezoelectric element 260, in certain embodiments, has a first electrode connected to a switch in series with LNA 268, connected by conductor 262. The second electrode of 260 is 266 and may be connected to a DC bias that includes 0 V (ground). 270 represents a reflected ultrasonic beam striking the pMUT element 260 and creating charge across electrodes 266, 274. It is to be noted that the LNA can be designed to operate in voltage or charge mode. pMUTs may tend to have large capacitance and for a given amount of charge will create a lower voltage across the transducer than for PZT bulk elements with much smaller capacitance if voltage sensing is used, where the voltage on the transducer is amplified. Since the voltage at input of LNA is small, output is noisier. Charge amplification can provide better signal to noise ratio at the output of the LNA due to high capacitance of pMUT elements compared to voltage mode operation, especially when pMUTs produce more charge output for a given input pressure in receive mode. This is explained in FIG. 7, where any charge received by Ct is transferred across a much smaller capacitor Cf creating a bigger voltage at output of LNA. These LNAs can also be designed so that they power up or down rapidly (e.g., in less than 1 μsec).

Traditional 2D imaging is done using columns of elements that are designed in tall rectangular shape. Alternately, this can be achieved by taking many smaller elements arranged in a column. Individual array elements may be combined to act as a single larger 1D array element to make up a column. This is achieved by hardwiring these individual elements, to create a larger element which has one signal conductor and a common ground conductor. Transmit drive, receive sense and control are implemented for this one combined and larger two-lead pMUT.

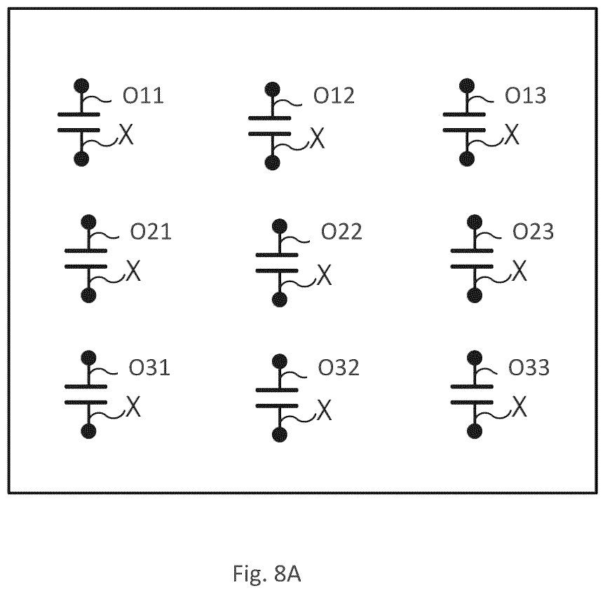

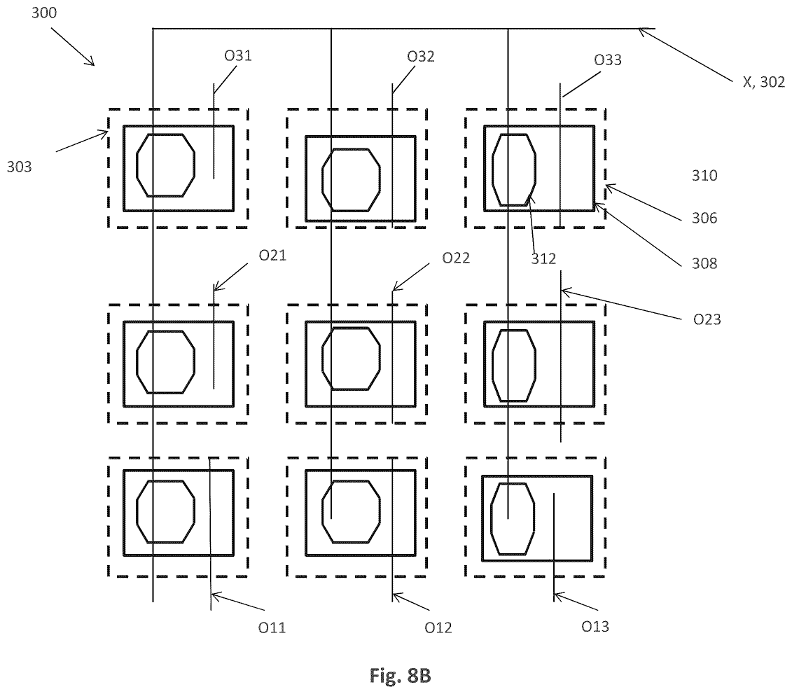

FIG. 8A shows the schematic diagram of an exemplary embodiment of the ultrasonic imaging array 300 of a transducer herein. The array is shown with 9 pMUT elements, arranged in 3 rows and 3 columns or 3 by 3, for the purpose of illustration. It is understood that in practice the array size can be various sizes larger or smaller as needed. Non-limiting example of the sizes include: 32 by 32, 32 by 64, 32 by 194, 12 by 128, 24 by 128, 32 by 128, 64 by 128, 64 by 32, 64 by 194 (columns by rows or rows by columns).

Symbolic representation as used in FIG. 5B is used here for this array of pMUTs. The conductor of each piezoelectric element is connected to the electrode and is named Oxy, where x ranges from 1 to 3 and y ranges from 1 to 3, in FIG. 8B. The first conductor of each piezoelectric element is connected to the first electrode and is named O11. Further, all elements for an electronically configurable imager have their O leads connected to a corresponding electronics located on another wafer. The second electrodes of each element, called X, are all connected to other X electrodes for the other elements by conductor 302. The conductor O is a signal conductor, while the X is a ground or bias line. In this embodiment shown in FIG. 8B, the O electrodes are connected to an ASIC in close proximity to the substrate on which pMUTs arc disposed. In an exemplary case where there is an array of 32 by 32 pMUTs, there are 1024 piezo elements. There can be 1024 “O” lead connections to the ASIC, typically located below the pMUT die. Each of these 1024 O lines are connected to a transmit driver during transmit operation and to the input of a LNA during receive operations, where the transmit driver goes into a high impedance state in the receive mode.

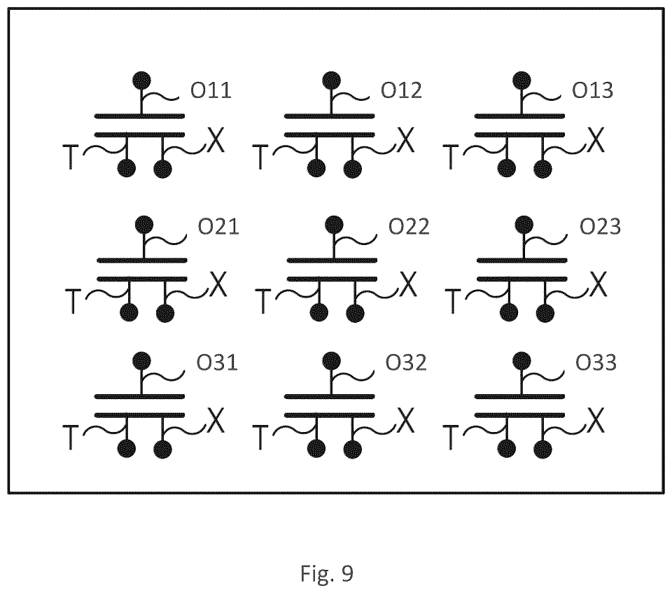

FIG. 9 shows an exemplary embodiment of a transducer array with 3×3 elements, where each element has 3 leads/nodes, i.e., O, X and T. The O nodes are shown as Oxy, where x ranges from 1 to 3 and Y ranges from 1 to 3. These O nodes can be connected to drive and sense electronics in the ASIC, where the X nodes can be connected together to a bias supply or ground and T nodes can be connected together to another bias supply or ground.

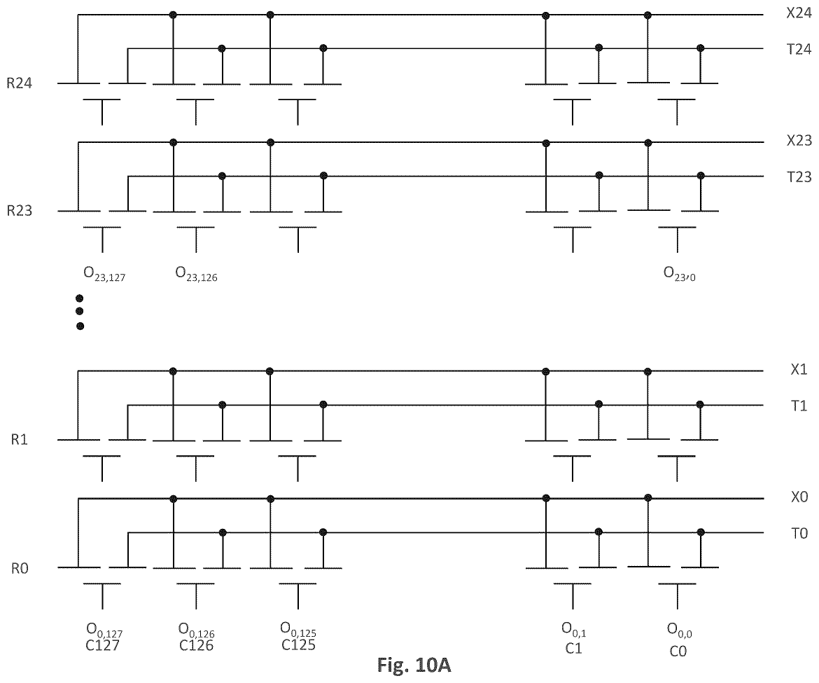

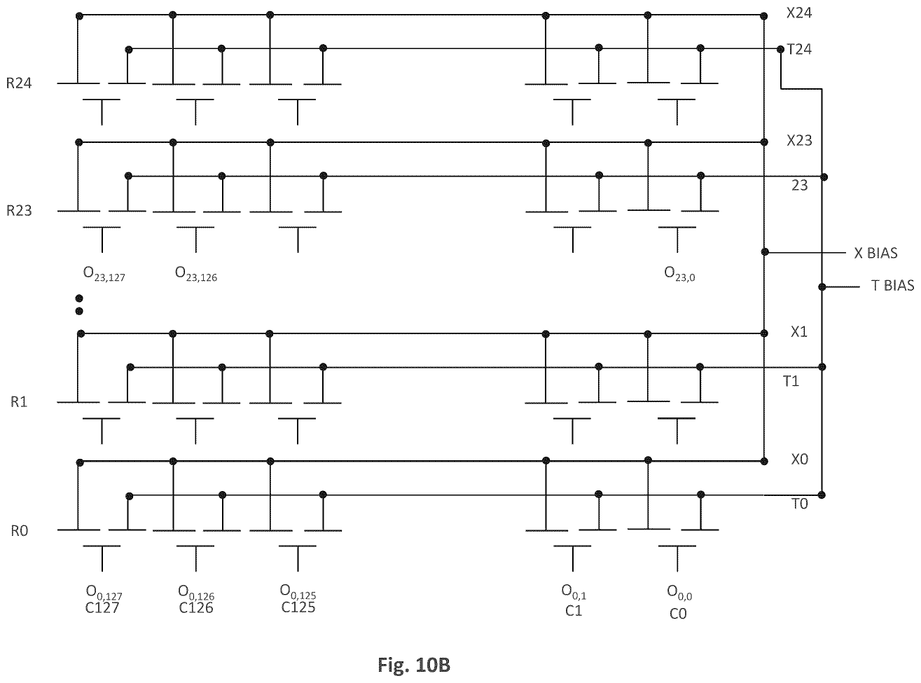

FIG. 10A shows an exemplary pMUT array in which each pMUT element is with 3 terminals. In this embodiment, the array is with 24 rows where each row is made up of 128 elements. Similarly, in the same embodiment, each column can be made up of 24 elements, where all elements may have 3 terminals named O, X and T. For example, the O electrode for the element at the lower left corner is labeled O_{0,127}. This element can have 2 other electrodes namely X and T. Note all elements on a row can be connected to conductor X0 and all T terminals can be connected T0. During a poling operation, all O terminals can be connected to 0V; all X terminals can be connected to a negative potential −V0; and all T terminals can be connected to a positive potential +V0. For the next row, the potentials for row 1, can be higher-V1 for X1 and +V1 for T1 till max voltage are applied for row R11 with voltages V11, −V11. The voltages for the top half of rows can be symmetric to the bottom half. Under these bias conditions, the circuit may be poled at a high temperature of around 175 C. FIG. 10B shows that after poling for imaging use, all X terminals may be connected together and connected to bias voltage as is also the case for all T terminals. Note bias voltages for X and T can be different. In this arrangement, due to differential poling along the column, apodization can be achieved in the elevation direction, where side lobes leakage may be minimized in the elevation plane. In some embodiments, the transducer array can also include only 2 terminals per element, e.g., an X terminal and an O terminal, the T terminal may not be used.

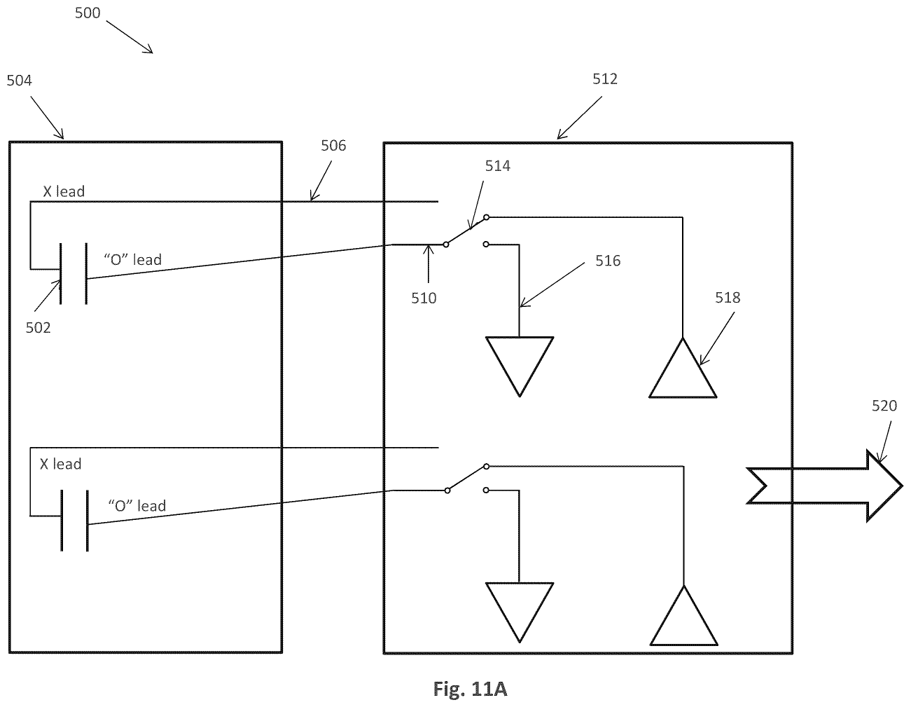

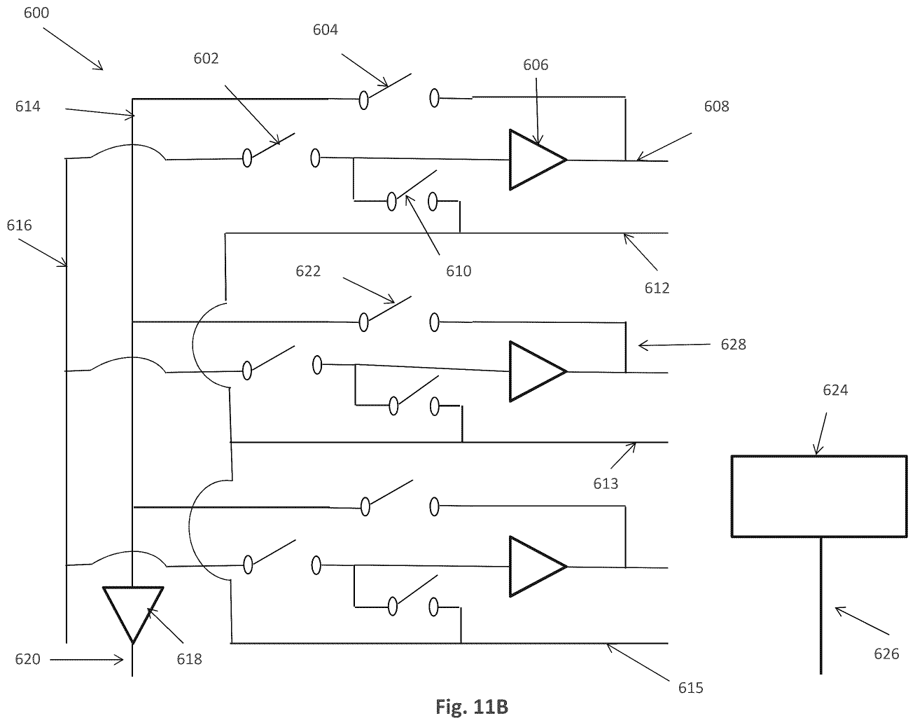

FIG. 11A is a schematic representation of interconnection of 2 transducer elements to an ASIC 500. In certain embodiments, the 2 transducer elements 502 are on one substrate 504, to an ASIC containing transmit and receive and other functions on another substrate 512. Input of a LNA 516 is connected by switch 514 to lead 510 which connects it to the signal conductor of the transducer, the O lead. In some embodiments, bias conductors 506 are connected into the ASIC and later come out of the ASIC for connection to ground or other biasing voltages. These are the X leads of the transducers and may be connected together with other X leads in the transducer and the ASIC. A transmit driver 518 may be controlled by communication external to ASIC on substrate 512, as indicated by 520. It may also be connected to switch 514, which shows switch connection when in transmit mode. The output of the LNA and the input of the transmit driver as shown in FIG. 11A may require 2 different leads. It is possible to use one lead, by using a multiplexer switch similar to 514. In some embodiments, connection to LNA output can be provided to outside electronics in receive mode, and input to transmit driver can be provided in transmit mode.

FIG. 11B shows a schematic representation of some of the functionality in the ASIC for one column of electronics. Functionally, the one column of electronics may interface directly to one column of pMUTs to constitute a composite larger line element. It is understood that the ASIC may contain circuitry for other columns or rows and include other supporting circuitry not shown. It is also understood that the actual functionality desired can be achieved with a different circuit topology that would be considered obvious to those familiar to the state of the art. The representation shown is simply to illustrate the idea itself.

FIG. 11B shows an exemplary schematic of one column of an ASIC 600. In certain embodiments, a conductor 608 is connected to a corresponding signal conductor for an element in the pMUT array of FIGS. 8A-8B, O31. Similarly, O21 of FIGS. 8A-8B is connected to 628 of FIG. 11B. A transmit driver 606 can be connected to the conductor 608 in FIG. 11B. This driver 606 may have a switch 602 connected to its input and connects to lead 616 (the signal conductor for the line element) that connects to the input of other transmit drivers in that column through switches on that column. The switches can be controlled by control unit 624, which via communication with an external controller, may determine which switch(es) are to be turned on. Signal conductor 616 may also connect to electronics implementing transmit beamformers. The O conductor 608 can also connect to a switch 604; the other side of the switch 604 may connect to similar switches in that column (for example, 622). The line 614 may also be connected to the input of a low noise amplifier (LNA) 618. Only one LNA may be required for each line element (or column). The LNA can be activated in receive mode by the control unit 624, which also turns on switches (e.g., 604), while turning off other switches (e.g., 602). This may connect the signal electrode of the pMUTs (through connection 608) to the LNA, which may amplify and convert the received signal to a voltage output 620 with low additive noise. Note, in receive mode, the controller may also make the transmit drivers go to disabled mode, where their output impedance becomes very high, so as not to interfere with the receive signal. In transmit mode, when a piezo element is not supposed to transmit, switch 610 can be turned on, with switches 602 and 604 off, to ensure a net-zero volt drive across the pMUT signal and bias electrode for elements that are not supposed to transmit signals while in transmit mode. The X lines are also connected to the ASIC. Note, in FIGS. 8A-8B only 1 biasing electrode X is shown. But multiple biasing electrodes can exist. For example, FIG. 9 shows an implementation with 2 biasing electrodes X and T. In principle, only 2 connections are needed for the entire array to connect to the T and X electrode, but it is desirable to have many more to achieve high quality imaging. Increasing the number of connections for T and X between the ASIC and pMUT reduces impedance in the X and T conductors when connected in parallel to ground or bias sources, and this reduces crosstalk. Crosstalk is the coupling of signals from an imaging element to another one, creating interference and image quality reduction. Spurious electrical coupling can be created when any voltage drop due to current flowing in X and T lines appears across a piezo element that ideally should not be exposed to that voltage. When the piezo element is not transmitting or receiving under electronic control, the X, T, and O electrodes are locally shorted.

For simplicity, FIG. 11B only shows the connection to only one of the 2 biasing conductors (X in FIGS. 8A and 8B). But it is understood that means for connecting both X and T terminals also exist, to support pMUT arrays similar to that shown in FIG. 9.

In some embodiments, conductor 612 in FIG. 11B can be connected to X, 302 in FIG. 8B. In some embodiments, conductor 613 in FIG. 11B can also be connected to X, 302, but at locations closer to 613, and so on. Note, these additional interconnections 613 and 615 are not essential, while at least one connection (either 612 or 613 or 615) is needed. FIG. 11B also does not show circuitry needed to connect to T electrodes of FIG. 9. The circuitry needed can be similar to that used for connection to the X electrode.

FIG. 11B shows that the receive output 620 and transmit input 616 may require 2 leads. But using a multiplexer, one lead can also be used for this purpose.

A line imager herein may include a multiplicity of piezo element columns, each column is connected by at least a signal and bias leads to a controller. Pulses of an appropriate frequency drive a line. The other lines are driven with delayed versions of this pulse. The amount of delay for a certain line is such that it allows the resultant beam transmitted to be steered at an angle or be focused at a certain depth, with operations known as beamforming.

The line imager of FIGS. 8 and 9 are electronically configurable. Using an example of an array of piezo elements that are arranged with 24 elements in one direction and 64 elements in an orthogonal direction (azimuth direction for this example), a 64 line imager can be built, with each line consisting of up to 24 elements. However, the size can be electronically adjusted from 0 to 24 elements for any line and any number of lines in azimuth up to 64 can be activated

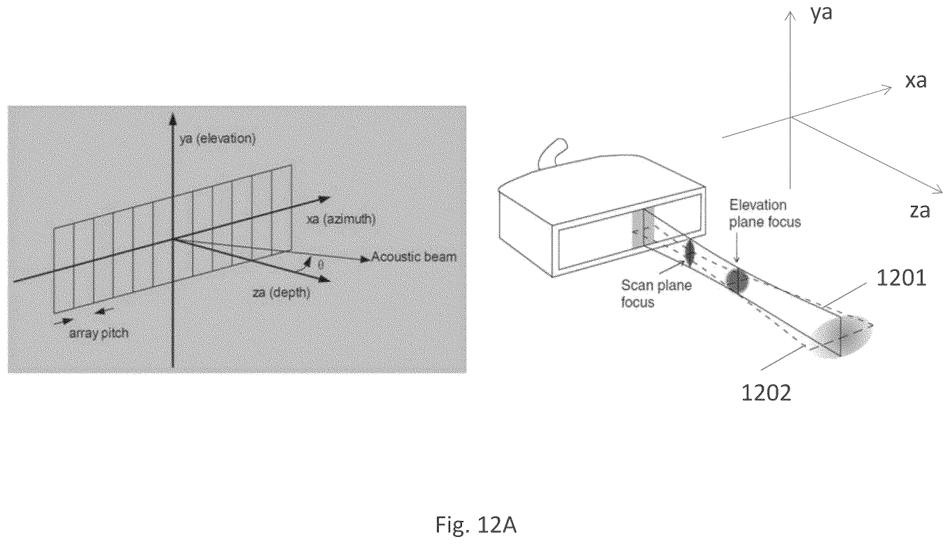

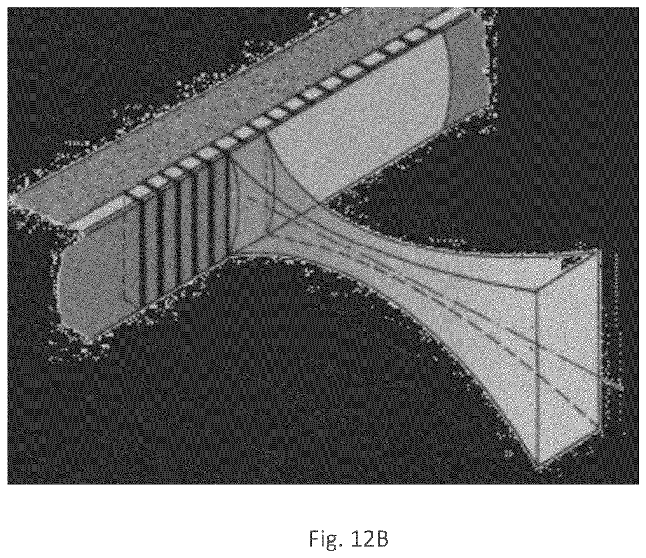

It is desirable in a 2D or 3D imager to image a thin slice of the elevation plane as shown in FIGS. 12A and 12B. In this particular embodiment, elevation direction is in the ya axis on the left panel. An elevation plane 1201 is within the ya-za plane. In the same embodiment, the azimuth plane 1202, also the scanning plane herein, is orthogonal to the elevation plane. Referring to FIG. 12B, a mechanical lens focuses beams in the elevation plane, keeping beams from straying away to form a much thicker slice in the elevation plane and hitting other objects in the thicker elevation slice with the unwanted reflections becoming part of the received signal, adding to signal cluttering and degrading the image quality.

If the beam spreads well beyond the intended slice thickness, it could potentially hit targets outside the desired range and reflections from those will create clutter in the reconstructed image. A mechanical lens formed on the transducer surface can focus beams in the elevation plane to a fixed elevation slice thickness as seen in FIG. 12B, where thickness is minimum at elevation focus point as seen in FIG. 12B and also noted on FIG. 12A as elevation plane focus point. Electronic focus for 2D imaging will allow for improved focus in the elevation plane by virtue of dynamic receive focus as a function of time. Here, the focal length in elevation is varied as the beam travels down toward the target and results in a superior image. For 3D imaging, a fixed mechanical lens does not work, since that specific elevation slice cannot be steered or sweep over the desired volume. Therefore, an electronically controlled elevation focus is desirable.

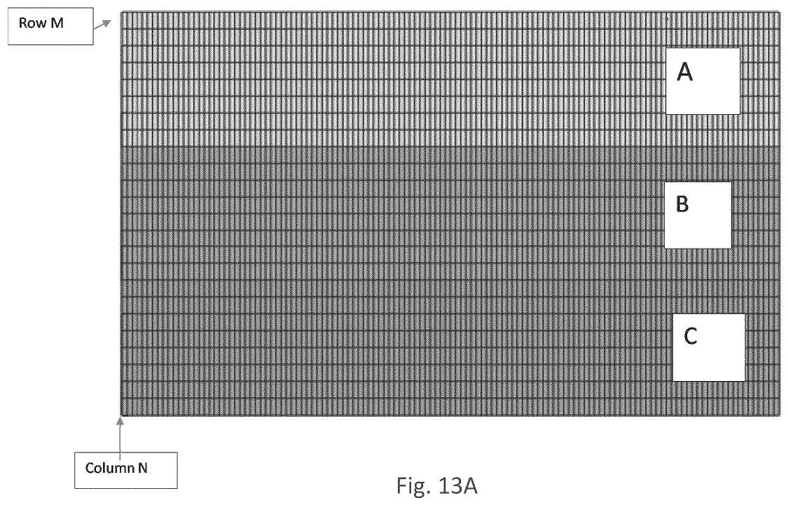

In some embodiments, this is achieved by dividing the transducer into a number of different strips. Referring to FIG. 13A, in a particular embodiment, the transducer with multiple transducer elements is organized in N columns, where each column has up to M rows of transceiver elements. The rows of elements can be divided into strip A which includes a first number of rows, where the strip A has up to N columns, strip B that includes a second number of rows that are in the central section of rows with each row of up to N columns, and strip C that includes a lower section of rows of up to N columns. In some embodiments, each of the strips can be selected to be driven separately and where columns in each strip share the same drive by transmit driver(s). The strips A, B, and C can be non-overlapping with adjacent strip(s). Alternatively, the strips may overlap for a number of rows and columns with its adjacent strip. In some embodiments, the strips together cover all the N columns and M rows of the transducer element. In some embodiments, when electrically programmed, the strips all together may only cover a part of the M by N array of the transducers.

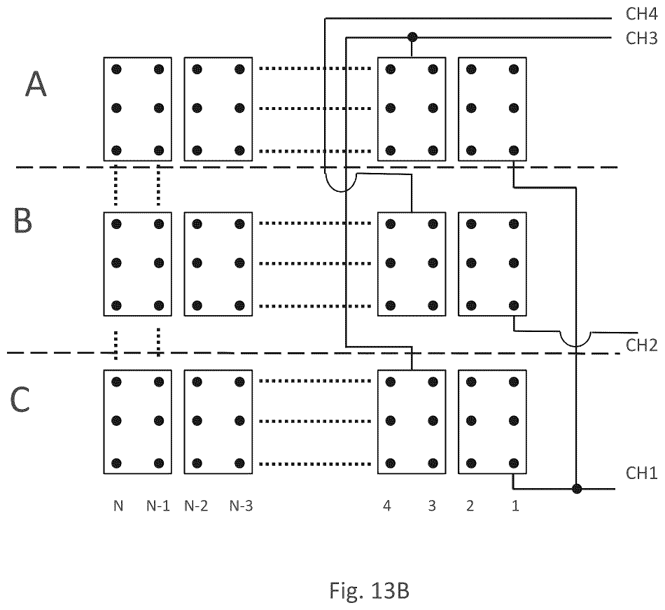

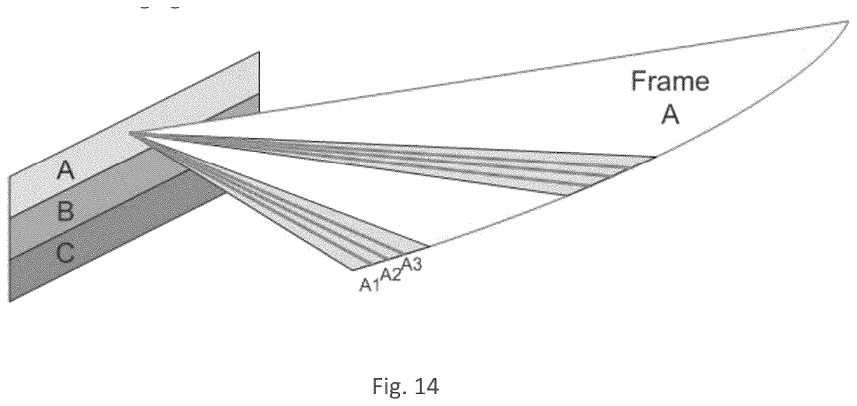

In some embodiments, the top section A is organized such that all elements in that section are driven by transmit driver(s) intended for the column that the element(s) are in. In this embodiment, in a transmit operation, N transmit driver with unique delays driving N composite columns (each composite column may include elements from rows from strip(s) A or B or C) are used to focus an ultrasound beam in the azimuth plane 1202. During receive operation, reflected signal that impinges in section A is beamformed to create scan line A1, A2, A3, etc. as shown in FIG. 14. Referring to FIG. 14, three strips of PMUTs are labeled as A, B and C. These strips include rows of PMUTs that, where elements on a column are driven by a common transmit driver, have N drivers for N columns (i.e., a different driver for each of the N columns). Scan lines A1, A2, etc. can be formed by transmission and reception using strip A. Scan line B1, B2, etc. are formed from section B and scan lines C1, C2, etc are formed from section C. Now using scan data from the 3 sections, another focus this time in the elevation direction is performed using unique delays to data from section A, B and C in a similar technique that was used to focus beams in the azimuth plane earlier using delays along column drivers. This process may be thought of as a dual stage beamformer, where the first stage consists of developing scan lines from A, B, C and a second stage uses that data to develop focus in the elevation plane. The focus in elevation is achieved in the receiver by digitally applying delays. This technique not only allows a focus in the elevation plane, but also allows focus to be dynamic. In this case, the focal length can be adjusted as a function of time, to allow elevation focus to travel with the ultrasonic beam.

Although the process described in FIGS. 13A and 14 may require three transmissions and receptions, the first and second transmission and reception from section A and C can be combined into one operation. In some embodiments, transmission both from a top portion and a bottom portion of the transducer can be performed simultaneously, where the delays on the top portion and bottom portion of a column are identical. The second transmission is from the central portion with different delays from that used in the first and/or the second transmission.

In some embodiments, the top section, the central section, and/or the bottom section can be divided into one or more subsections, each of which include a number of rows for pulse transmission and signal receiving. In some embodiments, each subsection can be used to form multiple scan lines similar as disclosed herein.

In some embodiments, the transducer element array can be divided into more than 3 strips, for example, 4, 5, 6, 7, etc. In some embodiments, scan lines in each strip can be performed cither sequentially or simultaneously. In some embodiments, in simultaneous transmission, scan lines from strips symmetrical to the center strip are obtained. In some embodiments, the delays for elements in same column are identical for sections operated on simultaneously.

The elevation focus can also be assisted by further employing a lower amplitude of voltage for a part of the two outer sections of the transducer with respect to remaining parts of the transducer.

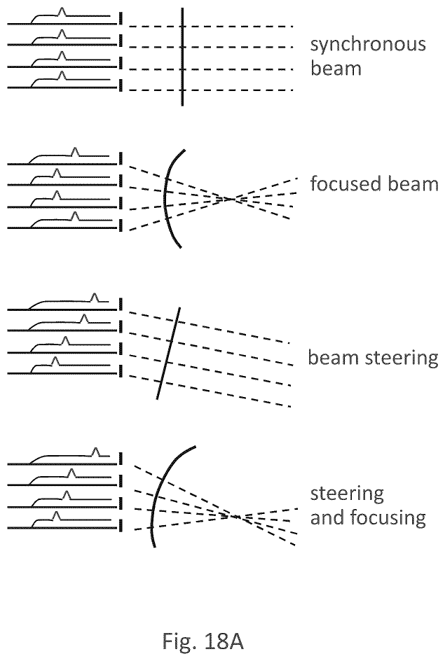

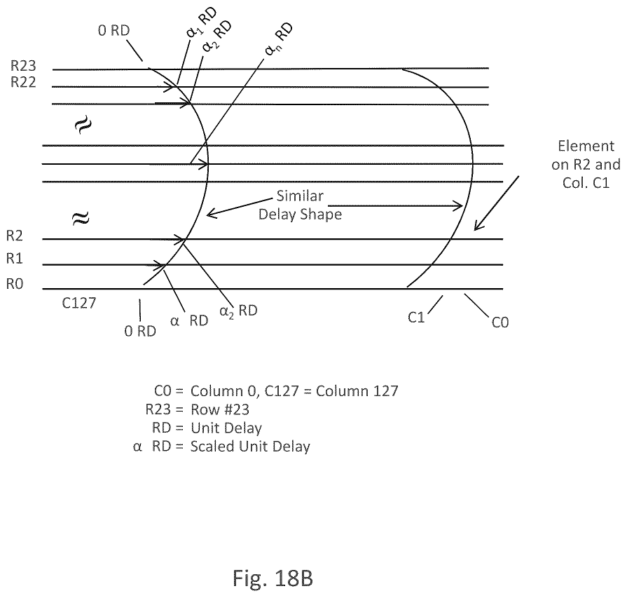

In some embodiments, a unique programmable delay along the elevation direction is implemented for each element of all columns. Assume all N columns receive drive signals that are delayed relative to each other. Additional delays can be generated to add further delay along the column elements, where each element along the column can be delayed differently relative to its adjacent neighbor(s) on the same column. A delay profile example is shown in FIG. 18B. The delay for all columns elements along the elevation direction can be similar. In one embodiment, the delay is symmetrical, with maximum at the center element for a focus in the elevation plane. The amount of delay difference between the outer and central elements determines the focal length.

In some embodiments, the delay profile is shown in FIG. 18B, where relative delay at the edge elements for a column may be 0×RD or 0 ns. For element on row 1 and R22, delay relative to delay at row 0 can be alpha1×RD and so on as shown in FIG. 18B, if symmetric delays are desired around a central element. Delay RD is programmable as are alpha1, alpha2, etc. Therefore, a delay profile can be made up along a column, where the delay can be relative to delay at the edge of the columns. It is to be noted that the relative delay profile can be identical for other column elements. In other embodiments, the delay profile may not be symmetric around a central element and can be arbitrarily programmed. In some embodiments, the delay is in the range of 25 ns to 1000 ns. In some embodiments, the delay is programmable with different ranges of 10 ns to 5000 ns. In some embodiments, the delay is in the range of 50 ns to 500 ns.

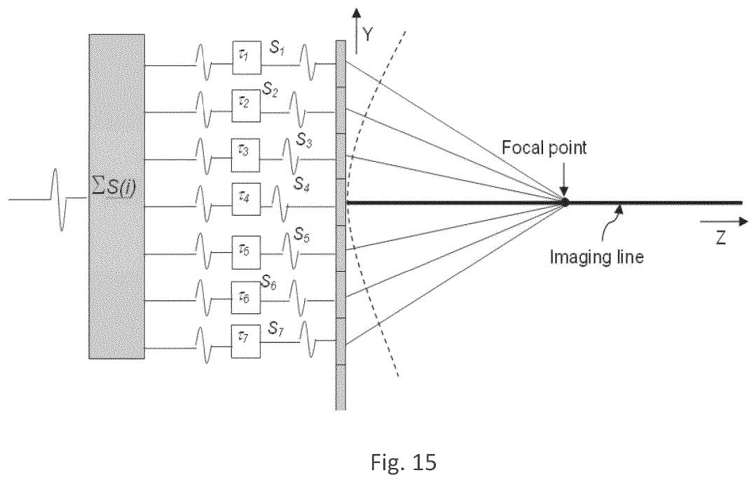

The procedure to obtain a scan line using the systems and methods herein, in some embodiments, is shown in FIG. 15. In some embodiments, the reflected signal is received by the transducer, the signal is converted to voltage and amplified and digitized by an analog to digital converter (ADC). These received signals are also known as RF signals. These RF signals can be delayed by in (e.g., t1, t2, t3, t4 . . . ) and summed to form a scan line, e.g., A1, A2, etc. in FIG. 14. In some embodiments the signals are delayed and weighted with coefficients and then summed to form scan lines.

In some embodiments, focusing a beam in the receive direction utilizes more than one RF signals, e.g., S1, S2, etc. along the azimuth direction (Y), which are digitized output samples known as RF signals. In some embodiments, the RF samples are delayed, for example, with a delay profile along the Y direction, and the resulting signal can be weighted and summed to form a scan line

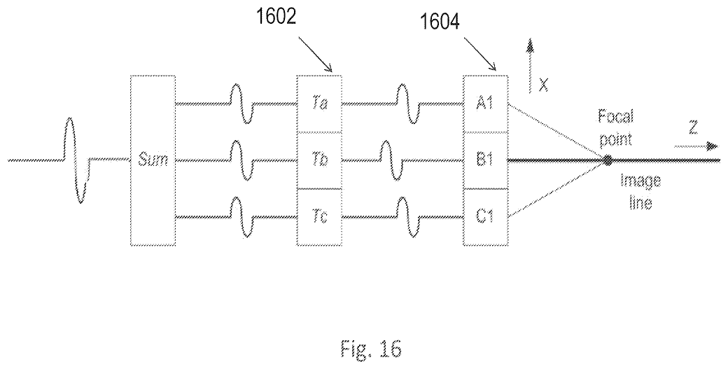

As illustrated in FIG. 14, in successive transmission and reception events, scan line A1, A2 and additional scan lines can be obtained using section A. In some embodiments, an image frame may include many scan lines such as 100 or even more to achieve fine scan of the target area being imaged. Similar procedure can be used to obtain scan lines using Section B and Section C. The scan lines from sections A,B,C are developed using a first level beamformer, where a beamformer creates scan lines using an algorithm, where in the embodiment described the algorithm used a signal delay and sum method previously described. A synthetic aperture, second level beamformer is then used to achieve focus in the elevation plane, as shown in FIG. 16. In some embodiments, these transmissions are focused on a single elevation angle (0 degree, 10 degrees, 20 degrees, 30 degrees, etc.) thereby reducing out of plane clutter that is not within the elevation plane and obtaining an improved image.

As illustrated in FIG. 14, in successive transmission and reception events, scan line A1, A2 and additional scan lines can be obtained using section A. In some embodiments, an image frame may include many scan lines such as 100 or even more to achieve fine scan of the target area being imaged. Similar procedure can be used to obtain scan lines using Section B and Section C. The scan lines from sections A,B,C are developed using a first level beamformer 1602, where a beamformer creates scan lines using an algorithm, where in the embodiment described the algorithm used a signal delay and sum method previously described. A synthetic aperture, second level beamformer 1604 is then used to achieve focus in the elevation plane, as shown in FIG. 16. In some embodiments, these transmissions are focused on a single elevation angle (0 degree, 10 degrees, 20 degrees, 30 degrees, etc.) thereby reducing out of plane clutter that is not within the elevation plane and obtaining an improved image.

Referring to FIG. 16, in a particular embodiment, second stage focusing/beamformer 1604 uses beam data (i.e., scan line data) from: A1, B1, and C1; A2, B2, and C2; A3, B3, and C3; etc., which are delayed, weighted, and summed to form the final beam output to allow elevation plane focusing. In this embodiment, X is the elevation axis.

Unlike mechanical lenses, as disclosed herein with synthetic lenses, the focal distance can be electronically programmed into the beamformer. In some embodiments, the process may require a number of transmissions and receptions (e.g., 1 transmission and reception from N lines to form scan line A1) to form a scan line from any sections of the transducer, e.g., (sections A, B, and C). To form a frame, R scan lines are required to scan the full area to be imaged. Further, in this case, 3 separate frames A, B, C are needed. In some embodiments, it is desirable to have a high frame rate in an image. A frame may include many scan lines. However, if the number of transmissions and receptions can be reduced while the same number of scan lines can be developed, then frame rate will be increased. In some embodiments, increased frame rate can be achieved by combining the transmission and reception from two sections (e.g., A and C). Since these regions are symmetrical with respect to a central region, the delays needed for example as shown in FIG. 15 can be identical for regions A and C. By combining these two regions into one combined region to transmit and receive signals from, the frame rate can be increased by 150%. The central portion B may require different delays from the delays used in the first transmission for regions A and C. In some embodiments, scan lines A1, B1, C1 and the like are formed along the azimuth plane. A second beamforming operation can use data from the first level beamformer 1602 and using similar techniques as shown in FIGS. 15 and 16, a focus can be achieved in the elevation plane. In some embodiments, 2D scans can start from one side of a strip, e.g., column N, and completes at the other end, e.g., column 1. Thus, frame A can be obtained by scanning for beam A1, A2, AN . . . in order. By following this sequence for frame B, which is a sequential frame in time to frame A, the target may have moved. To minimize impact of motion artifacts, beamforming can be done by interleaving scan lines for different frames, such as A1, B1, C1, A2, B2, C2 and so on. When A and C are combined such that transmit and receive can be done together, the combined A, C region can be named as D and scan lines as D1, D2, etc. A non-limiting exemplary scan sequence can be D1, B1, D2, B2, etc. This may help minimize sensitivity to movements in the target being imaged.

In some embodiments, electronic synthetic lens offers dynamic focusing and dynamic aperture. For example, in near field, the weights for A and C can be minimal and gradually increase with depth, thus resulting in change in aperture.

In some embodiments, sections (e.g., A and C) are apodized during transmission and reception. Apodization can be achieved by pulse width modulation (PWM) of the Transmit (Tx) drive waveform. An unapodized pulse drive has a nominal pulse width. When pulse width is changed, e.g., reduced, the pressure output from the pMUT can reduced. In some embodiments, apodization is a tapering of weights for elements as they go from the center of the transducer to the edges. This can reduce side lobes and create higher quality images. By applying apodization to the procedure described, signals leaking outside of the elevation plane can be reduced. FIGS. 10A-10B show an exemplary embodiment of apodization implementation in the elevation direction with a pMUT array. Each pMUT in the array is with 3 terminals. In this embodiment, pMUTs can be programmed with varying polarization strength. The pMUT array, e.g., in FIG. 10A, may be poled using different poling directions for piezo material controlling membranes and different poling strength per row. FIG. 10B shows exemplary biasing connections of the array after poling operation. In some embodiments, the same principle herein applies to the pMUTs that use only 2 terminals, e.g., O terminal and an X terminal.

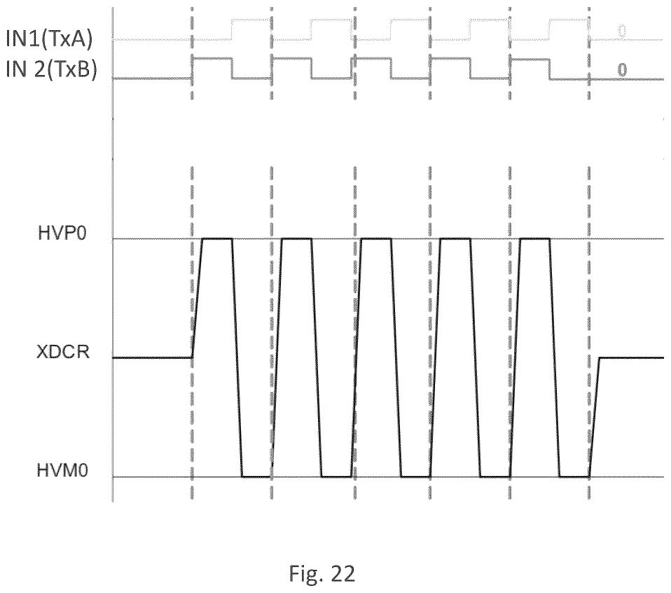

In some embodiments, the apodization can be achieved by using a multi-level transmit drive, for example, 3 or 5 or 7 levels. By choosing different levels of this drive signal, apodization can be created by applying amplitude varying transmit drive signals that are lower in amplitude for elements closer to the edge than the center of the transducer. In this example, all elements on outer rows compared to central rows can have lower drive voltages, and by digital decoding and selecting, certain drive levels may be available to form the multi-level outputs. A three-level decoding example is shown in FIG. 22.

In some embodiments, apodization is implemented by employing piezoelectric elements of smaller size at edges compared to those at the center of the transducer aperture.

In some embodiments, the transducer elements are arranged, as shown in FIG. 13A, in a top section, A, a bottom section, C, and a middle section, B. As shown in FIG. 13B, in each of these sections, i.e., sections A, B, and C, 2 adjacent elements on a row are electrically connected together in a transmit and/or receive operation, essentially converting a N line (equivalently herein to column) transducer to a N/2 line transducer. During transmit operation, every line's top and bottom section can be connected to a channel and the middle section can be connected to another channel. Thus, N channels are needed to service N/2 lines. During a transmit operation, all elements can be operated on and can create maximum transmit pressure by utilizing all elements in the transducer. Focusing in the azimuth direction can be achieved by transmit channels changing the relative delay between lines or columns. During a receive operation, the elements can be connected as shown in FIG. 13B and can be focused in azimuth direction. Elevation focus can be performed using a beamforming operation using the results of the first level beamformer as discussed for FIG. 13A. The transmit and receive operations using transducer elements connected as shown in FIG. 13B can have the advantage of one transmit and receive operation using the entire transducer for maximum signal to noise ratio and fast frame rate. The signal to noise ratio can be higher than the case as shown in FIG. 13A, in which each of the strips can be selected to be driven separately and where columns in each strip share the same drive by transmit driver(s) since all transducer elements are used. Also, motion artifacts can be reduced using the transducer as shown in FIG. 13B rather than using the transducer as shown in FIG. 13A.

In some embodiments, programmable delays can be generated along the elevation direction for one or more columns. In some embodiments, if all N columns receive drive signals that are delayed with respect to each other, additional delays can be generated to add further delay for elements along a same column. In some embodiments, each element along the column can be delayed differently with respect to its adjacent neighbor(s) on the same column. A delay profile example is shown in FIG. 18B. The effective delay for an array element ele_{i,j} can be the summation of the group column delay, τⱼ, and the individual row delay, τᵢ, as following:

$${τ}_{i , j} = {τ}_{j} + {τ}_{i} \qquad ( 1 )$$

where in some embodiments, delay τⱼ, τᵢ, can be determined by:

$${τ}_{j} = \sqrt{{( x - {x}_{j} )}^{2} + {z}^{2}} / c \qquad ( 2 )$$

$${τ}_{i} = \sqrt{{( y - {y}_{i} )}^{2} + {z}^{2}} / c \qquad ( 3 )$$

In equations (1)-(3), the focal point on transmit is at position (x,y,z) and the delays can be calculated independently for the element at position xⱼ, yᵢ. The variable c is the assumed speed of sound in the propagating medium. Note that in the case of perfect, non-separable focusing the delay for transducer element, ele_{i,j}, can be computed as:

$${τ}_{i , j} = \sqrt{{( x - {x}_{j} )}^{2} + {( y - {y}_{i} )}^{2} + {z}^{2}} / c \qquad ( 4 )$$

Note that, in some embodiments, the separability assumption of the delays in azimuth and elevation is not perfect and the largest errors in the delay profile occur on the outer elements of the focusing aperture. However, for embodiments with small steering angles and/or large f/number (where f/number is the ratio of focal length to diameter of aperture) this separability assumption can provide satisfactory results and case of electronic implementation.

The delay for all column elements along the elevation (e.g., same row) can be similar. The delay can be symmetrical, with maximum at the center for a focus in the elevation plane. The amount of delay may determine the focal length.

In some embodiments, a programmable delay along the elevation direction for all columns may be implemented. Assume all N columns receive drive signals that are delayed with respect to each other. Additional delays can be generated to add further delay along the column elements, where each element along the column can be delayed differently with respect to its adjacent neighbor on the same column. Non-symmetrical delays with respect to the center element on a column can also be achieved. In certain embodiments, it is desirable to steer beams in the elevation plane and delays for elements on a column are generated such that each element of a column has a fixed delay increment with respect to its neighbor.

In some embodiments, programmable delays along the elevation direction can be implemented, where the elevation delays can be a summation of two delays, e.g., a coarse, linear delay and a fine, arbitrary delay. Coarse linear delay for elements along a column may be useful, too, for beam steering. To tilt a beam, an element at the bottom of a column may have a lateral delay from an element at the top of the column, where elements in between have linearly interpoled delays. The delays are larger for larger steering angles. Additionally, fine delays along the elements of a column may be useful to focus a beam in an elevation direction. For example, if delays are larger at the center element of a line and delays reduce symmetrically on both sides of the central element, the beam may focus. Small delay values result in beams with larger focal lengths (for example in the tens of ns) and large delay values result in beams with shorter focal lengths (for example in the hundreds of nsec to μsec). If, in some embodiments, all N columns receive drive signals that are delayed with respect to each other, elevation delays can be generated to add further delay along the column elements, where each element along the column can be delayed by two delays, e.g., a coarse and fine delay, where the coarse delay can be linear between adjacent elements and the fine delay can be arbitrary between adjacent elements. The linear delay along the column elements can be different from column to column as well as the fine delay along the column elements can be different from column to column. Therefore, the effective delay for an array element ele_{i,j} can be the summation of the group column delay, τⱼ, the linear coarse row delay, τ_{i,coarse}, and the fine row delay, τ_{i,fine} as following:

$${τ}_{i , j} = {τ}_{j} + {τ}_{i , coarse} + {τ}_{i , fine} \qquad ( 5 )$$

where τⱼ, τ_{i,coarse}, and τ_{i,fine} can be calculated as following:

$${τ}_{j} = ( \sqrt{{( x - {x}_{j} )}^{2} + {( y - {y}_{min} )}^{2} + {z}^{2}} - \sqrt{{x}^{2} + {y}^{2} + {z}^{2}} ) / c \qquad ( 6 )$$

$${τ}_{i , j , coarse} = Δ τ {y}_{i}$$

$${τ}_{i , j , fine} = ( \sqrt{{( x - {x}_{j} )}^{2} + {( y - {y}_{i} )}^{2} + {z}^{2}} ) / c - {τ}_{j} - {τ}_{i , j , coarse} \qquad ( 7 )$$

In equations (5)-(7), the focal point on transmit is at position (x,y,z) and the delays can be calculated independently for the element at position xⱼ, yᵢ. The variable c is the assumed speed of sound in the propagating medium. In equation (6) the yₘᵢₙ parameter can be calculated by projecting the focal point, (x,y,z) onto the 2D transducer plane and calculating the transducer row position with minimum distance to the projected focal point. The slope of the coarse delay, At, can be calculated such that the fine delay can be used to give a good approximation of the perfect 2D delays.

It should be clear to one skilled in the art that the above methodology for calculating delays may give a much better approximation to the 2D focal delays of equation (4) compared to the X-Y separable delays previously mentioned. The improved delay calculation may come at the expense of requiring a coarse delay clock, fine delay clock, and some more register bits for implementing the different delays on a column by column basis. However this method is easier to implement in an integrated circuit than a fully arbitrary delay in two dimensions with fine clock delays and individual element routing.

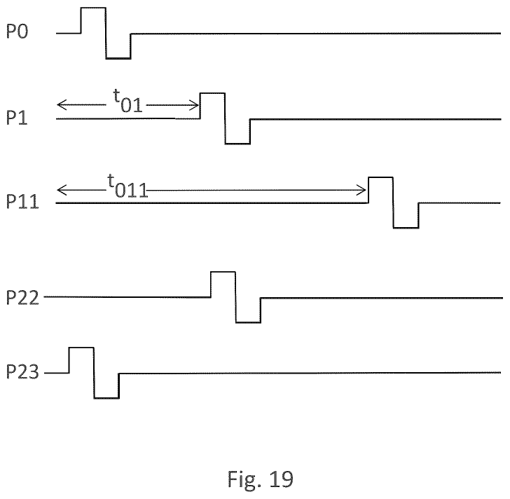

In some embodiments, a cascaded series of flip flops gate a clock arriving at the column from the Tx beamformer with appropriate delay. This delay can then be propagated in the column by a different clock whose frequency is programmable, but synchronized to Tx clock that generated the delay for drivers for the various column drivers. For symmetrical delay around a central element on the column, the flip flop chain generating the delays stops at the central element of the column, where the delay profile can be symmetrical around the center as noted in FIG. 19. The delays generated by the flipflops can be routed to the proper locations so that row 0 element has the same delay as element on the last row, the element on 2^{nd} row has similar delay as element 2^{nd} from last on top side and so on.

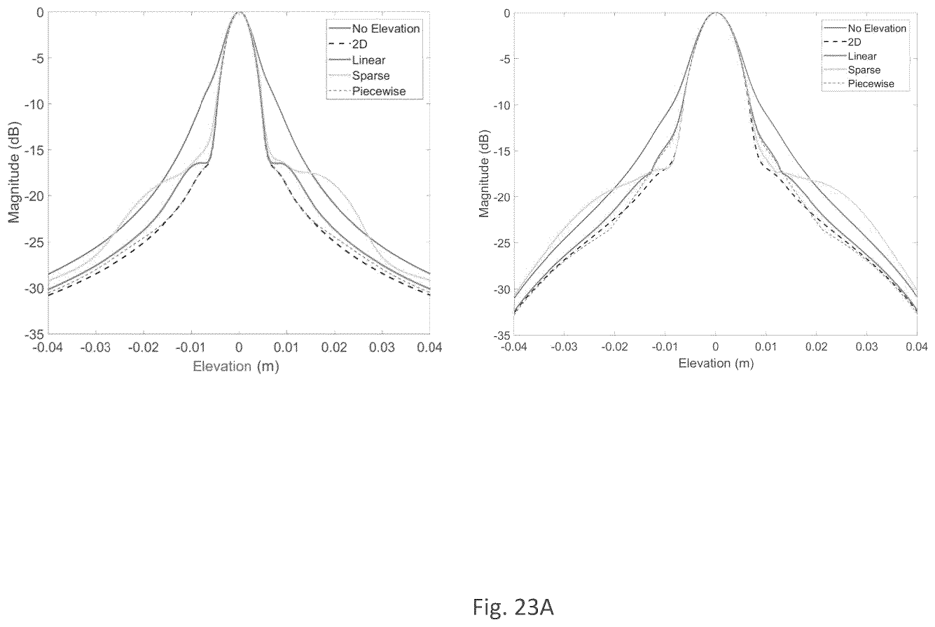

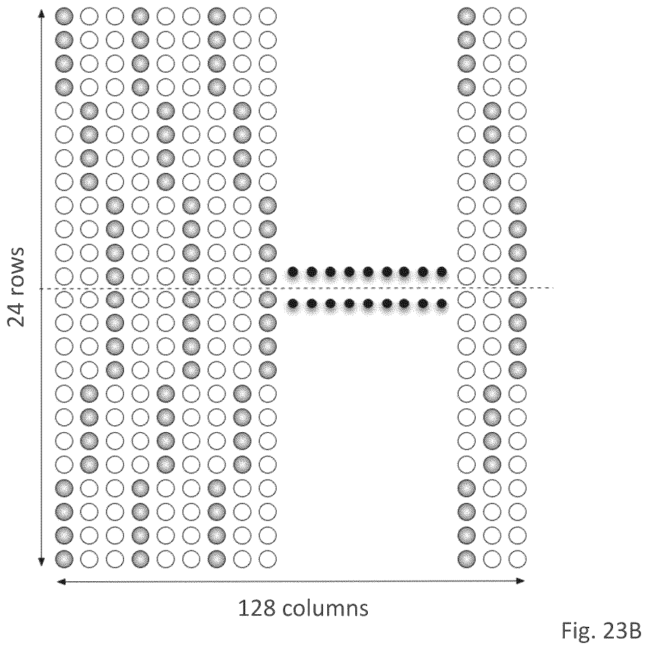

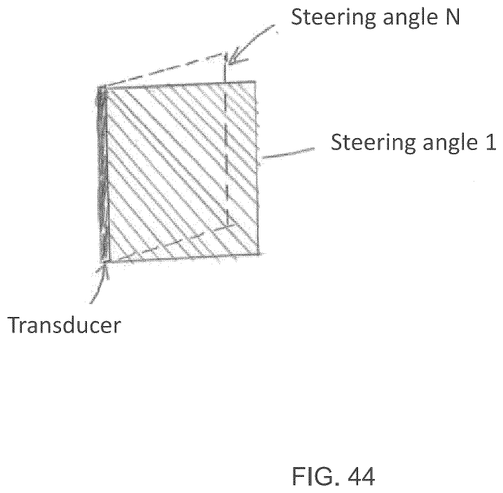

In some embodiments, the delays between adjacent elements in a column can be linear. The results in Table 1 and the elevation beamplots in FIG. 23A shows the effects of using a linear delay profile in elevation compared to a parabolic profile. The results in Table 1 quantify the beamwidths (at −3 dB and −10 dB) of the one-way beamplots in FIG. 23A. As shown in FIG. 23A, in a particular embodiment, five different implementations of elevation focusing are investigated for a 2d transducer array: 1) No elevation focusing 2) Perfect 2D focusing, 3) Linear delays, 4) Piecewise Linear delays and 5) Sparse Apodization. For the linear delay case, the delays between adjacent elements along a column can be fixed with respect to one another, and the elevation delay profile can be symmetric around the center of the array although this condition is not necessary. For the Piecewise linear delays, the delay profile can be separated into at least 3 segments where the adjacent elements in a given segment have a fixed delay relative to one another. This method can allow for a better approximation of the parabolic delay profile by including multiple linear delay segments. The Sparse Apodization method can reduce the number of active elements compared to the other methods by turning elements on and off in order to make the array behave similarly to a 1.5D array on transmit. One example of this sparse apodization method is shown in FIG. 23B. Note that in this approach the output pressure can be reduced compared to the full aperture. Examples of different steering angles of the transducer are shown in FIG. 44.

The results in Table 1 show the −3 dB and −10 dB beamwidths of the elevation beamplots with 0° steering in azimuth. The results show that the linear delay method is better than using no elevation focusing and can be similar to the perfect 2D focusing method. The Piecewise Linear delay method achieves even better beamwidth performance than the linear method as expected. The sparse apodization method is better than no elevation focusing in terms of achievable beamwidth but is not as good as the linear methods. The reason for the sparse apodization method underperforming is most likely due to the fact that the pitch along the “rows” of the sparse array is reduced compared to the other methods. The elevation beamplot results in FIG. 23A show that the linear and piecewise linear delay beamplots are similar to the 2D focused beamplot down to −15 dB. The sparse apodization method has an asymmetric beamplot due to the lateral offsets of the rows and this method also exhibits the largest sidelobes of all methods investigated. The methods also show stability when laterally steering off-axis (right panel of FIG. 23A). These results suggest that the aforementioned electronic elevation delays methods are suitable alternatives for phased array and linear array imaging in low-cost, battery-operated ultrasound systems.

TABLE 1

| | −3 dB | −10 dB |
|---|---|---|
| Focusing Method | Beamwdith (mm) | Beamwidth (mm) |
| No Elevation Focusing | 6.08 | 15.98 |
| Perfect 2D Focusing | 5.35 | 9.23 |
| Linear Delays | 5.38 | 9.25 |
| Piecewise Linear | 5.35 | 9.25 |
| Sparse Apodization | 5.50 | 9.65 |

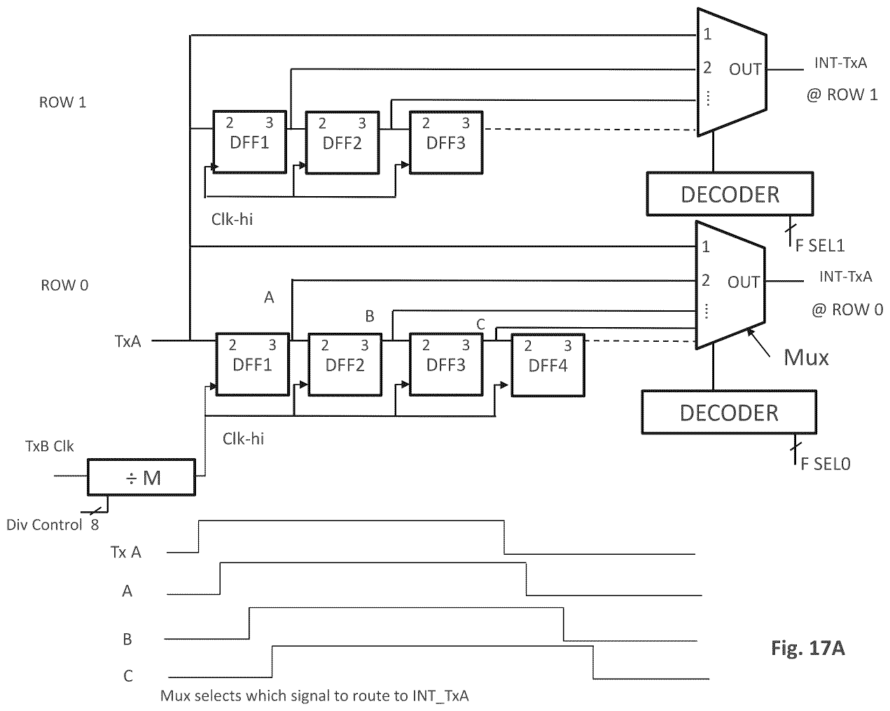

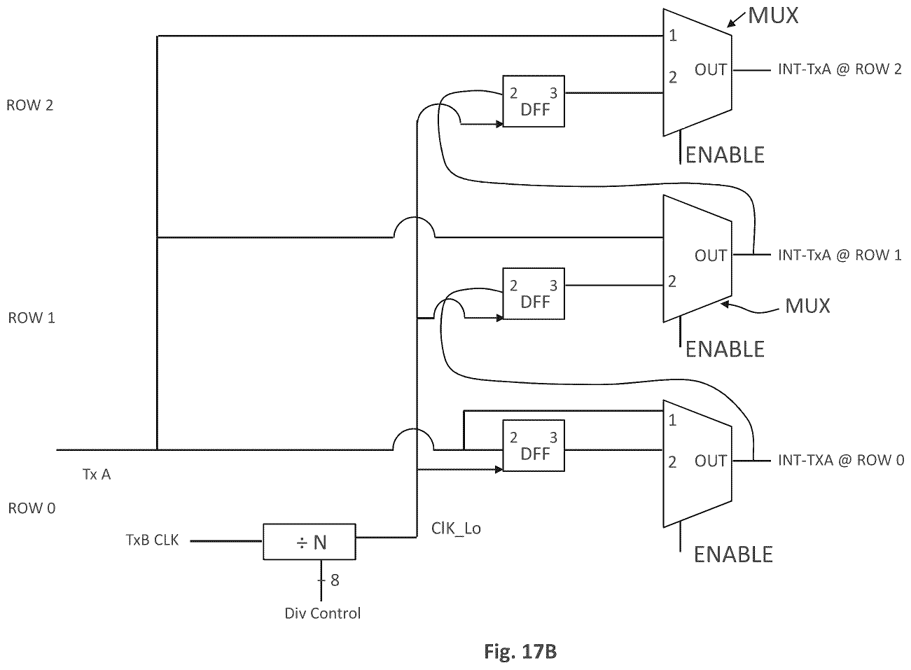

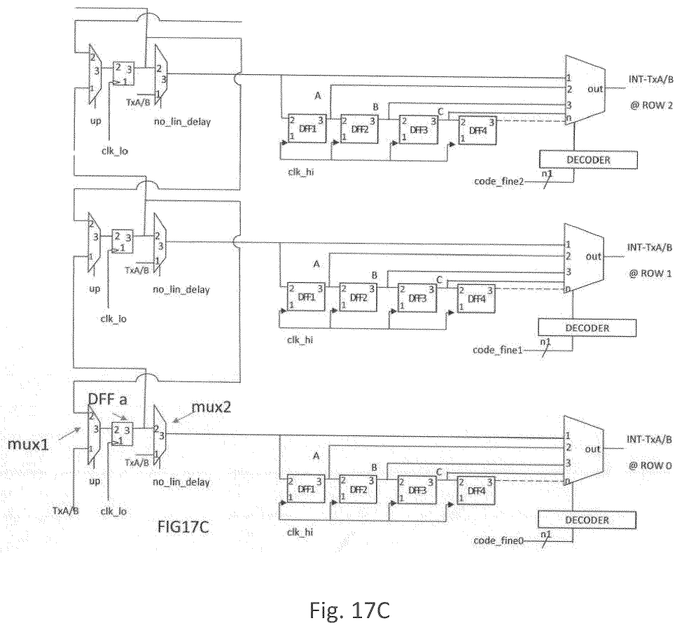

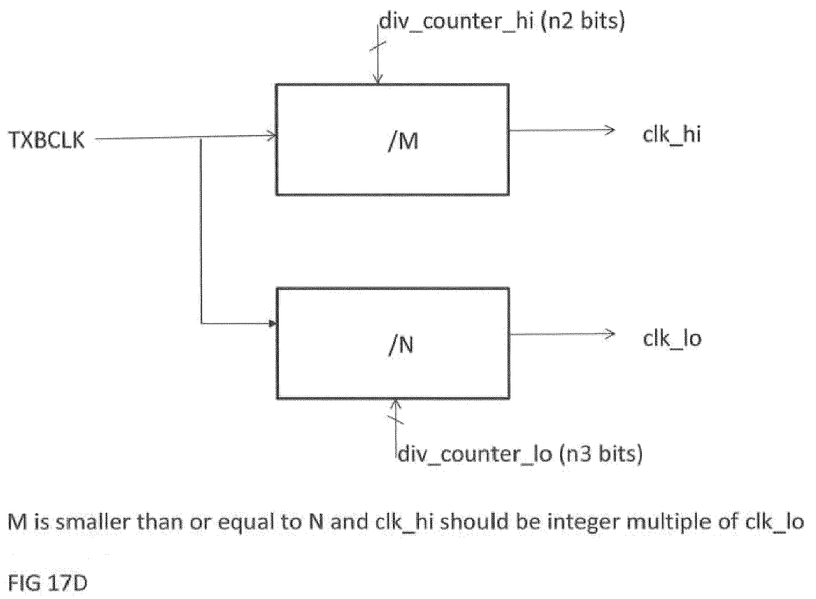

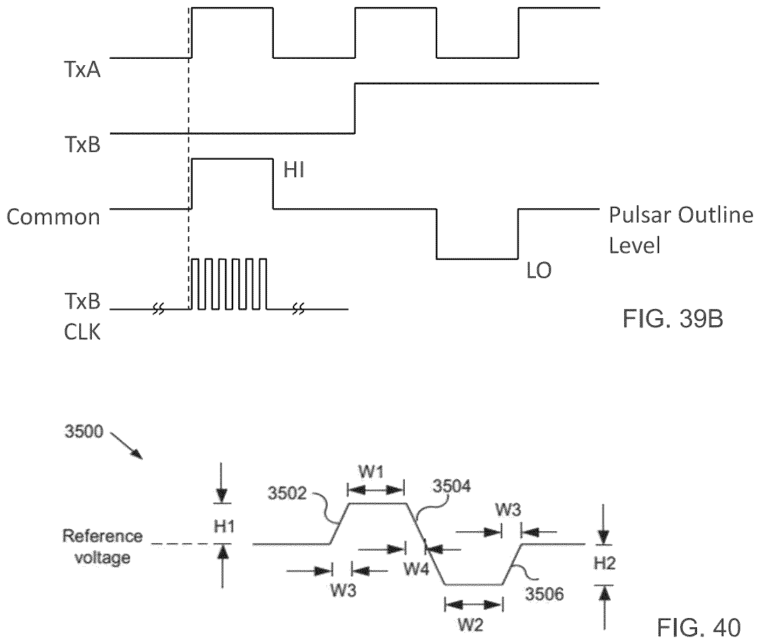

Table 1 shows elevation focus impact using various delay profiles or no focusing. These results quantify the results of the 0° azimuthal steering beamplots of FIG. 23A. In some embodiments, each element on a column has a dedicated transmit driver. In some embodiments, each element drive includes a digital delay circuit driven by a clock, e.g., TxB Clk. The delay circuit in one embodiment comprises multiple flip flops as shown in FIG. 17A. The flip flops (e.g., DFF1, DFF2, DFF3, DFF4, etc.) have a digital input starting from the bottom of the column, e.g., Row 0. In some embodiments, TxA is a digital bit generated from a Transmit beamformer. The transmit beamformer may comprise circuitry that provides multiple digital bits per channel. As shown in FIG. 17A, 2 bits per channel are used. TxA can be one digital bit. TxB can be another bit. Circuitry identical to that attached to TxA, e.g., as in FIG. 17A, can be used for TxB or any additional bits. These 2 bits can be encoded to determine the voltage drive levels for the transmit driver as shown in FIG. 39B. Here TxA and TXb are digital signals that can be decoded to determine output levels of the Tx driver. For example, if TxA, TxB are both 0 or the output level is Common or sometimes, a signal ground; if TxA=1, TxB=0, the output is HI. This can be a positive voltage of 5V or 10V or some other value as needed. When TxA=0,TxB=1, the output goes LO or −5V 05-10V for example when Common is 0V. The TxA and TxB can be created in a Tx beamformer using a high-speed clock called TxB CLK. This can be in a preferred example, a 200 MHz clock. Delayed output signals from the Tx pulsar output can be used to steer or focus ultrasound beams as shown in FIG. 18A. Here, a line imager is assumed with all elements on a line sharing same delay. Each line element can have 2 bits (e.g., TxA, TxB) sent by the Tx beamformer. The bits for the next lines are different and can be delayed depending on need to steer or focus a beam. These delays applied by the Tx beamformer can be along the azimuth axis and can steer or focus a beam in the axial direction. Delays can, however, also be needed along the elevation direction to steer or focus a beam in the elevation plane. This may require separated delays for elements on a column. FIG. 17A shows an exemplary embodiment. The TxA, TxB bits arrive from the Tx beamformer at a column. Flipflops DFF1-DFFN, where N is 1 to 16 or 32 or as large as needed, are located on every row. DFF1's input pin 1 can connect to TXA or TxB. Pin 1 of flip flops can be connected to clock named clk_hi, which is generated by a digital divider with TxB clock as its input. The division is by M, where a digital input bus labeled Div Control, shown here as an 8 bit bus as a non-limiting example, can be used to determine the value of M. Flip flops DFF1-DFFN, created delays of TxA/TxB input signals are as shown in FIG. 17A with A,B,C being delayed versions of TxA, TxB. The outputs of these can be connected to a MUX which selects one of these inputs as its output, where the selection can be done using a DECODER controlled by SEL0, SEL1, and so on, where these can include F bits. For example, for row 0, if F bits are all 0, then the input on the port labeled 1 is selected to be the output of the MUX. In case, TxA is selected to be the output. If, value of F was a binary 1, the port labeled 1 would be selected and A would be connected to the output of the MUX. These digital outputs, 2 in this case per element, can then be decoded as shown in FIG. 22 and used to drive the pulsar output. This circuit can provide fine delays with respect to incoming delays on TxA, Tx B bits for elements on a column. Further, these delays can be unique for the elements on the column. FIG. 17B shows an exemplary embodiment where coarse delays can be also added to elements on a column. Here another divider, which divides by N, can be included. The input clk TxB, where M is smaller or equal to N and are integers can be included. The output of this divider, clk_lo may be connected to the clk input of the DFF shown in FIG. 17B. Here, TxA or the output of the DFF (which is a delayed version of TxA) can be connected to a MUX and if a non-delayed version is chosen, that is applied to row 0 element. This can then be connected to pin 2 of DFF on row 1. If row 1 element needs a delay, the delayed version (pin 3 output of DFF) can be selected by MUX on row1. This can be repeated for the next element. Here a delay to all elements on the column except for element on row 0 can be added. This linear delay applied to elements up the column may help steer a beam. Circuits in FIGS. 17A and 17B can also be combined to impart a fine delay and a coarse delay to all elements on a column. For example, this can be done by adding circuitry to INT_TXA@Row0 and similar nodes on other rows, where the fine delay circuitry from FIG. 17A can be inserted to add fine delays to these outputs already delayed by a coarse delay generator. FIG. 17C shows a preferred embodiment for implementing a coarse and fine delay for each element on a column. TxA or TxB bits are shown as TxA/B connects to pin 1 of mux 1. If this input is selected by control indicated by UP, then TxA/B appears on output of mux 1. This signal can then be delayed by DFF a using clk_lo. The output of the flipflop may then be made available to mux 2 and if this input is selected by mux2 (using no_lin_delay control), then output of mux 2 connects to DFF1-N similar to that in FIG. 17A. This circuitry may provide fine delay. Following the output of DFF a, going up to a mux, similar to mux 1, but for the next row. This signal can be then delayed by DFF connected to it. The same process can be repeated going vertically to other rows. This can delay signals linearly going up the elements on a column, e.g., from row 0 to other rows. On each row, DFF1-N may add fine delay as desired to all elements on a column. A second input of mux 1 and similar mux's for all rows can be used to delay signals linearly starting with least delay on the top and largest delay at the bottom (row 0).TxA/B in this may also connect to pin2 of mux 1's clone on last row. This way, using the UP control on MUX1 (and its equivalents on other rows) the delay can increase from the bottom to the top or vice versa.

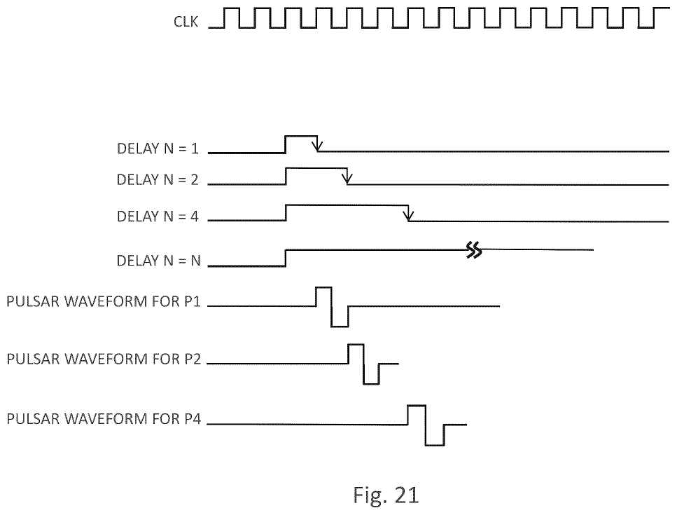

FIG. 21 shows pulsar waveforms, i.e., output of the transmit driver after delay and decoding for elevation focus is complete where P1 represents the transmit driver output for element 1 with 1 delay unit, P2 is for 2 delay units applied to element 2 and P4 is output of element 4 transmit driver with 4 delays. In this case, only coarse delay up the column is shown and no fine delay is shown in this diagram.

FIG. 18B shows relative delay for elements on a column. In some embodiments, the amount of delay determines the focal length. In some embodiments, the starting delay for all columns can be different, set by needs to focus along the azimuth axis. The delay along the elevation axis can be arbitrary. For example, delay can linearly increase from the bottom row going to the top row of the transducer. In this case, the beam can be steered in the elevation direction. If the delay is symmetric around the central element, the focus is in the elevation plane. Other various delay profiles are also possible and can allow focus and steering of the elevation slice.

FIG. 19 shows non-limiting exemplary waveforms of transmit drive pulses applied to the piezoelectric elements along a column of the transducer. In this embodiment, the transducer has 24 piezoelectric elements on a column. P0 is the piezoelectric element on a certain column (e.g., column 1, 2, 3, 4, 5, 6, 7, 8, 9, 10, 11, etc.) on row 0, P1 is the piezoelectric element on the same column as P0 but on row 1, P11 is on the same column but on row 11, P22 on row 22 and P23 on row 23. In this embodiment, one pulse at a certain frequency is applied to element P0. The same pulse is applied to element P1, but delayed by t01 with respect to P0. Similarly, the same pulse arrives at P11 with delay t011 which is longer than delay t01. In this embodiment, delays have symmetry around the central element P11. This means pulse timing at P23 and P0 are substantially identical, pulse timing at P1, P22 are substantially identical, and so forth as indicated in FIG. 19. In some embodiments, the pulse (width, magnitude, shape, and/or frequency) herein is the same for all the elements of the same column. In some embodiments, the pulse relative delay and frequency herein is the same for all the elements on 2 rows for a column, or initial delay on the 1^{st} element on a column can be different from a similar element on a different column. In some embodiments, the pulse herein has various shapes, and the waveform may have multiple pulses. Non-limiting exemplary shapes of the pulse include one or more of a rectangular pulse, a Gaussian, and a sinusoidal pulse. In some embodiments, the delays, e.g., t01, t02, t03, . . . , t011 are programmed and controlled electronically for all elements on all selected columns.

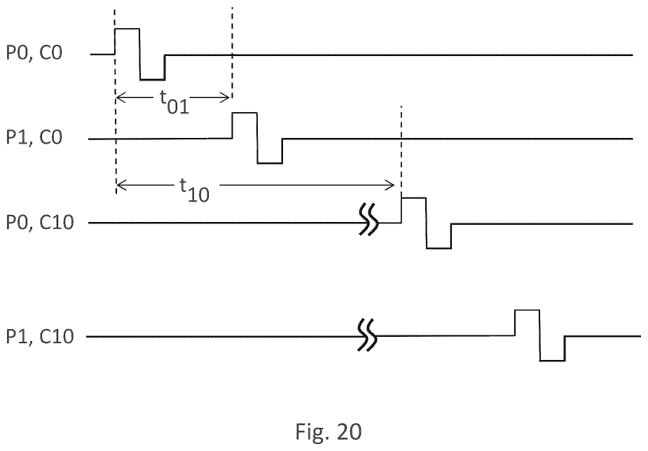

FIG. 20 shows delay relationships between columns. In this particular embodiment, the delays are determined by transmit beamformer channel delays. For example, t10 is the delay between element 0 on column 0 and element 0 on column 10. These delays are programmed in the transmit beamformer and are electrically adjustable to help focus a beam in the azimuth plane as shown in plane xa-za in FIG. 12A. In some embodiments, delays between elements on a column are separately programmed to focus a beam or tilt a beam in the elevation plane, as shown in plane ya-za in FIG. 12A. t01 is an exemplary delay between elements on a same column (e.g., elements 0 and 1 on column 0 and elements 0 and 1 on column 10). In some embodiments, delays of elements on a column are with respect to a starting delay determined by the transmit beamformer for that channel. In some embodiments, the starting delay may be predetermined by the transmit beamformer or adjustable by the transmit beamformer.

Referring to FIG. 22, in a particular embodiment, an example of the pulsar functionality is shown. In this embodiment, two digital inputs, i.e., IN1 (TxA in FIGS. 17A-17D for example), IN2 (TxB in FIGS. 17A-17D for example), control the voltage output level of the pulsar. Based on logic levels of these two inputs, a three-level output result can be generated, where HVP0 is the positive high voltage, HVM0 is the negative low voltage, and XDCR is an effective ground level or 0V. In this embodiment, five cycles of an identical pulse shape are generated as the output result. In some embodiments, by changing the IN1, IN2 pattern and/or frequency of the pattern, the pattern, frequency, and/or number of pulses of the output result can be altered. In some embodiments, the logic levels or logical codings herein may include digital logic operations of one or more inputs. In some embodiments, the logic operations include using one or more logic operators on one or more inputs selected from: AND, NOT, OR, NAND, XOR, NOR, XNOR, or any other logic operations.

In some embodiments, a cascaded series/chain of flip-flops gate the transmit clock arriving at one or more column from the transmit driver for that column with a pre-determined or pre-programmed delay that is appropriate. In some embodiments, this delay is then propagated in the column by a different clock whose frequency is programmable, but synchronized to the transmit clock that generating the delay for drivers for the various column drivers. In some embodiments, the flip-flop chain generating the delay(s) stop at the central element of the column, where the delay profile is symmetrical around the center as in FIG. 19. The delays generated by the flip-flops can be routed to the proper locations in one or more column, so row 0 element has same delay as element on the last row, the element on 2^{nd} row has similar delay as element 2^{nd} from last on top side and so on.

In embodiments, elevation focus is achieved using various delay profiles. Using a linear delay profile in the elevation direction such that delay monotonically increases or decreases from bottom to top of column may steer the beam in the elevation direction. On top of that, some additional curvature to the beam, where curvature is zero at ends of columns, may allow focus in addition to beam steering. Linear approximations of theoretical delays can be sufficiently accurate to provide steering and focus and allow economic implementations described in embodiments herein.

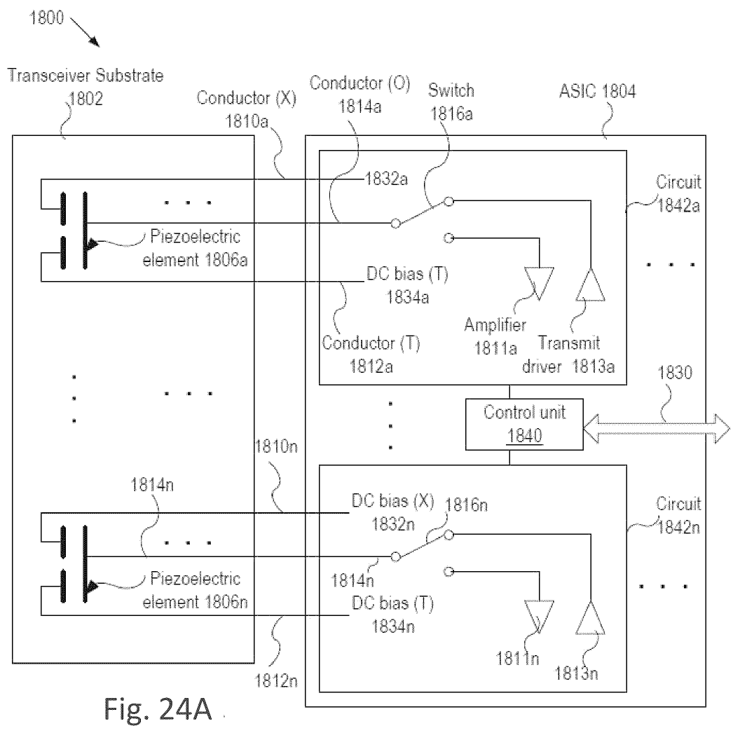

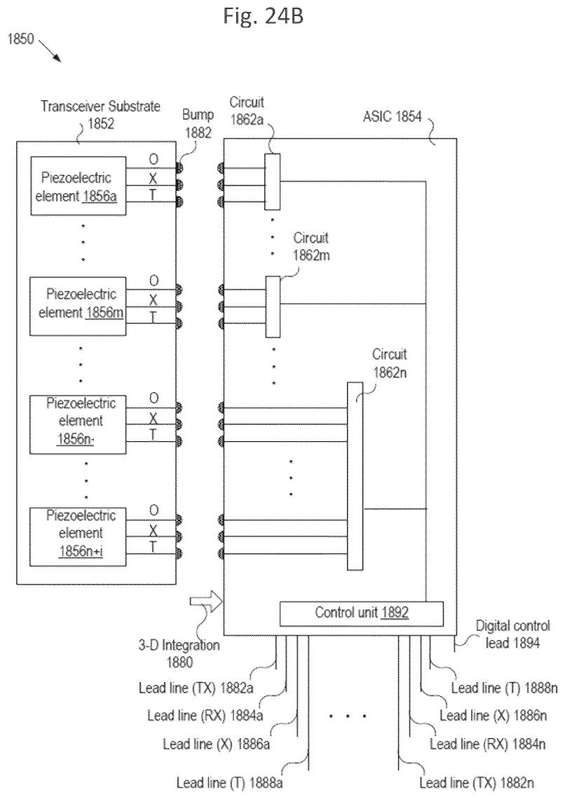

FIG. 24A shows an ultrasound imaging system 1800 herein. As depicted, the imaging system may include: a transceiver substrate 1802; and an ASIC chip 1804 electrically coupled to the transceiver substrate. In embodiments, the transceiver substrate 1802 may include one or more piezoelectric elements 1806, where each of the piezoelectric elements may be disposed on one or more membranes. In embodiments, more than one piezoelectric element may be disposed on one membrane.

In embodiments, each of the piezoelectric elements 1806a-1806n may have two or more electrodes and these electrodes may be connected to drive/receive electronics housed in the ASIC chip 1804. In embodiments, each piezoelectric element (e.g., 1806a) may include a top conductor that is electrically connected to a conductor (O) (e.g., 1814a) and two bottom electrodes that are electrically connected to conductors (X,T) (e.g., 1810a and 1812a). In embodiments, the conductor 1810a may be electrically coupled to a DC bias (X) 1832a or the ground, and the conductor (T) 1812a may be coupled to a DC bias (T) 1834a or the ground.

In embodiments, the ASIC chip 1804 may include one or more circuits 1842a-1842n that are each electrically coupled to one or more piezoelectric elements 1806a-1806n; and one control unit 1840 for controlling the circuits 1842a-1842n. In embodiments, each circuit (e.g., 1842a) may include a transmit driver (1813a), a receiver amplifier (e.g., 1811a), a switch (e.g., 1816a) having one terminal electrically coupled to the conductor (O) (1814a) and another terminal that toggles between the two conductors coupled to the transmit driver 1813a and amplifier 1811a. During a transmit (Tx) mode/process, the switch 1816a may connect the transmit driver 1813a to the piezoelectric element 1806a so that a signal is transmitted to the top electrode of the piezoelectric element 1806a. During a receive (Rx) mode/process, the switch 1816a may connect the amplifier 1811a to the piezoelectric element 1806a so that a signal is transmitted from the top electrode of the piezoelectric element 1806a to the amplifier 1811a.

In some embodiments, the transmit driver 1813a may include various electrical components. However, for brevity, the transmit driver 1813a is represented by one driver. But, it should be apparent to those of ordinary skill in the art that the transmit driver may include a more complex driver with many functions. Electrical components for processing the received signals may be connected to the amplifier 1811a, even though only one amplifier 1811a is shown in FIG. 24A. In embodiments, the amplifier 1811a may be a low noise amplifier (LNA). In embodiments, the circuit 1842n may have the same or similar structure as the circuit 1842a.

In embodiments, all of the DC biases (X) 1832a-1832n may be connected to the same DC bias or the ground, i.e., all of the conductors (X) 1810a-1810n may be connected to a single DC bias or the ground. Similarly, all of the DC biases (X) 1834a-1834n may be connected to the same DC bias or a different DC bias, i.e., all of the conductors (T) 1812a-1812n may be connected to a single DC bias or the ground.

In embodiments, the conductors (X, T and O) 1810, 1812, and 1814 may be connected to the ASIC chip 1804 using an interconnect technology—for instance, copper pillar interconnects or bumps (such as 1882 in FIG. 24B), as indicated by an arrow 1880. In embodiments, the circuitry components in the ASIC chip 1804 may communicate outside the ASIC chip 1804 using an interconnect 1830. In embodiments, the interconnect 1830 may be implemented using bonding wires from pads on the ASIC chip 1804 to another pad outside the ASIC chip. In embodiments, other types of interconnects, such as bump pads or redistribution bumps on the ASIC chip 1804 may be used in addition to wire bonded pads.

In embodiments, the LNAs 1811 included in the circuits 1842 may be implemented outside the ASIC chip 1804, such as part of a receive analog front end (AFE). In embodiments, a LNA may reside in the ASIC chip 1804 and another LNA and programmable gain amplifier (PGA) may reside in the AFE. The gain of each LNA 1811 may be programmed in real time, allowing the LNA to be part of a time gain compensation function (TGC) needed for the imager.

In embodiments, the LNAs 1811 may be built using low voltage transistor technologies and, as such, may be damaged if they are exposed to high transmit voltages that the conventional transducers need. Therefore, in the conventional systems, a high voltage transmit/receive switch is used to separate the high transmit voltages from the low voltage receive circuitry. Such a switch may be large and expensive, use High Voltage (HV) processes, and degrade the signal sent to LNA. In contrast, in embodiments, low voltages may be used, and as such, the high voltage components of the conventional system may not be needed any more. Also, in embodiments, by eliminating the conventional HV switch, the performance degradation caused by the conventional HV switch may be avoided.

In embodiments, the piezoelectric elements 1806 may be connected to the LNAs 1811 during the receive mode by the switches 1816. The LNAs 1811 may convert the electrical charge in the piezoelectric elements 1806 generated by the reflected pressure waves exerting pressure on the piezoelectric elements, to an amplified voltage signal with low noise. The signal to noise ratio of the received signal may be among the key factors that determine the quality of the image being reconstructed. It is thus desirable to reduce inherent noise in the LNA itself. In embodiments, the noise may be reduced by increasing the transconductance of the input stage of the LNAs 1811, such as using more current in the input stage. The increase in current may cause the increase in power dissipation and heat. In embodiments, pMUTs 1806 may be operated with low voltages and be in close proximity to the ASIC chip 1804, and as such, the power saved by the low voltage pMUTs 1806 may be utilized to lower noise in the LNAs 1811 for a given total temperature rise acceptable, compared to conventional transducers operated with high voltages. FIG. 24B shows a schematic diagram of an imaging assembly 1850 according to embodiments of the present disclosure. In embodiments, the transceiver substrate 1852 and ASIC chip 1854 may be similar to the transceiver substrate 1802 and ASIC chip 1804, respectively. In the conventional systems, the electronics for driving piezoelectric transducers is typically located far away from the piezoelectric transducers and are connected to the piezoelectric transducers using a coax cable. In general, the coax cable increases parasitic loading, such as additional capacitance, on the electronics, and the additional capacitance causes loss in critical performance parameters, such as increase in noise and loss of signal power. In contrast, as depicted in FIG. 24B, the transmit driver or drivers (or equivalently circuits) 1862a-1862n may be connected directly to piezoelectric elements (or equivalently pixels) 1856a-1856n+i using a low impedance three-dimensional (3D) interconnect mechanism (as indicated by an arrow 1880), such as Cu pillars or solder bumps 1882, or wafer bonding or similar approaches or a combination of such techniques. In embodiments, upon integrating the transceiver substrate 1852 to the ASIC chip 1854, the circuits 1862 may be located less than 100 μm vertically (or so) away from the piezoelectric elements 1856. In embodiments, any conventional device for impedance matching between driver circuits 1862 and piezoelectric elements 1856 may not be required, further simplifying design and increasing power efficiency of the imaging assembly 1800. Impedance of the circuits 1862 may be designed to match the requirement of the piezoelectric elements 1856.

In embodiments, in FIG. 24A, each of the piezoelectric elements 1806a-1806n may be electrically connected to a corresponding one of the circuits 1842a-1842n located in the ASIC chip 1804. Signals at the inputs of transmit drivers 1813a-1813n can be generated using circuits for example, shown in FIGS. 17A-D but not explicitly shown in FIGS. 24A and 24B. In embodiments, this arrangement may allow the imager to generate three-dimensional images. Similarly, in FIG. 24B, each of the piezoelectric elements 1856a-1856m may have three leads represented by X, T, and O. The leads from each of the piezoelectric elements may be electrically connected to a corresponding one of the circuits 1862a-1862m located in the ASIC chip 1854 by the interconnect means 1882. In addition, in embodiments, a line of piezoelectric elements, such as 1856n-1856n+i may be electrically coupled to one common circuit 1862n. In embodiments, the transmit driver circuit 1862n may be implemented with one transmit driver. In alternative embodiments, the transmit driver circuit 1862n may be implemented with multilevel drivers to facilitate various imaging modes.

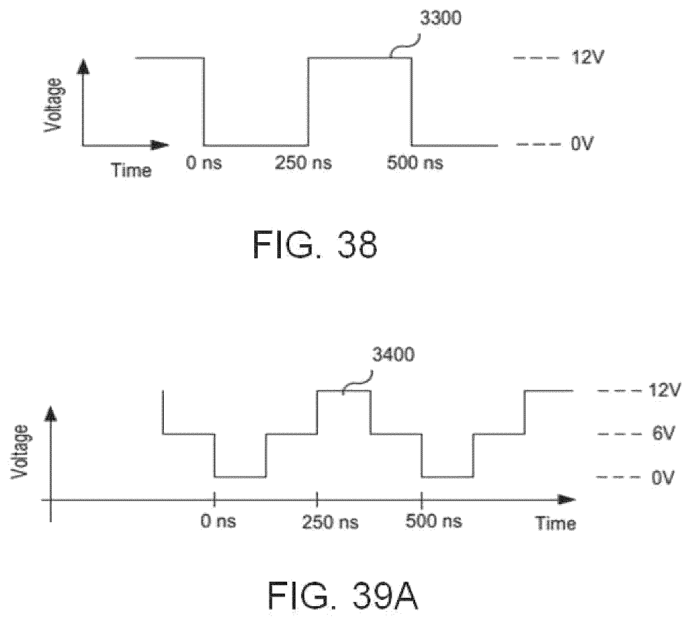

It should be apparent to those of ordinary skill in the art that the ASIC chip 1854 may have any suitable number of circuits that are similar to the circuit 1862n. In embodiments, the control unit 1892 may have capability to configure the piezoelectric elements, either horizontally or vertically in a two-dimensional pixel array, configure their length and put them into transmit or receive or poling mode or idle mode. In embodiments, the transmit driver circuit 1813 may be implemented with multilevel drive as shown in FIGS. 22 and 39, where the transmit driver output may have more than 2 output levels. FIG. 39A shows an embodiment where the output level may be 0V or 6V or 12V. It is understood that that these voltages can be different, for example they can be −5V, 0V and +5V. The transmit driver can also be a 2-level driver with drive signal as shown in FIG. 38.

In embodiments, lead lines 1882a-1882n may be signal conductors that are used to apply pulses to the electrodes (O) of the piezoelectric elements 1856. Similarly, the lead lines 1884a-1884n, 1886a-1886n, and 1888a-1888n may be used to communicate signals with the piezoelectric elements 1856a-1856n+i. It is noted that other suitable number of lead lines may be used to communicate signals/data with the imaging assembly 1800.

In embodiments, each of the lead lines (X) 1886 and lead lines (T) 1888 may be connected to the ground or a DC bias terminal. In embodiments, the digital control lead 1894 may be a digital control bus and include one or more leads that are needed to control and address the various functions in the imaging assembly 1850. These leads, for example, may allow programmability of the ASIC chip 1854 using communication protocols, such as Serial Peripheral Interface (SPI) or other protocols.

In embodiments, the piezoelectric elements 1806 (or 1856) and the control electronics/circuits 1842 (or 1862) may be developed on the same semiconductor wafer. In alternative embodiments, the transceiver substrate 1802 (or 1852) and ASIC chip 1804 (or 1854) may be manufactured separately and combined to each other by a 3D interconnect technology, such as metal interconnect technology using bumps 1882. In embodiments, the interconnect technology may eliminate the low yield multiplication effect, to thereby lower the manufacturing cost and independently maximize the yield of components.

In embodiments, lead lines 1862a-1862n may be signal conductors that are used to apply pulses to the electrodes (O) of the piezoelectric elements 1806. Similarly, the lead lines 1864a-1864n, 1866a-1866n, and 1868a-1868n may be used to communicate signals with the piezoelectric elements 1806a-1806n. It is noted that other suitable number of lead lines may be used to communicate signals/data with the imaging assembly 1800.

As discussed above, the LNAs 1811 may operate in a charge sensing mode and each have a programmable gain that may be configured in real time to provide gain compensation.

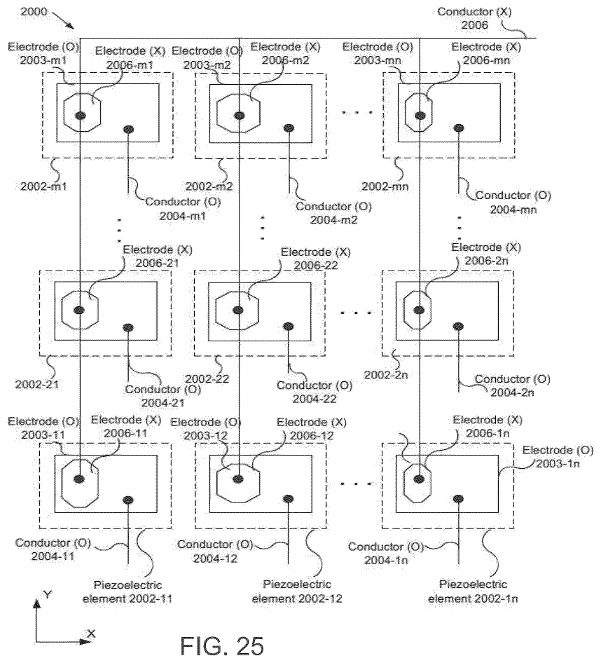

FIG. 25 shows a schematic diagram of an m×n array 2000 of piezoelectric elements 2002-11-2002-mn according to embodiments of the present disclosure. As depicted, each piezoelectric element may be a two terminal piezoelectric element (such as piezoelectric element 214 in FIG. 3A) and have an electrode (O) (e.g., 2003-11) electrically coupled to a conductor (O) (e.g., 2004-11) and an electrode (X) electrically connected to ground or a DC bias voltage via a common conductor (X) 2006. In embodiments, each signal conductor (O) may be managed independently by a circuit element). In embodiments, each conductor (O) (e.g., 2004-mn) may be electrically coupled to a transmit driver of a circuit element while all of the X electrodes (2006-11-2006-mn) of the piezoelectric element array may be connected to a common conductor (X) 2006. In embodiments, the array 2000 may be disposed on a transceiver substrate and electrically coupled to an ASIC chip by interconnection mechanism, such as m×n+1 bumps. More specifically, the m×n conductors (O) 2004-11-2004-mn may be coupled to m x n transmit drivers of ASIC chip by m×n bumps and the common conductor (X) 2006 may be coupled to the ASIC chip by one bump. In embodiments, such an exemplary arrangement as described here is used to perform 3D imaging, where each piezoelectric element, including at least one sub piezoelectric element, can provide unique information in the array. In embodiments, each piezoelectric element may have one or more membranes and vibrate in multiple modes and frequencies of the membranes. In embodiments, each piezoelectric element 2002 may be driven by pulses that have voltage profiles 3300 and 3400 in FIGS. 38 and 39.

In embodiments, the O electrodes in each column (e.g., 2003-11-2003-m1) may be electrically coupled to a common conductor. For instance, the circuit elements in the ASIC chip may be electronically controlled so that the O electrodes in each column may be electrically coupled to each other. In such a configuration, the O electrodes in each column may receive the same electrical pulse through a common transmit driver or per a multiplicity of drivers with identical electrical drive signals during the transmit mode. Similarly, the O electrodes in each column may simultaneously transmit the electrical charge to a common amplifier during the receive mode. Stated differently, the piezoelectric element in each column may be operated as a line unit (or equivalently line element).

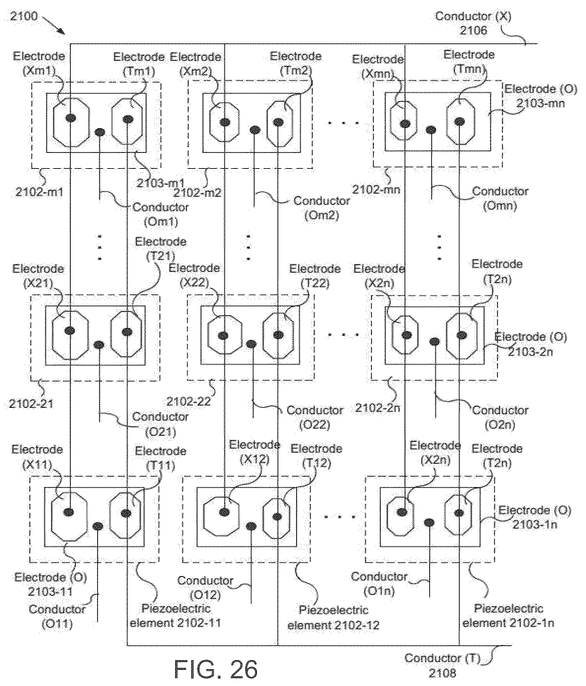

FIG. 26 illustrates a schematic diagram of an n×n array 2100 of piezoelectric elements 2102-11-2012-mn according to embodiments of the present disclosure. As depicted, each piezoelectric element may be a three terminal piezoelectric element and include the electrodes O, X, and T. In embodiments, the X electrodes (e.g., X11, X21, . . . , Xm1) may be connected column wise in a serial manner and all of the X electrodes (X11-Xmn) may be electrically coupled to a common conductor (X) 2106. The T electrodes (e.g., T11, T21, . . . , Tm1) may be connected column wise in a serial manner and all of the T electrodes (T11-Tmn) may be electrically coupled to a common conductor (T) 2108. A column of elements such as 2102-11, 2102-21 through 2102-m1 when connected together as described in embodiments make up a line element or a column. In embodiments, each of the O electrodes 2103-11-2103-mn may be electrically coupled to a transmit driver of a corresponding circuit element in an ASIC chip via one of the conductors O11-Omn. In embodiments, the array 2100 may be disposed on a transceiver substrate and electrically coupled to an ASIC chip by interconnection mechanism, such as m×n+2 bumps. In the ASIC, transmit drivers connected to O electrodes can receive decoded pulses to create a multilevel output as shown in FIG. 22. These pulses can be delayed for elements along a column (as shown in FIGS. 17A-17D). Also, delays can be created along columns (as illustrated for example in FIG. 19).

In embodiments, the O electrodes in each column (e.g., 2103-11-2103-m1) may be electrically coupled to a common conductor. In such a configuration, the O electrodes in each column may receive the same electrical pulse through a common transmit driver during the transmit mode. Similarly, the O electrodes in each column may simultaneously transmit the electrical charge to a common amplifier during the receive mode. Stated differently, the piezoelectric element in each column is operated as a line unit. In embodiments each of the O electrodes in a column may be connected to a dedicated transmit driver, where the input signal of the transmit drivers for all elements in a column are identical, thus creating a substantially identical transmit drive output to appear on all piezoelectric elements during a transmit operation. Such a line element is electronically controlled on a per element basis, since each element has its own transmit driver. This has advantages in driving large capacitive line elements, where each element has smaller capacitance and delays in timing can be minimized for elements on a column. In embodiments, in a receive mode, charge from all elements in a column can be sensed by connecting it to a LNA, as is done by 2D imaging. For 3D imaging, charge for each element is sensed by connecting the O electrodes of each element to a LNA during a receive mode operation.

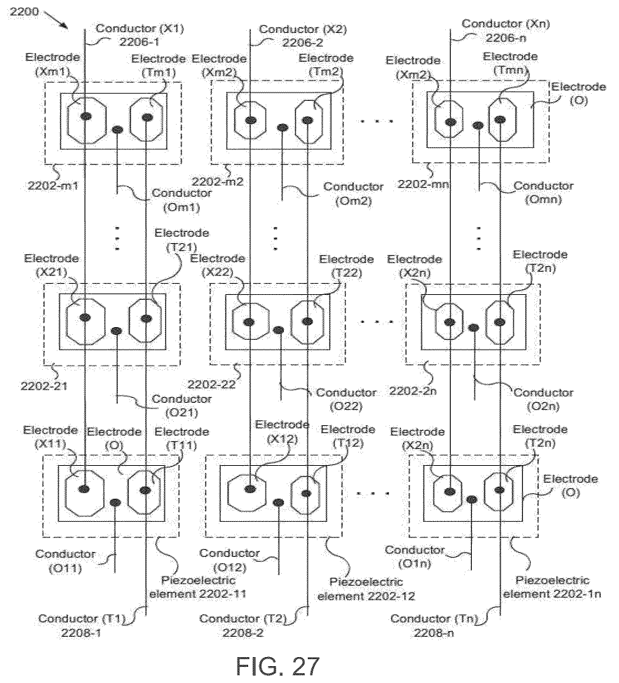

FIG. 27 illustrates a schematic diagram of an m×n array 2200 of piezoelectric elements 2202-11-2202-mn according to embodiments of the present disclosure. As depicted, the array 2200 may be similar to the array 2100, with the differences that the X electrodes (e.g., X12-Xm2) in a column may be connected to a common conductor (e.g., 2206-1) and the T electrodes (e.g., T12-Tm2) in a column may be connected to a common conductor (e.g., 2208-1). As such, the X electrodes (or T electrodes) in the same column may have the same voltage potential during operation. In embodiments, each of the O electrodes may be electrically coupled to a transmit driver of a corresponding circuit element in an ASIC chip via one of the conductors O11-Omn. In embodiments, the array 2200 may be disposed on a transceiver substrate and electrically coupled to an ASIC chip by interconnection mechanism, such as m×n+2n bumps.

Compared to array 2100, the array 2200 may use more bumps for connecting the T and X electrodes to the ASIC chip. In general, an increase in the number of connections for T and X between the ASIC chip and the piezoelectric array may reduce impedance in the X and T conductors when connected in parallel to the ground or DC bias sources and reduce the crosstalk. Crosstalk refers to the coupling of signals from an imaging element to another one, and may create interference and reduce the image quality. Spurious electrical coupling may be created when any voltage drop due to current flowing in X and T lines appear across a piezoelectric element that ideally should not be exposed to that voltage. In embodiments, when the piezoelectric element is not transmitting or receiving under electronic control, the X, T, and O electrodes may be locally shorted. Alternatively, the idle electrodes have the O electrodes grounded, leaving the X electrodes connected to other X electrodes in the array and the T electrodes connected to other T electrodes in the array.

FIG. 28 illustrates a schematic diagram of an m×n array 2300 of piezoelectric elements 2301-11-2302-mn according to embodiments of the present disclosure. As depicted, the array 2300 may be similar to the array 2100, with the difference that each piezoelectric element may be a five terminal piezoelectric element, i.e., each piezoelectric element may include one bottom electrode (O) and four top electrodes (two X electrodes and two T electrodes). In embodiments, two X electrodes of each piezoelectric element may be connected column wise in a serial manner and all of the 2m×n X electrodes may be electrically coupled to a common conductor (X) 2306. Similarly, the two T electrodes of each piezoelectric element may be connected column wise in a serial manner and all of the 2m×n T electrodes may be electrically coupled to a common conductor (T) 2308. In embodiments, each of the O electrodes may be electrically coupled to a transmit driver of a corresponding circuit element in an ASIC chip via one of the conductors O11-Omn. The ASIC may contain transmit drivers that connect to the O nodes and inputs to the transmit drivers may be delayed using techniques and circuits discussed herein for example in FIGS. 17A-D. In embodiments, the array 2300 may be disposed on a transceiver substrate and electrically coupled to an ASIC chip by interconnection mechanism, such as m×n+2 bumps.

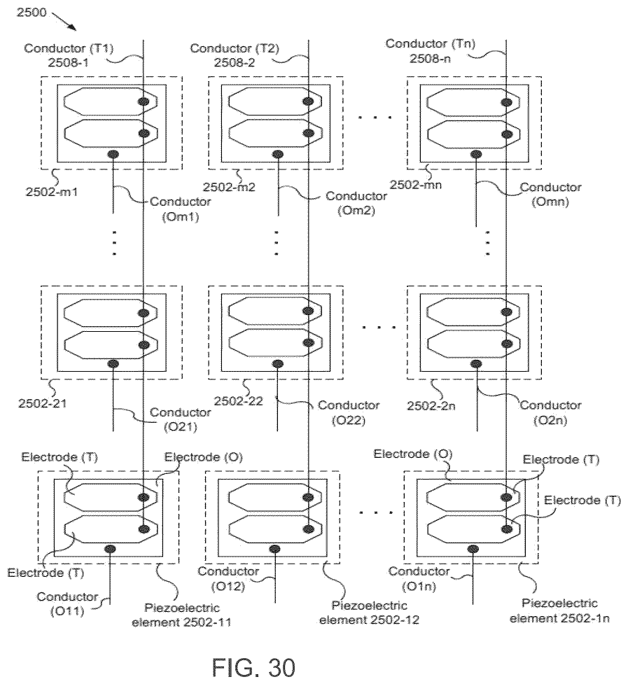

Referring to FIG. 28, in this exemplary embodiment, two subelements are connected to an X electrode and 2 other subelements are connected to the T electrode. X and T electrodes can be bias electrodes which are connected to DC voltage sources. The 2 elements connected to the X electrode may have different resonant frequency behavior. Together, these 2 subelements may exhibit wider bandwidth than each subelement by itself. The 2 subelements with 1 terminal connected to the T electrode may both exhibit similar resonant frequency behavior as the elements connected to the X electrode. For example, 1 subelement connected to X electrode and 1 subelement connected to T electrode may have a resonant frequency or center frequency of 2 MHz and the remaining subelements may exhibit a center frequency of 4 MHz. By combining these 2 subelements, the bandwidth of the composite elements becomes wider. The use of X and T electrodes can also allow different polarization direction in the subelement(s), increasing sensitivity of the element(s). In principle, however, a wide band element can also be designed using only X or T electrode as is shown in FIG. 30.

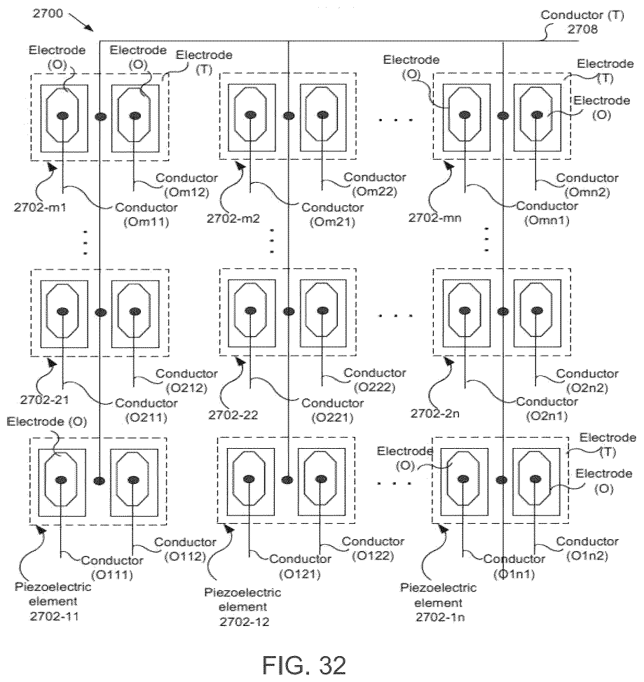

In another exemplary embodiment shown in FIG. 32, an element still uses 2 subelements, but in this case, each of the subelements may have 2 “O” terminals. Each sub element can exhibit different behavior and because each subelement has a unique controllable drive terminal, the O electrode, they can be electronically driven independently.

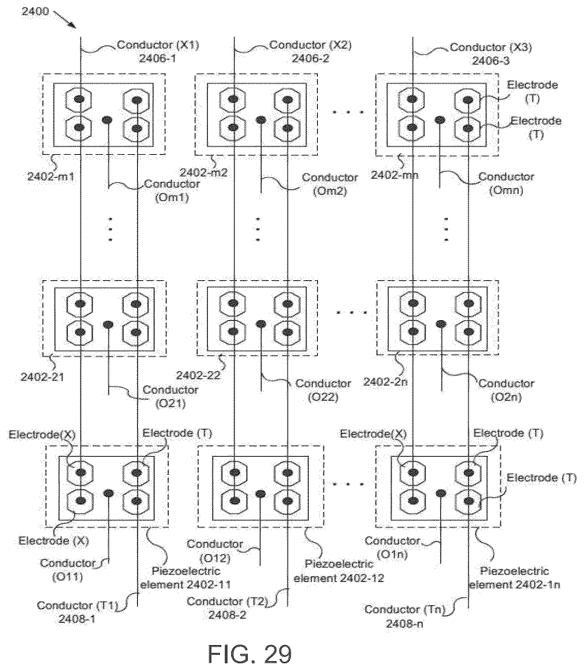

FIG. 29 illustrates a schematic of an m×n array 2400 of piezoelectric elements 2402-11-2402-mn according to embodiments of the present disclosure. As depicted, the array 2400 may be similar to the array 2200, with the differences that each piezoelectric element may be a five terminal piezoelectric element: one bottom electrode (O) and four top electrodes (two X electrodes and two T electrodes). In embodiments, the two X electrodes of each piezoelectric element may be electrically connected in a column wise direction to a conductor (e.g., 2406-1), and the two T electrodes of each piezoelectric element may be electrically connected in a column wise direction to a common conductor (e.g., 2408-1). In embodiments, each of the O electrodes may be electrically coupled to a transmit driver of a corresponding circuit element in an ASIC chip via one of the conductors O11-Omn. In embodiments, the array 2400 may be disposed on a transceiver substrate and electrically coupled to an ASIC chip by interconnection mechanism, such as m×n+2n bumps.

FIG. 30 illustrates a schematic diagram of an m×n array 2500 of piezoelectric elements 2505-11-2502-mn according to embodiments of the present disclosure. As depicted, the array 2500 may be similar to the array 2100 in that each piezoelectric element may have one bottom electrode (O) and two top electrodes (T), but have the differences that all of the two top electrodes (T) of the piezoelectric elements along a column (e.g., 2505-11-2502-m1) may be electrically connected to a common conductor (e.g., 2508-1). In embodiments, each of the O electrodes may be electrically coupled to a transmit driver of a corresponding circuit element in an ASIC chip via one of the conductors O11-Omn. In embodiments, the array 2500 may be disposed on a transceiver substrate and electrically coupled to an ASIC chip by interconnection mechanism, such as m×n+n bumps.

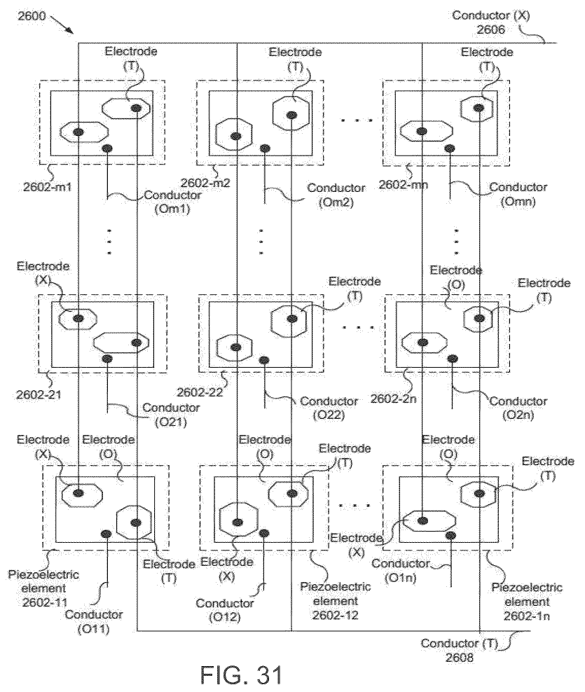

FIG. 31 illustrates a schematic of an m×n array 2600 of piezoelectric elements 2602-11-2602-mn according to embodiments of the present disclosure. As depicted, the array 2600 may have similar electrical connections to the array 2100, i.e., all of the X electrodes in the piezoelectric elements may be electrically coupled to a common conductor 2606 and all of the T electrodes in the piezoelectric elements may be electrically coupled to a common conductor 2608. The array 2600 may be different from the array 2100 in that the top electrodes (X, T) of one piezoelectric element (e.g., 2602-11) may have the same or different geometrical shapes from the top electrodes (X, T) of another piezoelectric element (e.g., 2602-21).

For the piezoelectric arrays 2000-2500, the piezoelectric elements in each piezoelectric array may be the same or different from each other. For instance, the projection areas of the two top electrodes of one piezoelectric element 2202-11 may have same or different shapes from the projection areas of the two top electrodes of another piezoelectric element 2202-n1.

FIG. 32 illustrates a schematic diagram of an m×n array 2700 of piezoelectric elements 2702-11-2702-mn according to embodiments of the present disclosure. As depicted, each piezoelectric element may include two signal electrodes (O) and one common electrode (X). In embodiments, each signal electrode (O) may be electrically coupled to a transmit driver of a corresponding circuit element of an ASIC chip. For instance, the piezoelectric element 2702-11 may include two signal conductors O111 and O112 that may be electrically coupled to two circuit elements in an ASIC chip, respectively, where each signal electrode may develop electrical charge during the receive mode. In embodiments, the array 2700 may be disposed on a transceiver substrate and electrically coupled to an ASIC chip by interconnection mechanism, such as 2m×n+1 bumps. In embodiments, all of the T electrodes in the array 2700 may be electrically coupled to the ground or a DC bias voltage via the common conductor (T) 2708.

In embodiments, the signal conductors (O) in the arrays, e.g., as in FIGS. 25-32, may be electrically coupled to a circuit element, where the circuit element may include a transistor switch that is similar to the switch 1816 in FIG. 24A, i.e., the switch may toggle between the transmit driver and an amplifier during the transmit and receive modes, respectively so that the O electrode may generate pressure waves during the transmit mode and develop electrical charge during the receive mode.

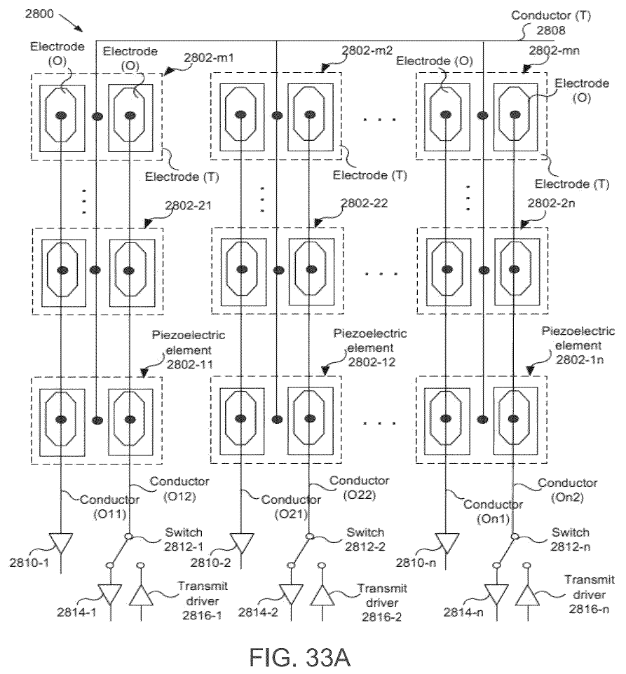

FIG. 33A shows an exemplary embodiment of an imaging system 2800 according to embodiments of the present disclosure. As depicted, the imaging system 2800 may include an array of piezoelectric elements 2802-11-2802-mn and circuit elements for controlling/communicating with the array. In embodiments, each of the piezoelectric elements 2802-11-2802-mn may include three electrodes; first and second signal (O) electrodes and a T electrode. (For the purpose of illustration, the first and second O electrodes in each piezoelectric element refer to the left and right O electrodes of each piezoelectric element in FIG. 33.) In embodiments, all of T electrodes in the array 2800 may be electrically coupled to the ground or DC bias voltage via the conductor (T) 2808. In embodiments, the first O electrodes of the piezoelectric elements in a column may be electrically coupled to a common conductor (e.g., O11) and the second O electrodes of the piezoelectric elements in the same column may be electrically coupled to another common conductor (e.g., O12). In this embodiment, elevation steering electronically may not possible since for example all right O electrodes for 2802-11 through 2802-m1 may be connected together using 012 and then connected to one Tx driver or Rx receiver 2816-1 and 2814-1. Instead of hard connecting the O nodes for each element on a column together using 012, each O node on that column can be connected to a corresponding Tx driver and then delays of signals sent to the transmit driver can be controlled such that elements on a column can have different delay, as such, electronic focus may be achieved in the elevation axis for this column. This is shown in FIG. 33B, in this embodiments, where switch 2812-11 connects to pMUT element 2802-21 and switch 2812-1m connects to O terminal of 2802-m1. The inputs of Tx drivers 2816 are connected to circuits that create desired delay between elements such as shown in FIGS. 17A-17D. A separate Tx driver, receive amplifier and/or switch MAY BE needed for every O electrode for elements where electronic focus is desired. In this example, other pMUT elements that require synthetic elevation focus are also shown with separate Transmit and receive electronics. Their presentation is shown in simplified form in FIG. 33B, but is intended to represent functional need for transmitting and receiving of signals. In this example, if elements on O11-On1 do not require electronic focus, they can be hardwired as shown. In embodiments, during the receive mode, each of the first and second signal O electrodes may develop electrical charge that may be processed by a corresponding circuit.

In embodiments, as shown in FIG. 33A, the first set of conductors O11, O21, . . . , On1 may be electrically coupled to the amplifiers 2810-1-2810-n, respectively, where the electrical charge developed in a column of the first O electrodes may be transferred to a corresponding amplifier via one of the O conductors. In embodiments, the second set of conductors O12, O22, . . . , On2 may be electrically coupled to the switches 2812-1-2812-n, respectively, assuming no electronic focus along the elevation axis is needed. Otherwise, each element on O11, On2 may have a Tx driver and Rx amp connected to it through a switch, as shown in FIG. 33B. In embodiments, each switch may be connected to a transit driver during the transmit mode/process so that a signal pulse may be transmitted to a column of second O electrodes in the piezoelectric elements. In embodiments, each switch (e.g., 2812-1) may be connected to a signal amplifier (e.g., 2814-1) during the receive mode/process so that electrical charge developed in a column of second O electrodes in the piezoelectric elements (e.g., 2801-11-2802-m1) may be transmitted to the amplifier. In embodiments, the piezoelectric elements 2802-11-2802-mn may be disposed in a transceiver substrate while the switches 2812-1-2812-n, transmit drivers 2816-1-2816-n, and amplifies 2810-1-2810-n and 2814-1-2814-n may be disposed in an ASIC chip, where the transceiver substrate may be electrically coupled to the ASIC chip by 2n+1 bumps. It is understood that while the previous explanation refers to FIG. 33A, extensions where each O electrode is connected to corresponding Tx driver and Rx amplifier through a switch can be used to achieve elevation focus electronically as shown in FIG. 33B.

In embodiments, the transmit driver (e.g., 2816-1) may send a signal to a column of piezoelectric elements (e.g., 2802-11-2802-m1) via a conductor (012) and simultaneously, an amplifier (e.g., 2810-1) may receive electrical charge signal from the same column of piezoelectric elements (e.g., 2802-11-2802-m1). In such a case, each piezoelectric element (e.g., 2802-11) in a column may receive a signal from the transmit driver (e.g., 2816-1) through one conductor (e.g., O12) and simultaneously transmit an electrical charge signal to an amplifier (e.g., 2810-1) via another conductor (e.g., O11), i.e., the imaging system 2800 may perform simultaneous transmitting and receiving modes. This simultaneous operation of transmitting and receiving modes may be very advantageous in continuous mode Doppler Imaging, where a high blood flow velocity may be imaged, compared to pulsed Doppler Imaging.

In embodiments, a line unit, which refers to a column of O electrodes electrically coupled to a common conductor, may operate as a transmit unit or a receive unit or both. For instance, electrical signals may be sequentially transmitted to the conductors O12, O22, . . . , On2 so that the line elements sequentially generate pressure waves during the transmit mode, and the reflected pressure waves may be processed and combined to generate a two dimensional image of the target organ in the receive mode. In another example, electrical drive signals may be simultaneously transmitted to the conductors O12, O22, . . . , On2 during the transmit mode and the reflected pressure waves may be processed at the same time using charge generated from conductors O11, O12 to On1 to simultaneously transmit and receive ultrasound to create a two dimensional image. Conductors O12-On2 may also be used to receive charge from the piezoelectric line elements in a receive mode of operation.

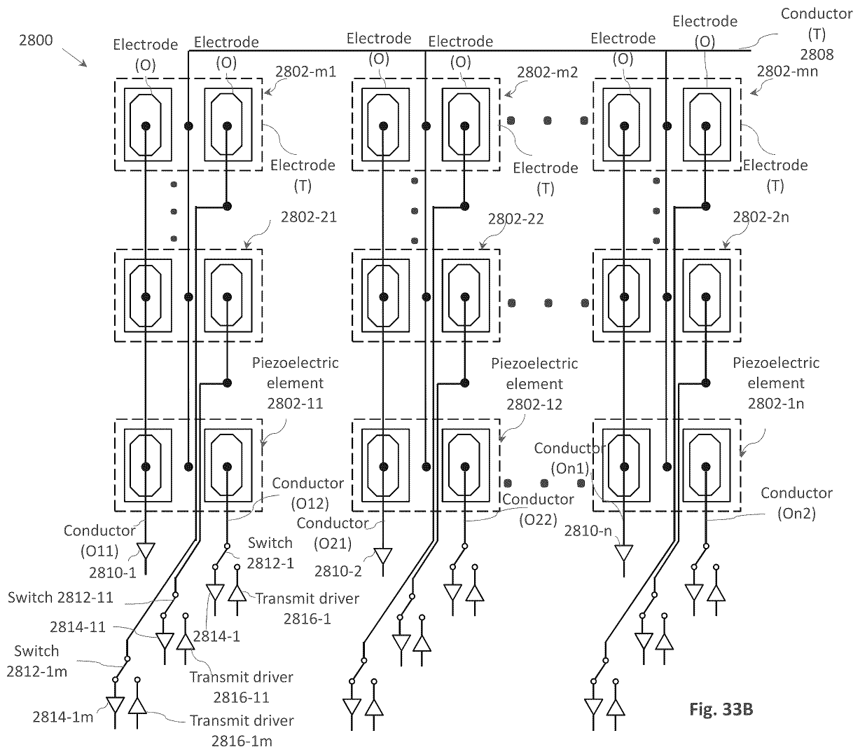

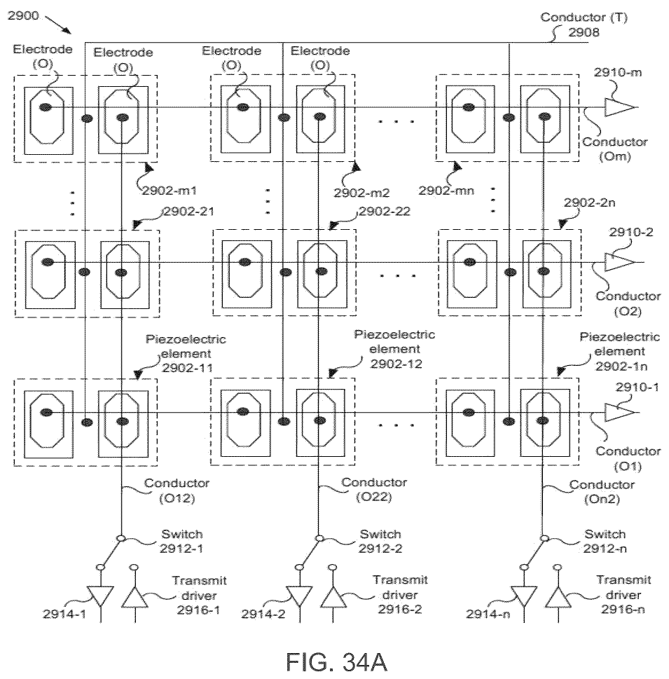

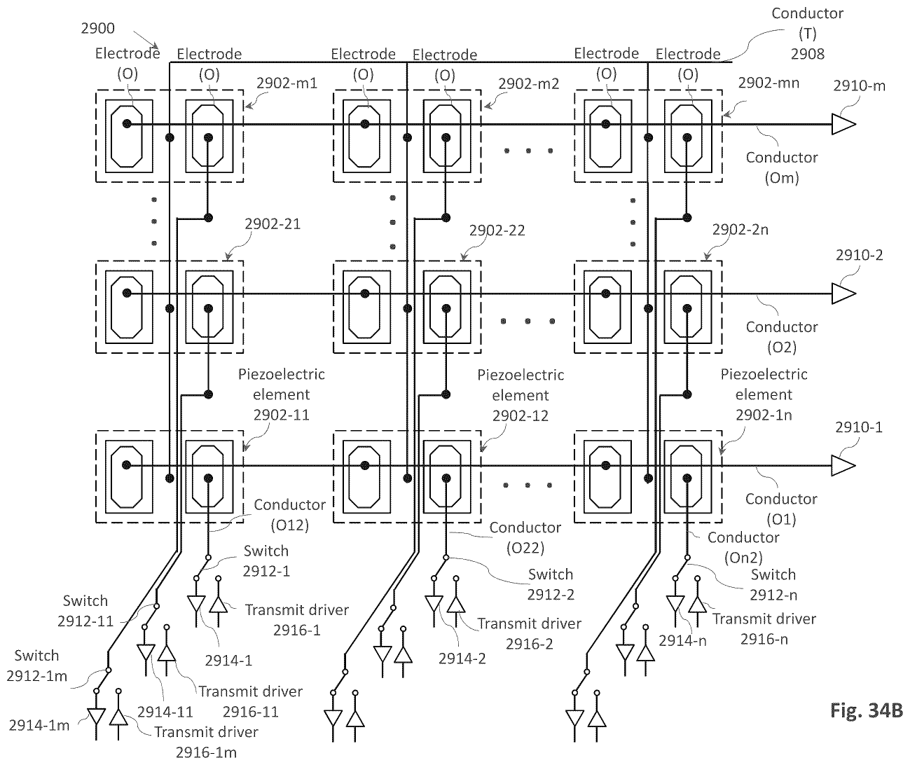

FIG. 34A shows an exemplary embodiment of an imaging system 2900 according to embodiments of the present disclosure. As depicted, the imaging system 2900 includes an array of piezoelectric elements 2902-11-2902-mn and each piezoelectric element may include first and second signal (O) electrodes and a T electrode. In embodiments, all of the T electrodes in the array may be electrically coupled to one common conductor (T) 2908; each row of the first O electrodes may be electrically connected to one of conductors O1-Om. If a line imager without synthetic lens is desired a mechanical lens may suffice. However, the same function can be achieved by not shorting all O nodes on a column as shown in FIG. 34A. Instead, each O node can be driven by a driver and if all driver signals for elements on a column have a same delay, same behavior as shown in FIG. 34A can be achieved. However, with the electronic way of implementing this as shown in FIG. 34B, different delays may be generated for elements on a column and achieve better focusing capability in the elevation plane and also have dynamic elevation focus that varies with depth as signal travels into target. In embodiments shown in FIG. 34A, each of the switches 2912-1-2912-n may toggle between a transmit driver (e.g., 2916-1) and an amplifier (e.g., 2914-1), which may be a low noise amplifier. In embodiments, each of the conductors O1-On may be connected to one of the amplifiers 2910-1-2910-m, which may be low noise amplifiers.

In embodiments, during the transmit mode, a signal may be transmitted from a transmit driver (e.g., 2916-1) to a column of second O electrodes via a conductor (e.g., O12) so that the column of piezoelectric elements may generate pressure waves as a line unit. During the transmit mode, each switch (e.g., 2912-1) may be toggled to a corresponding transmit driver (e.g., 2916-1).

In embodiments, the imaging system 2900 may process the reflected pressure waves in two different methods. In the first method, the amplifiers 2910-1-2910-n may receive electric charge signals from the first O electrodes, i.e., each amplifier may receive signals from a row of the first O electrodes. This method allows biplane imaging/mode, where for a two-dimensional image, the biplane image may provide orthogonal perspectives. Also, this method may provide more than two-dimensional imaging capabilities. The biplane imaging may be helpful for many applications, such as biopsy. It is noted that, in this method, the transmitting and receiving modes may be performed simultaneously. In the second method, the switches 2912 may be toggled to the amplifiers 2914 so that each amplifier may receive and process the electrical charge signals from a corresponding column of the second O electrodes.

Biplane imaging may be performed by first creating an image in the azimuth axis by applying delays to selected elements on columns. Elevation focus may also be achieved by adding additional delays to elements on the columns. In a subsequent operation, a second image is created on an orthogonal axis. This time, the image is developed on the elevation plane by applying delays to selected elements on rows. Additional delays may be added to elements on the rows to obtain slice thickness control in the azimuth direction. The two images are then synthetically added to display images in 2 orthogonal planes.

In embodiments, a line unit, which refers to a column (or row) of O electrodes electrically coupled to an O conductor, may operate as a transmit unit or a receive unit or both. In embodiments, even though the conductors O1-Om are arranged in orthogonal directions to the conductors O12-On2, the directions may be electronically programmed and electronically adjustable. For instance, the gain of the amplifiers 2910 and 2914 may be adjustable electronically, where gain control leads are implemented in the amplifiers. In embodiments, the length of each line elements (i.e., the number of piezoelectric elements in each line element) may also be electronically adjusted. In embodiments, this may be achieved by connecting all signal electrodes of every piezoelectric element to corresponding nodes in the ASIC chip and, where the ASIC programs the connection between the signal electrodes of the elements to be connected to each other, transmit drivers or amplifiers as appropriate.

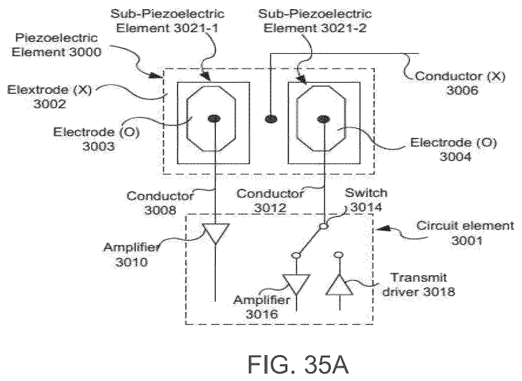

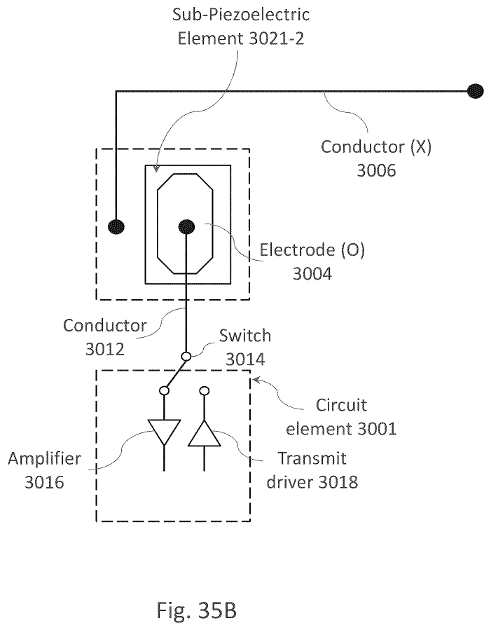

FIG. 35A shows an embodiment of a piezoelectric element 3000 coupled to a circuit element 3001 according to embodiments of the present disclosure. As depicted, the piezoelectric element 3000 may include: a first sub-piezoelectric element 3021-1 and a second sub-piezoelectric element 3021-2. The piezoelectric element 3000 may include: a bottom electrode (X) 3002 that is shared by the first and second sub piezoelectric elements and coupled to a conductor (X) 3006. In embodiments, the first sub-piezoelectric element 3021-1 may include a signal (O) electrode 3003 that is electrically coupled to the amplifier 3010 via the conductor 3008. In embodiments, the second sub-piezoelectric element 3021-2 may include a signal (O) electrode 3004 that is electrically coupled to the switch 3014 via the conductor 3012.

In embodiments, a circuit element 3001 may be electrically coupled to the piezoelectric element 3000 and include two amplifiers 3010 and 3016, such as low noise amplifiers, and a transmit driver 3018. In embodiments, the switch 3014 may have one end connected to the O electrode 3004 through the conductor 3012 and the other end that may toggle between the amplifier 3016 for the receive mode and a transmit driver 3018 for the transmit mode. In embodiments, the amplifier 3016 may be connected to other electronics to further amplify, filter and digitize a receive signal, even though an amplifier is used to symbolically represent the electronics. The transmit driver 3018 may be a multistage drive and may generate an output with two or more levels of a signaling. The signaling can be unipolar or bipolar. In embodiments, the transmit driver 3018 may include a switch interconnecting an input to an output of a driver under electronic control of the driver, which is not explicitly shown in FIG. 35A. Also not shown is the input signal to driver 3018, which may be delayed with respect to such signals for another element on the same column as shown in FIGS. 17A-17D. Similarly, delays with respect to elements located in different columns can also be implemented to allow electronic focus along azimuth axis, to allow for electronic focus along the elevation plane.

In embodiments, the signal of the transmit driver 3018 may be pulse width modulated (PWM), where, by controlling the pulse widths on a per element basis, a weighting function may be created on a transmitted ultrasound signal. This may for example perform a windowing function, where the transmit signal is weighted by a window function. In embodiments, the weighting coefficients may be achieved by varying the duty cycle of the transmit signal as is done during PWM signaling. This kind of operation may allow for transmit apodization, where the side lobes of a radiated signal are greatly attenuated, allowing for a higher quality image.

In embodiments, a transceiver array may be disposed in a transceiver substrate and include an n×n array of the piezoelectric element 3000 and an n×n array of the circuit elements 3001 may be disposed in an ASIC chip, where each piezoelectric element 3000 may be electrically coupled to a corresponding one of the n×n array of the circuit elements 3001. In such a case, the transceiver substrate may be interconnected to the ASIC chip by 3n² bumps. In embodiments, each column (or row) of the piezoelectric element array may be operated a line unit, as discussed in conjunction with FIGS. 33A and 33B as well as 34A and 33B. For instance, a same pulse may be simultaneously applied to a column of piezoelectric elements so that the column of piezoelectric elements may generate pressure waves simultaneously. It is noted that each piezoelectric element 3000 of the n×n array of piezoelectric elements may be coupled with a corresponding one circuit element 3001 of the n×n array of circuit elements. Alternately, each element on a column may be individually controlled by connecting the O node of an element to a dedicated Tx driver and also a dedicated receive amplifier. By controlling delays on the transmit driver and received signal from the LNA, elevation focus can be achieved in both the transmit and receive direction.

In embodiments, the sub-piezoelectric element 3021-1 may be in the receive mode during the entire operational period while the sub-piezoelectric element 3021-2 may be in either transmit or receive mode. In embodiments, the simultaneous operation of transmit and receive modes may allow the continuous mode Doppler imaging.

In embodiments, when the transmit driver 3018 transmits a signal to the electrode 3004, the power levels of the pressure waves generated by the sub-piezoelectric element 3021-2 may be changed by using pulse width modulation (PWM) signaling. This is important, for example, when switching from B mode to Doppler Mode imaging, signal power transmitted into the human body may be long and if power levels are not reduced, tissue damage may occur. Typically, in the conventional systems, different fast settling power supplies are used for B Mode and various Doppler Mode imaging to allow transmit drive voltages to differ in the 2 cases to for example not create excessive power in Doppler mode. Unlike the conventional systems, in embodiments, the power level may be changed by using the PWM signals on the transmit without using the conventional fast settling power supplies. In embodiments, rapid switching between Doppler and B mode imaging is desired to co-image these modes together. In embodiments, the ground electrodes of the piezoelectric element may also be separated from each other and connected to the ground separately. In embodiments, this independent grounding may reduce the noise and result in faster settling times. In embodiments, power transmitted may also be reduced by reducing the height of the transmit columns under electronic control. This again facilitates use of same power supply for both Doppler and B mode and meet power transmission requirements in each mode. This also allows co imaging.

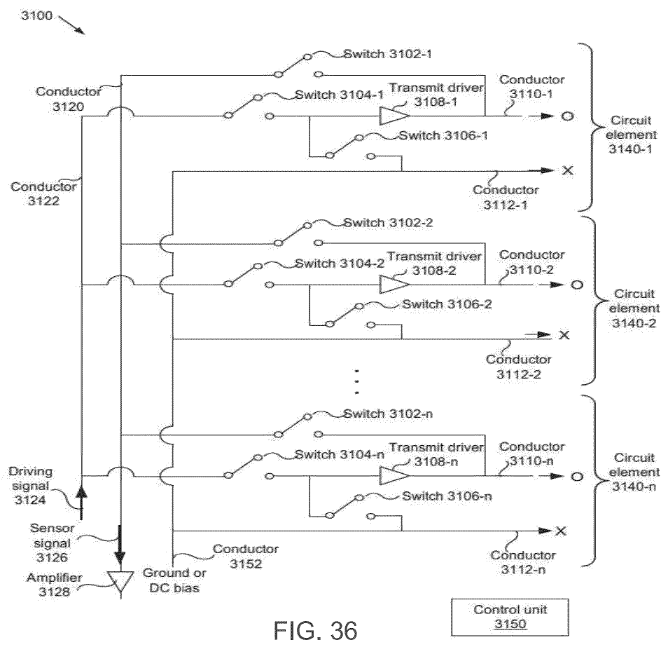

FIG. 36 shows a circuit 3100 for controlling multiple piezoelectric elements according to embodiments of the present disclosure. In embodiments, the circuit 3100 may be disposed in an ASIC chip, where the array (arranged in row and columns) of piezoelectric elements that is disposed in a transceiver substrate and the ASIC chip may be interconnected to the transceiver substrate by bumps, where each pMUT element may be connected to an associated Tx driver and receive circuitry through a switch as shown in FIG. 35B, with O electrode connecting to switch 3014. As depicted, the circuit 3100 may include an array of circuit elements 3140-1-3140-n, where each circuit element may communicate signals with the O and X electrodes of the corresponding piezoelectric element.

As depicted in FIG. 36, each circuit element (e.g., 3140-1) may include a first switch (e.g., 3102-1), a second switch (e.g., 3104-1), a third switch (e.g., 3106-1), and a transmit driver (e.g., 3108-1). The output from the transmit driver (e.g., 3108-1) may be sent to an O electrode of the piezoelectric element via a conductor (e.g., 3110-1). During the transmit mode, each circuit element may receive a transmit driver (driving) signal 3124 through a conductor 3122. Each second switch (e.g., 3104-1), which may be transistor switches and controlled by a control unit 3150, may be turned on to transmit the signal 3124 to the transmit driver (e.g., 3108-1). (The electrical connections between the control unit 3150 and other components in the circuit 3100 are not shown in FIG. 36.) The transmit driver (e.g., 3108-1) may perform logical decode, level shift, buffer the input signal and send the transit signal to the O electrode via the conductor (e.g., 3110-1). In embodiments, during the transmit mode, the first switch (e.g., 3102-1) may be turned off.

In embodiments, the control unit 3150 may decide which piezoelectric elements need to be turned on during the transmit mode. If the control unit 3150 decides not to turn on a second piezoelectric element, the first switch (e.g., 3102-2) and the second switch (e.g., 3104-2) may be turned off, while the third switch (e.g., 3106-2) may be turned on so that the O and X electrodes have the same electrical potential (i.e., there is a net zero volt drive across the piezoelectric layer). In in embodiments, the third switches 3106 may be optional.

In embodiments, during the receive mode, the first switch (e.g., 3102-1) may be turned on so that the electrical charge developed in the O electrode may be transmitted through the conductors 3110-1 and 3120 to the amplifier 3128. Then, the amplifier 3128 may receive electrical charge signal (or, equivalently, sensor signal) 3126 and amplify the sensor signal, where the amplified signal may be further processed to generate an image. During the receive mode, the second switch (e.g., 3104-1) and the third switch (e.g., 3106-1) may be turned off so that the received signal may not be interfered. It is noted that the entire array of the circuit element 3140-1-3140-n may share a common amplifier 3128, simplifying the design of the circuit 3100. In embodiments, the X electrodes of the piezoelectric elements may be electrically coupled to the ground or a DC bias voltage via the conductors 3112-1-3112-n, where the conductors 3112-1-3112-n may be electrically coupled to a common conductor 3152.

In embodiments, the circuit 3100 may be coupled to a column of piezoelectric elements (e.g., 2002-11-2002-n1) in FIG. 25. In embodiments, a plurality of circuits that are similar to the circuit 3100 may be coupled with the multiple columns of piezoelectric elements in the array in FIG. 30, and the conductors 3152 may be coupled to a common conductor (such as 2006 in FIG. 25). In embodiments, the circuit 3100 may control a column of piezoelectric elements in FIGS. 25-32.

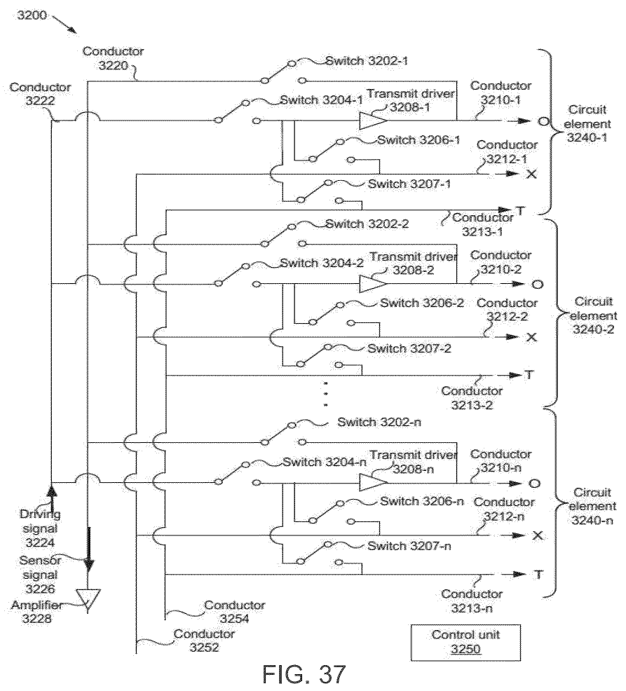

FIG. 37 shows a circuit 3200 for controlling multiple piezoelectric elements according to embodiments of the present disclosure. In embodiments, the circuit 3200 may be disposed in an ASIC chip, where the line (either column or row) of piezoelectric elements that is disposed in a transceiver substrate and the ASIC chip may be interconnected to the transceiver substrate by bumps. As depicted, the circuit 3200 may include an array of circuit elements 3240-1-3240-n, where each circuit element may communicate signals with the O, X, and T electrodes of the corresponding piezoelectric element.

As depicted in FIG. 37, each circuit element (e.g., 3240-1) may include a first switch (e.g., 3202-1), a second switch (e.g., 3204-1), a third switch (e.g., 3206-1), a fourth switch (e.g., 3207-1), and a transmit driver (e.g., 3208-1). The output from the transmit driver (e.g., 3208-1) may be sent to an O electrode of the piezoelectric element via a conductor (e.g., 3210-1). During the transmit mode, each circuit element may receive a transmit driver (or driving) signal 3224 through a conductor 3222. Each second switch (e.g., 3204-1), which may be a transistor switch and controlled by a control unit 3250, may be turned on to transmit the signal 3224 to the transmit driver (e.g., 3208-1). (The electrical connection between the control unit 3250 and other components in the circuit 3200 are not shown in FIG. 37.) The transmit driver (e.g., 3208-1) may logically decode the signal, level shift it and buffer, the output signal and send the transmit output signal to the O electrode via the conductor (e.g., 3210-1). In embodiments, during the transmit mode, the first switch (e.g., 3202-1) may be turned off.

In embodiments, the control unit 3250 may decide which piezoelectric elements need to be turned on during the transmit mode. If the control unit 3250 decides not to turn on a second piezoelectric element, the first switch (e.g., 3202-2) and the second switch (e.g., 3204-2) may be turned off, while the third switch (e.g., 3206-2) and the fourth switch (e.g., 3207-2) may be turned on so that the O and X (and T) electrodes have the same electrical potential (i.e., there is a net zero volt drive across the piezoelectric layer). In in embodiments, the third and fourth switches (e.g., 3206-2 and 3207-2 may be optional. It is understood that 3 level signaling and a transmit driver that performs that is not shown explicitly. Similarly, the connections to X T conductors and switches like 3206-2, 3207-2 are shown in a simplified manner.

In embodiments, during the receive mode, the first switch (e.g., 3202-1) may be turned on so that the electrical charge developed in the O electrode may be transmitted through the conductors 3210-1 and 3220 to the amplifier 3228. Then, the amplifier 3228 may amplify the electrical charge (or sensor) signal 3226, where the amplified signal may be further processed to generate an image. During the receive mode, the second switch (e.g., 3204-1), the third switch (e.g., 3206-1), and the fourth switch (e.g., 3207-1) may be turned off so that the received signal may not be interfered.

It is noted that the entire array of the circuit element 3240-1-3240-n may share a common amplifier 3228, simplifying the design of the circuit 3200. In embodiments, the X electrodes of the piezoelectric elements may be electrically coupled to the ground or a DC bias voltage via the conductors 3212-1-3212-n, where the conductors 3212-1-3212-n may be electrically coupled to a common conductor 3252. In embodiments, the T electrodes of the piezoelectric elements may be electrically coupled to the ground or a DC bias voltage via the conductors 3213-1-3213-n, where the conductors 3213-1-3213-n may be electrically coupled to a common conductor 3254.

In embodiments, the circuit 3200 may be coupled to a column of piezoelectric elements (e.g., 2102-11-2102-n1) in FIG. 26. In embodiments, a plurality of circuits that are similar to the circuit 3200 may be coupled with the multiple columns of piezoelectric elements in the array in FIG. 26, and the conductors 3252 may be coupled to a common conductor (such as 2106 in FIG. 26). Similarly, in embodiments, the conductors 3254 may be coupled to a common conductor (such as 2108 in FIG. 26). In embodiments, the circuit 3200 may control a column of piezoelectric elements in FIGS. 25-32.

In FIGS. 27-37, conductors are used to electrically couple an electrode to another electrode. For instance, the electrodes 2006-11-2006-m1 are electrically coupled to a conductor 2006. In embodiments, the conductors in FIGS. 27-37 may be implemented in a variety of methods, such as metal interconnect layers deposited and patterned on the substrate on which the piezoelectric elements are disposed or on a different substrate, such as ASIC, that is connected to the substrate.

FIGS. 38 and 39 show exemplary waveforms 3300 and 3400 for driving a piezoelectric element during the transmit mode according to embodiments of the present disclosure. In general, piezoelectric material may be vulnerable to damages caused by dielectric aging, and the aging may be delayed or avoided by using unipolar drive signals. The waveforms 3300 and 3400 represent the voltage potential between O and X electrodes and/or between O and T electrodes. As depicted, the waveforms may be unipolar in nature and may be a two-level step waveform 3300 (i.e., the transmit driver, such as 2812, 2912, 3018, 3108, 3208, etc. is a unipolar transmit driver) or a multilevel (such as three level) step waveform 3400. The actual voltage amplitude may vary typically from 1.8 V to 12.6 V. In embodiments, the multistep waveform 3400 or a waveform with more steps may reduce heating in the piezoelectric element and have advantages for use during certain imaging modes, such as Doppler or harmonic imaging.

In embodiments, the frequency of the pulses in the waveforms 3300 and 3400 may vary depending on the nature of the signal needed and need to contain the frequency at which membrane underlying the pMUT is responsive to. In embodiments, the waveforms may also be complex signals, such as linear or non-linear frequency modulated chirp signals, or other coded signals using the Golay codes.

In embodiments, the circuits for driving the piezoelectric elements may further be designed such that the transmit output from the underlying membrane may be symmetrical in shape. In embodiments, for each signal pulse in the waveform 3300 (or 3400), the rising edge of the pulse may be substantially symmetrical to the falling edge of the pulse with respect to the center of the pulse. This symmetry lowers the harmonic content of the transmit signal, especially the second harmonic and other even order harmonics signal. In embodiments, the signal pulse in the waveform 3300 (or 3400) may be a pulse width modulated (PWM) signal.

FIG. 40 shows a transmit drive signal waveform according to embodiments of the present disclosure. As depicted, the signal 3500 from the transmit driver may be symmetric and bipolar, i.e., the magnitude (H1) and width (W1) of the peak maximum voltage are the same as the magnitude (H2) and width (W2) of the peak minimum voltage. Also, the slope of the rising edge 3502 is the same as the slope of the falling edge 3504. In addition, the rising time W3 is the same as the fall time W4, where the fall time W4 refers to the time interval between the starting point of the fall and the reference voltage. Furthermore, the rising edge 3506 has the same slope as the rising edge 3502.

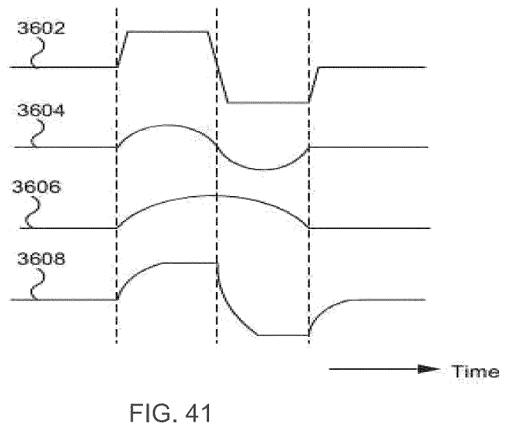

During the transmit operation, the transmit drive, e.g., 3018 in FIG. 35, may be driven by an electrical waveform, such as shown in FIGS. 38 and 39. FIG. 41 shows output signals of various circuits in an imaging assembly according to embodiments of the present disclosure. In embodiments, the waveform 3602 may be an output signal from the transmit driver, e.g., 3018 and transmitted to a piezoelectric element, e.g., 3000. In embodiments, as the piezoelectric element may have an inherent bandwidth, it may output a sinusoidal output 3604 at its resonant frequency. If the output of the transmit driver connected to the O electrode of the piezoelectric element rises very slowly, it may not be able to charge the electrode to the desired final value and thus may cause low output signals, as shown in waveform 3606, where final amplitude is smaller than in 3602. On the other hand, if the output signal of the transmit driver settles very quickly, the output signal of the transmit driver has larger bandwidth than the bandwidth limit of the piezoelectric element and therefore extra energy may be dissipated in heat. Therefore, in embodiments, as shown in the waveform 3608, the piezoelectric element may be charged at a rate such that it is completely charged but not very quickly. In embodiments, the waveform 3608, which represents the voltage potential across the top and bottom electrodes as a function of time, is closer in shape to the output of the transducer and because difference in shape is smaller, the input signal bandwidth and output signal bandwidth matches better, less loss of energy in heat occurs. In embodiments, drive impedance of transmit driver is optimized to reduce the loss of energy. Stated differently, the impedance of the transmit driver is designed to drive the piezoelectric element optimally with respect to heat dissipation and time constants needed for adequate voltage settling within a target time period.

In embodiments, the imager 126 may use a harmonic imaging technique, where the harmonic imaging refers to transmitting pressure waves on the fundamental frequency of the membrane and receiving reflected pressure waves at second or higher harmonic frequencies of the membrane. In general, the images based on the reflected waves at the second or higher harmonic frequencies have higher quality than the images based on the reflected waves at the fundamental frequency. The symmetry in the transmit waveform may suppress the second or higher harmonic components of the transmit waves, and as such, the interference of these components with the second or higher harmonic waves in the reflected waves may be reduced, enhancing the image quality of the harmonic imaging technique. In embodiments, to reduce the second or higher harmonic waves in the transmit waves, the waveform 3300 may have 50% duty cycle.

In FIGS. 25-34, the arrays may include multiple line units, where each line unit includes a plurality of piezoelectric elements that are electrically coupled to each other. In embodiments, the line units may be driven with multiple pulses that have phase differences (or equivalently delays). By adjusting the phases, the resultant pressure waves may be steered at an angle, which is referred to as beamforming.

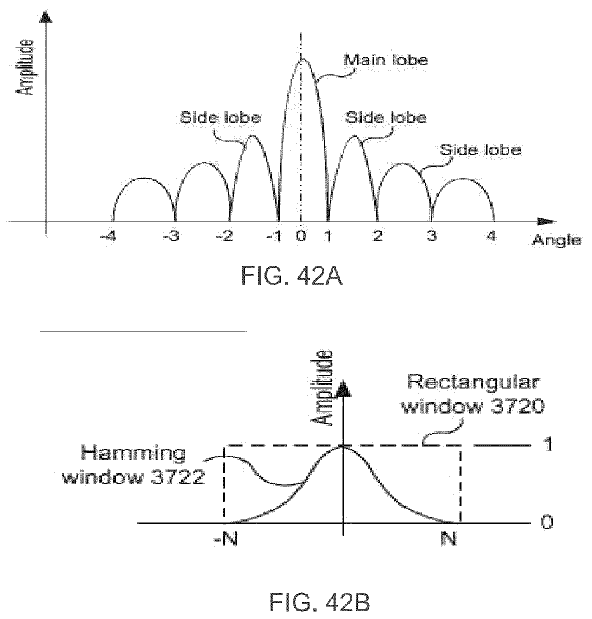

FIG. 42A shows a plot of the amplitude of a transmit pressure wave as a function of spatial location along the azimuth axis of the transducer according to embodiments of the present disclosure. If the piezoelectric elements in the array are arranged in 2 dimensions and the piezoelectric elements on a column in the Y direction are connected and have many columns along the X direction, the X direction is known as the azimuth direction and the Y direction is known as the elevation direction. As depicted in FIG. 37A, the transmit pressure wave includes the main lobe and multiple side lobes. The main lobe may be used to scan tissue targets and have high pressure amplitude. The side lobes have lower amplitude but degrade quality of images and therefore it is desirable to reduce their amplitude.

In some embodiments, apodization herein includes using variable voltage drive, for example, with lower weights near edges and fuller weights near the central parts of ultrasonic pulses. Apodization may also be implemented by changing the number of elements along each column or rows, either alone or in combination with other methods disclosed herein.

FIG. 42B shows various types of windows for apodization process according to embodiments of the present disclosure. In FIG. 42B, x-axis represents position of a piezoelectric element relative to the piezoelectric element at the center of an active window and y-axis represents the amplitude (or, weight applied to the piezoelectric element). As depicted, for the rectangular window 3720, there is no weighting provided for any of the transmit lines, i.e., they are all at a uniform amplitude (i.e., symbolically 1). On the other hand, if the weighting function is implemented, as depicted by the Hamming window 3722, lines at the center get a greater weighting than ones at the edges. For instance, to apply the Hamming window 3722 to the transducer tile, the piezoelectric elements in the leftmost column (which is denoted as—N in FIG. 42B) and the piezoelectric elements in the rightmost column (which is denoted as N in FIG. 42B) may have the lowest weight, while the piezoelectric elements in the middle column may have the highest weight. This process is known as apodization. In embodiments, various types of window weighting may be applied, even though the Hamming window 3722 shown is only meant to be one example. In embodiments, apodization may be implemented by a variety of means such as scaling the transmit driver output drive level differently for different lines by employing a digital to analog converter (DAC) or by keeping the same drive level but reducing the number of pixels on a line The net effect is the side lobe level can be reduced by use of apodization, where the weighting of the transmit drive varies based on where a particular line is located within the transmit aperture energized.

In embodiments, the reduction in the voltage of the pulses or waveforms may lower the temperature at the transducer surface. Alternately, for a given maximum acceptable transducer surface temperature, transducers operating at lower voltages may deliver better probe performance, resulting in better quality images. For example, for a probe with 192 piezoelectric elements to reduce power consumption, transmit pressure waves may be generated by using only a portion of probe (i.e., a subset of the piezoelectric elements) and scanning the remaining elements sequentially in time using a multiplexer. Therefore, at any point of time, in the conventional systems, only a portion of the transducer elements may be used to limit the temperature rise. In contrast, in embodiments, the lower voltage probe may allow more piezoelectric elements to be addressed simultaneously, which may enable increased frame rates of the images and enhanced image quality. Significant power is also consumed in the receive path where the received signal is amplified using LNAs. An imaging system typically uses a number of receive channels, with an amplifier per receiver channel. In embodiments, using temperature data, a number of receiver channels can be turned off to save power and reduce temperature.

In embodiments, the apodization may be achieved by varying the number of piezoelectric elements in each line unit according to a window function. In embodiments, such a window approximation may be achieved by electronically controlling the number of piezoelectric elements on a line or by hardwiring the transducer array with the required number of elements. Apodization can also be created by using a fixed number of elements, but driving these elements with varying transmit drive voltage. For example, for apodization in the elevation direction, maximum drive is applied to central elements on the column and lower driver levels are applied to outer elements on both side of the column around the central element on the column. Apodization can also be achieved by varying the poling strength of elements based on location on a column.

In general, the heat developed by a probe may be a function of the pulse duration in the transmit pulse/waveform. In general, to make the pressure waves penetrate deep in the target with better signal to noise ratio (SNR), a piezoelectric element may require long pulse trains. However, this also degrades axial resolution and also generates more heat in the piezoelectric elements. So, in the conventional systems, the number of pulses emitted is small, sometimes one or two. Since longer pulses may create more heat energy, making it impractical for their use in the conventional systems. In contrast, in embodiments, the pulses and waveforms 3300 and 3400 may have significantly lower peak values, which may enable the use of long pulse trains, chirps or other coded signaling. In embodiments, the longer pulse trains do not degrade axial resolution since in the receiver matched filtering is performed to compress the waveform to restore resolution. This technique allows a better signal to noise ratio and allows signals to penetrate deeper into the body and allows for high quality imaging of targets deeper in the body.

In embodiments, a layer of Polydimethylsiloxane (PDMS) or other impedance matching material may be spun over the transducer elements. This layer may improve the impedance matching between the transducer elements and the human body so that the reflection or loss of pressure waves at the interface between the transducer elements and the human body may be reduced.

In FIGS. 25-34, more than one line unit may be created by connecting pixels in the y-direction (or x-direction), where one line unit (or equivalently line element) refers to multiple piezoelectric elements that are electrically connected to each other. In embodiments, one or more line units may also be created by connecting piezoelectric elements along the x-direction. In embodiments, the piezoelectric elements in a line unit may be hardwired.

As discussed in conjunction with FIG. 24A, each piezoelectric element 1806 may be electrically coupled to a circuit 1842, i.e., the number of piezoelectric elements in the transceiver substrate 1802 is the same as the number of circuit 1842 in the ASIC chip 1804. In such a case, the electrical connections of piezoelectric elements in each column (or row) may be performed electronically, i.e., the hardwire conductors (e.g., 2006) for connecting electrodes in a column (or row) is replaced by electronic switches. Stated differently, the piezoelectric elements in a line imager/unit may be electronically connected to each other. For an electronically controlled line imager, a line imager/unit may be built by connecting each piezoelectric element of a two dimensional matrix array to a corresponding control circuit (such as 1842) of a two dimensional array of control circuits, where the control circuits are located spatially close to pixels. To create a line element, a multiplicity of drivers controlling a column (or row) of pixels may be turned on electronically. In embodiments, the number of drivers in each line imager/unit can be electrically modified under program control and electronically adjustable.

In embodiments, smaller capacitance of each pixel may be driven efficiently by the distributed drive circuitry without other equalizing elements in between driver and pixel, eliminating the difficulty of driving a very large line capacitance. In embodiments, driver optimization may allow symmetry in rising edge and falling edges, allowing better linearity in transmit output, enabling harmonic imaging. (The symmetry is described in conjunction with FIGS. 38 and 39.) In embodiments, electronic control may allow programmable aperture size, transmit apodization, and horizontal or vertical steering control, all of which may improve image quality. In embodiments, the configurable line imager/unit under electronic control may be electrically modified under program control. For example, if a smaller number of connected elements is desired in the y-direction, the number may be adjusted by software control and without having to re-spin the control electronic circuitry or the piezoelectric array.

In embodiments, each line unit may be designed to consist of several subunits with separate control for each sub unit. The advantage of these subunits is that they may alleviate the difficulty of driving a large capacitive load for a line unit using one single external transmit driver. For example, if two line units are created in the place of one line unit that includes the entire piezoelectric elements in a column, two different transmit drivers (such as 2816) may be employed and each transmit driver may control half of the load of the full line unit. Also, even if one driver is used, driving the first half of the line unit and the second half of the line unit separately may improve the drive situation due to lower resistance connection to both ends of the line unit.

In embodiments, both the length and orientation of the line units may be controlled. For instance, in FIGS. 25-34, the line units may be arranged in both x and y directions. By way of example, in FIG. 35, the O electrodes along a column (e.g., 2003-11-2003-n1) may be electrically coupled to form one line unit, and the O electrodes in the other columns may be electrically coupled to form n number of line units that extend along the x-direction. More specifically, the line units that extend along the x-direction include n number of O electrodes (2003-12-2003-1n), . . . , (2003-n2-2003-nn). In embodiments, the arrangement of line units along orthogonal directions may be possible by controlling the electrical circuits in ASIC chip.

In FIGS. 25-35, each piezoelectric element may include two or more top (X and T) electrodes. In embodiments, the piezoelectric layer under these top electrode may be poled in the same direction or opposite directions. The multiple poling direction when combined with an appropriate applied signal electric field may create improvements in transducer transmit and receive sensitivities and also create additional resonances to enable wider bandwidth.

In FIGS. 25-35, each array may have one or more membranes disposed under the piezoelectric elements. In embodiments, the membranes may have multiple modes of vibration.

In embodiments, one membrane may vibrate in the fundamental mode at a certain frequency while another membrane may vibrate at a different frequency determined by membrane design and relative arrangements of electrodes with different poling directions. In embodiments, multiple membranes may be driven by same electrode set and each membrane may have different fundamental frequencies. In embodiments, each membrane may be responsive to a wide range of frequencies, increasing its bandwidth. Also, such a transducer with different poling directions may help increase transmit and receive sensitivities while also enabling a high bandwidth transducer.

In some embodiments, the X (or T) electrodes in a column may be electrically coupled to a conductor. In embodiments, these conductors may be electrically coupled to one common conductor. For instance, the conductors may be electrically coupled to one common conductor line so that all of the T electrodes in the array may be connected to the ground or a common DC bias voltage.

In some embodiments, each array may include piezoelectric elements that are arranged in a two-dimensional array (e.g., FIGS. 25-34), where the number of elements in the x-direction may be the same as the number of elements in the y-direction. However, it should be apparent to those of ordinary skill in the art that the number of elements in the x-direction may be different from the number of elements in the y-direction.

In embodiments, the ASIC chip (such as 1804) coupled to the transducer substrate (such as 1802) may contain temperature sensors that measure the surface temperatures of the imaging device 120 facing the human body during operation. In embodiments, the maximum allowable temperature may be regulated, and this regulation may limit the functionality of the imaging device since the temperatures should not rise beyond the allowable upper limit. In embodiments, this temperature information may be used to improve image quality. For example, if temperature is below the maximum allowed limit, additional power may be consumed in the amplifiers to lower its noise and improve system signal-to-noise ratio (SNR) for improved quality images.

In embodiments, the power consumed by the imaging device 126 increases as the number of line units that are driven simultaneously increases. All line units in the imaging device 126 may need to be driven to complete transmitting pressure waves from the whole aperture. If only a few line units are driven to transmit pressure waves, wait and receive the reflected echo at a time, it will take more time to complete one cycle of driving the entire line units for the whole aperture, reducing the rate at which images can be taken per second (frame rate). In order to improve this rate, more line units need to be driven at a time. In embodiments, the information of the temperature may allow the imaging device 120 to drive more lines to improve the frame rate.

In some embodiments, each piezoelectric element may have one bottom electrode (O) and one or more top electrodes (X and T) and have more than one resonance frequency. For instance, each piezoelectric element 2502 in FIG. 30 may have one bottom electrode (O) and two top electrodes, where the first top electrode and the bottom electrode (O) may be responsive to a first frequency f1, while the second top electrode and the bottom electrode (O) may be responsive to a second frequency f2 that may be different from f1.

In embodiments, the electrical charge developed during the receive mode is transferred to an amplifier, such as 1811, 2810, 2814, 2910, 2914, 3010, 3016, 3128, and 3228. Then, the amplified signal may be further processed by various electrical components. As such, it should be apparent to those of ordinary skill in the art that the each of the amplifiers 1811, 2810, 2814, 2910, 2914, 3010, 3016, 3128, and 3228 collectively refers to one or more electrical components/circuits that process the electrical charge signal, i.e., each amplifier symbolically represents one or more electrical components/circuits for processing the electrical charge signal.

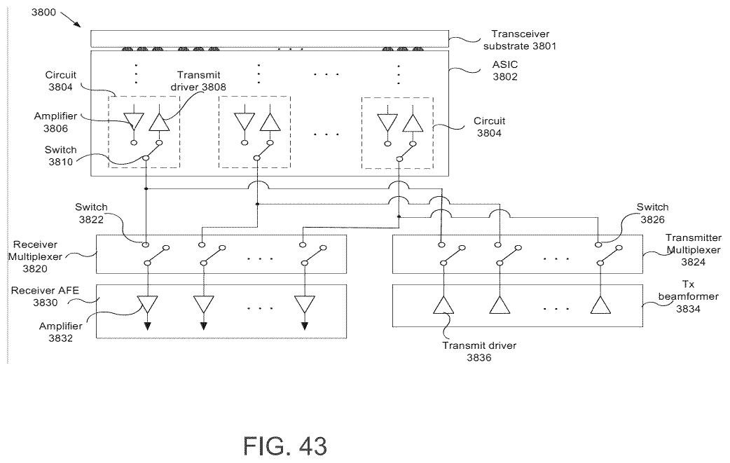

FIG. 43 shows a schematic diagram of an imaging assembly 3800 according to embodiments of the present disclosure. As depicted, the imaging assembly 3800 may include: a transceiver substrate 3801 having piezoelectric elements (not shown in FIG. 38); an ASIC chip 3802 electrically coupled to the transceiver substrate 3801; a receiver multiplexer 3820 electrically coupled to the ASIC chip 3802; a receiver analogue-front-end (AFE) 3830; a transmitter multiplexer 3824 electrically coupled to the ASIC chip 3802; and a transmit beamformer 3834 electrically coupled to the second multiplexer 3824. In embodiments, the ASIC chip 3802 may include multiple circuits 3804 that are connected to and configured to drive multiple piezoelectric elements in the transceiver substrate 3801. In embodiments, each circuit 3804 may include a receiver amplifier (or shortly amplifier) 3806, such as LNA, and a transmit driver 3808 for transmitting a signal to a piezoelectric element, and a switch 3810 that toggles between the amplifier 3806 and the transmit driver 3808. The amplifiers may have programmable gain and means to connect them to piezo elements that need to be sensed. The transmit drivers have means to optimize their impedance and means to be connected to piezoelectric elements that are to be driven.

In embodiments, the receiver multiplexer 3820 may include multiple switches 3822 and the receiver AFE 3830 may include multiple amplifiers 3832. In embodiments, each of the switches 3822 may electrically connect/disconnect a circuit 3804 to/from an amplifier 3832. In embodiments, the transmitter multiplexer 3824 may include multiple switches 3826 and the transmit beamformer 3834 may include multiple transmit driver 3836 and other circuitry not shown to control the relative delay between transmit driver waveform of the various drivers, and other circuitry not shown to control the frequency and the number of pulses for each of the transmit drivers. In embodiments, each of the switches 3826 turn on during a transmit operation and connect to circuit 3804, while switches 3822 turn off, while switch 3810 connects to transmit driver 3808. Similarly, during a receive operation, switches 3826 turn off while switches 3822 turn on, while switch 3810 is connected to amplifier 3806.

In embodiments, the switches 3810 may be toggled to the transmit drivers 3808 during the transmit mode and toggle to the amplifiers 3806 during the receive mode. In embodiments, a portion of the switches 3822 may be closed so that the corresponding circuits 3804 may be set to the receive mode. Similarly, a portion of the switches 3826 may be closed so that the corresponding circuits 3804 may be set to the transmit mode. Since a portion of the switches 3822 and a portion of the switches 3826 may be closed simultaneously, the imager assembly may be operated in both transmit and receive modes simultaneously. Also, the receiver multiplexer 3820 and the transmitter multiplexer 3824 reduce the number of ASIC pins. In embodiments, the receiver multiplexer 3820, receiver AFE 3830, transmitter multiplexer 3824, and transmitter beamformer 3834 may be included in the circuits 202a or portions may also reside in 215a in FIG. 1B.

In embodiments, each piezoelectric element may have more than two electrodes, where one electrode may be in the transmit mode to generate pressure waves while the other electrode may be simultaneously in the receive mode to develop electrical charge. This simultaneous operation of transmit and receive modes allow for better Doppler imaging.

Movement in target being imaged may cause errors in the resulting image and it may be desirable to reduce these errors. An example of movement is when performing cardiac imaging where the heart tissue is moving. High frame rates can be desirable to reduce impact of movements. Therefore, improving frame rates while maintaining electronic azimuth and elevation focus and apodization can be important. This may not only reduce burring in images but also allow for better images using dynamic focus in the receiver by electronically altering azimuth and electronic focus as a function of depth. Frame rate improvement can be achieved in a dual stage beamformer illustrated in FIG. 16, by simultaneously operating the top and bottom section at same time, reducing the number of operation. Further, by completing the scan of one complete column, for example A1, B1 and C1 of FIG. 14 before generating A2,B2,C2 helps minimize impact of movements on a line. Further, one scan line can be created by using the transmission and reception of all rows and columns in the section operated. However, using a parallel beam former technique [High frame rate ultrasound imaging using parallel beamforming, Tore Grüner Bjåstad, Thesis for the degree of Philosophiae Doctor Trondheim, January 2009 Norwegian University of Science and Technology], multiple beams can be created, for example, four. This can help further increase the frame rate and reduces impact of movements. These techniques also may create aberrations, but there are known electronic ways to correct them.

In some embodiments, although electronic or electrical connections between individual elements shown in figures herein are hardwired or physical connections, different digital connections may be used to thus enable programmable and more flexible digital communications. In some embodiments, such digital connections may include but not limited to switches, plugs, gates, connectors, etc.

In some embodiments, 3D imaging may be performed using a 2D array of transducer elements as disclosed herein. The azimuth plane may be addressed by controlling delays of column elements. This delay control may be similar to that used in B mode imaging. 3D imaging may create volumes in 3D space and therefore the elevation plane may need to be addressed. In an exemplary embodiment, ultrasound beams can be steered in the elevation plane for a transmission from the entire transducer array. In this case, the beam is focused in the azimuth plane by controlling delays in the azimuth direction. Elevation control may be achieved by controlling delay for elements on a column consistent with steering a beam on the elevation plane, for example, all column elements for all columns. In this exemplary embodiment, one scan line in the azimuth plane is obtained by transmitting from multiple columns, e.g., 128 columns, with bottom element of each column element varying with respect to another similar column as needed to focus the beam in the azimuth plane. In the same embodiment, the element on the column may have constant delay increase starting from the element on row 0, consistent to steering a beam in the elevation plane. These steps can then be repeated for multiple times, for example 100 times, picking a different region to focus the beam in the azimuth plane, but maintaining the same elevation delays to maintain same beam steering in the elevation direction. This can then generate 100 scan lines at an elevation angle. This may then be followed by another 100 transmit events with similar azimuth focus as previously, but elevation steering is done using different delays for elements on a column, resulting in a different steering angle. Many different steering angles may be performed to scan a volume. Different steering angles are shown in FIG. 44. The resulting echo signal may be received in the transducer and image may be reconstructed. To speed up frames per second, parallel beam forming may be performed and phase aberrations may be corrected for a high-quality image. Although certain embodiments and examples are provided in the foregoing description, the inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

As used herein A and/or B encompasses one or more of A or B, and combinations thereof such as A and B. It will be understood that although the terms “first,” “second,” “third” etc. may be used herein to describe various elements, components, regions and/or sections, these elements, components, regions and/or sections should not be limited by these terms. These terms are merely used to distinguish one element, component, region or section from another element, component, region or section. Thus, a first element, component, region or section discussed below could be termed a second element, component, region or section without departing from the teachings of the present disclosure.

The terminology used herein is for the purpose of describing particular embodiments only and is not intended to limit the present disclosure. As used herein, the singular forms “a,” “an,” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

As used in this specification and the claims, unless otherwise stated, the term “about,” and “approximately,” or “substantially” refers to variations of less than or equal to +/−0.1%, +/−1%, +/−2%, +/−3%, +/−4%, +/−5%, +/−6%, +/−7%, +/−8%, +/−9%, +/−10%, +/−11%, +/−12%, +/−14%, +/−15%, or +/−20% of the numerical value depending on the embodiment. As a non-limiting example, about 100 meters represents a range of 95 meters to 105 meters (which is +/−5% of 100 meters), 90 meters to 110 meters (which is +/−10% of 100 meters), or 85 meters to 115 meters (which is +/−15% of 100 meters) depending on the embodiments.

While preferred embodiments have been shown and described herein, it will be obvious to those skilled in the art that such embodiments are provided by way of example only. Numerous variations, changes, and substitutions will now occur to those skilled in the art without departing from the scope of the disclosure. It should be understood that various alternatives to the embodiments described herein may be employed in practice. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the disclosure and that methods and structures within the scope of these claims and their equivalents be covered thereby.

What is claimed is:

1. An ultrasonic imaging system comprising:
a) an ultrasonic transducer comprising a plurality of pMUT transducer elements, each of the plurality of pMUT transducer elements having two or more terminals; and
b) one or more circuitries connected to the plurality of pMUT transducer elements, the one or more circuitries electronically configured to enable:
i) ultrasonic pulse transmission from the ultrasonic transducer;
ii) receiving a reflected ultrasonic signal at the ultrasonic transducer; and
iii) electronic control configured to focus the ultrasonic pulse or the reflected ultrasonic signal in an elevation direction.
